(19) United States
(12) Patent Application Publication
(10) Pub. No.: US 2026/0047925 A1
(43) Pub. Date: Feb. 19, 2026

(54) EXPANDABLE FRAME FOR MEDICAL DEVICE

(71) Applicant: MiRus LLC, Marietta, GA (US)

(72) Inventors: Jay Yadav, Atlanta, GA (US); Alex Panter, Marietta, GA (US); Noah Roth, Marietta, GA (US); Jordan Bauman, Smyrna, GA (US); Ravi Enneti, Marietta, GA (US)

(21) Appl. No.: 19/367,168

(22) Filed: Oct. 23, 2025

Related U.S. Application Data
(63) Continuation-in-part of application No. 18/429,919, filed on Feb. 1, 2024.
Application No. 18/429,919 is a continuation-in-part of application No. 18/417,939, filed on Jan. 19, 2024.
Application No. 18/429,919 is a continuation-in-part of application No. 18/418,007, filed on Jan. 19, 2024.
Application No. 18/429,919 is a continuation-in-part of application No. 18/400,338, filed on Dec. 29, 2023.
Application No. 18/429,919 is a continuation-in-part of application No. 18/204,180, filed on May 31, 2023.
(60) Provisional application No. 63/347,337, filed on May 31, 2022.
(60) Provisional application No. 63/389,281, filed on Jul. 14, 2022.
(60) Provisional application No. 63/439,892, filed on Jan. 19, 2023.
(60) Provisional application No. 63/439,908, filed on Jan. 19, 2023.
(60) Provisional application No. 63/540,266, filed on Sep. 25, 2023.
(60) Provisional application No. 63/540,556, filed on Sep. 26, 2023.

(51) Int. Cl. A61F 2/24; A61L 27/04; A61L 27/06; A61L 27/30; A61L 27/36

(52) U.S. Cl. CPC A61F 2/2418; A61L 27/047; A61L 27/06; A61L 27/306; A61L 27/3604

(57) ABSTRACT

A prosthetic heart valve for the treatment of structural heart disease wherein the prosthetic heart valve includes an expandable frame that a) has an open cell geometry in the frame of the prosthetic heart valve that can be used to reduce delivery system size, b) has high radial strength, c) has improved restoration of the physiologic EOA, d) has lower recoil, e) has little or no longitudinal foreshortening, f) allows for proper placement of the bioprosthetic valve in relation to the native commissures of the valve, h) has symmetrical and cylindrical expansion of the prosthetic valve resulting in lower rates of leaflet thrombosis and structural valve deterioration, and/or i) prevents allergic response and restenosis associated with nickel content.

### REFERENCED APPLICATIONS

The present application is a continuation-in-part of U.S. application Ser. No. 18/429,919 filed Feb. 1, 2024, which in turn claims priority to U.S. Provisional Application Ser. No. 63/540,556 filed Sep. 26, 2023, which are incorporated herein by reference.

The present disclosure is a continuation-in-part of U.S. application Ser. No. 18/429,919 filed Feb. 1, 2024, which in turn is a continuation-in-part of U.S. application Ser. No. 18/417,939 filed Jan. 19, 2024, which in turn claims priority on United States Provisional Application Ser. No. Ser. No. 63/439,892, filed Jan. 19, 2023, which are all fully incorporated herein by reference.

The present disclosure is a continuation-in-part of U.S. application Ser. No. 18/429,919 filed Feb. 1, 2024, which in turn is a continuation-in-part of U.S. application Ser. No. 18/418,007 filed Jan. 19, 2024, which in turn claims priority on United States Provisional Application Ser. No. Ser. No. 63/439,908, filed Jan. 19, 2023, which are all fully incorporated herein by reference.

The present application is a continuation-in-part of U.S. application Ser. No. 18/429,919 filed Feb. 1, 2024, which in turn is a continuation-in-part of U.S. application Ser. No. 18/400,338 filed Dec. 29, 2023, which in turn priority claims priority to U.S. Provisional Application Ser. No. 63/540,266 filed Sep. 25, 2023, which are all fully incorporated herein by reference.

The present application is a continuation-in-part of U.S. application Ser. No. 18/429,919 filed Feb. 1, 2024, which in turn is a continuation-in-part of U.S. application Ser. No. 18/204,180 filed May 31, 2023, which claims priority on U.S. Provisional Application Ser. No. 63/389,281 filed Jul. 14, 2022, which are incorporated herein by reference.

The present application is a continuation-in-part of U.S. application Ser. No. 18/429,919 filed Feb. 1, 2024, which in turn is a continuation-in-part of U.S. application Ser. No. 18/204,180 filed May 31, 2023, which claims priority on U.S. Provisional Application Ser. No. 63/347,337 filed May 31, 2022, which are incorporated herein by reference.

### FIELD OF DISCLOSURE

The disclosure relates generally to medical devices and medical device applications, and particularly to a medical device that includes an expandable frame, more particularly to a medical device in the form of a cardiovascular implant for the treatment of structural heart disease wherein the cardiovascular implant includes an expandable frame, and still more particularly to a medical device in the form of a prosthetic heart valve for the treatment of structural heart disease wherein the prosthetic heart valve includes an expandable frame.

### BACKGROUND OF DISCLOSURE

Many cardiovascular devices such as expandable heart valves, and the like are inserted into a patient via the vascular system of a patient and then expanded at the treatment site. These devices are typically crimped onto a catheter prior to insertion into a patient.

Medical devices such as Transcatheter aortic replacement valves (TAVR valves) represent a significant advancement in prosthetic heart valve technology. TAVR valves bring the benefit of heart valve replacement to patients that would otherwise not be operated on. Transcatheter aortic valve replacement (TAVR) can be used to treat aortic valve stenosis in patients who are classified as high-risk for open heart surgical aortic valve replacement (SAVR). Non-limiting TAVR valves are disclosed in US Patent Nos. U.S. Pat. Nos. 5,411,522; 6,730,118; 10,729,543; 10,820,993; 10,856,970; 10,869,761; 10,952,852; 10,980,632; 10,980,633; 12,383,399 and US Pub. No. 2020/0405482, all of which are incorporated fully herein by reference. The frame material used to form the TAVR valve is typically TiAlV alloy, CoCr alloy or Nitinol™. The vast majority of cardiovascular implants include valves that are made at least in part using a CoCr alloy or Nitinol materials for construction of the structural frame of the valve.

A TAVR valve is designed to be compressed into a small diameter catheter, remotely placed within a patient's diseased aortic valve to take over the function of the native valve. Some TAVR valves are balloon-expandable, while others are self-expandable. In both cases, the TAVR valve is deployed within a calcified native valve that is forced permanently open and becomes the surface against which the frame is held in place by friction. A prosthetic heart valve can also be used to replace failing bioprosthetic or transcatheter valves, commonly known as a valve in valve procedure. Major TAVR advantages to the traditional surgical approaches include refraining from cardiopulmonary bypass, aortic cross-clamping and sternotomy which significantly reduces patients' morbidity.

However, several complications are associated with current TAV devices such as serious vascular injury or bleeding due to the large delivery profiles, mispositioning, crimp-induced leaflet damage, paravalvular leak, thrombosis, conduction system abnormalities and prosthesis-patient mismatch.

TAVR involves delivery, deployment, and implantation of a crimped, framed valve within a diseased aortic valve or degenerated bioprosthesis. Some limitation of the current procedure for TAVR include a) vascular complications such as dissection or severe bleeding due to the large size of the delivery system, b) recoil associated with the valve frame as defined as the frame being opened to a certain positional diameter and then relaxing or settling to a smaller diameter post balloon deflation which can lead to valve embolization, paravalvular leak, reduced effective orifice area (EOA), c) high incidence of conduction system injury leading to permanent pacemaker implantation or sudden cardiac death; the conduction abnormalities are worsened by the frame recoil which necessitates that the operator reach a higher balloon inflation diameter to obtain a physiologic effective orifice area after balloon deflation, d) longitudinal foreshortening of the frame during balloon expansion of the frame which can lead to mispositioning of the valve in the aortic annulus, e) imprecise alignment of the TAVR frame commissures with the native commissures which adversely affects hemodynamic performance and prosthetic valve durability, f) non-uniform valve frame expansion which leads to a non-cylindrical prosthetic valve which leads to increased acute and chronic complications such as leaflet thrombosis and structural valve deterioration, and g) high nickel content which is a common allergen.

Current structural heart valve procedures are limited by the combination of material and geometry of the prosthetic heart valve frame. The deficiencies include a) vascular complications such as dissection or severe bleeding due to the large size of the delivery system, b) neurological complications such as stroke due to the large size of the delivery system passing through calcified anatomy, c) adequate radial strength to restore physiological EOA in a diseased valve, while maintaining a crimp diameter for vascular access, d) recoil associated with the valve frame as defined as the frame being opened to a certain positional diameter and then relaxing or settling to a smaller diameter post balloon deflation which can lead to valve embolization, paravalvular leak, reduced effective orifice area (EOA), e) high incidence of conduction system injury leading to permanent pacemaker implantation or sudden cardiac death; the conduction abnormalities are worsened by the frame recoil which necessitates that the operator reach a higher balloon inflation diameter to obtain a physiologic effective orifice area after balloon deflation, f) foreshortening of the frame during balloon expansion of the frame which can lead to mispositioning of the valve in the aortic annulus, g) imprecise alignment of the prosthetic heart calve frame commissures with the native commissures which adversely affects hemodynamic performance, coronary blood flow, and prosthetic valve durability, h) acute coronary obstruction and coronary access impairment for re-intervention due to frame height, commissural misalignment, and malalignment of open cell geometry at the location of the coronaries, i) difficulties in later intervention of valve in valve due to valve height and/or misalignment of the prosthetic heart valve frame commissures with the native commissures, and putting patient at risk for coronary obstruction and coronary access impairment and overlap of open cells; cell size, etc., j) non-uniform valve frame expansion which leads to a non-cylindrical prosthetic valve which leads to increased acute and chronic complications such as leaflet thrombosis and structural valve deterioration, and k) high nickel content which is a common allergen.

In view of the current state of the art of prosthetic heart valves, there is a need for an improved prosthetic heart valve that addresses the above deficiencies.

### SUMMARY OF THE DISCLOSURE

The present disclosure is directed to a medical devices and medical device applications. The medical device can include, but is not limited to, a PFO (patent foramen ovale) device; stent (e.g., stent for used in aortic, iliac, subclavian, carotid, femoral artery, tibial, intracranial arteries, etc.); aneurysm exclusion devices (e.g., devices for aneurysm for use in aorta, iliac, intracranial arteries, etc.); valve (e.g., heart valve, TAVR valve, aortic, mitral valve replacement, tricuspid valve replacement, pulmonary valve replacement, etc.); anchoring devices for valves (e.g., anchoring devices for heart valve, TAVR valve, aortic valve, mitral valve, tricuspid valve, pulmonary valve, etc.); filters and structural features for valves, valve frames; occluders (e.g., occluders for patent foramen ovale, ventricular septal defect, left atrial appendage, etc.); guide wire; vascular implant; graft; guide wire; sheath, expandable sheath; catheter; needle; stent catheter; electrophysiology catheter; hypotube; staple; cutting device; pacemaker; dental implant; dental crown; dental braces; wire used in medical procedures; spinal implant; spinal discs; frame and other structure for use with a spinal implant; bone implant; artificial disk; artificial spinal disk; spinal interbody; expandable spinal interbody; interbody fusion device; expandable interbody fusion device; prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage; sutures; surgical staples; bone plate; knee replacement; hip replacement; shoulder replacement; ankle replacement; nail; rod; screw; post; cage; expandable cage; expandable orthopedic insert; plate (e.g., bone plate, cervical plate, spinal plate, etc.); bone plate nail; spinal rod; bone screw; post; spinal cage; pedicle screw; cap; hinge; joint system; screw extension; tulip extension; tether; graft; anchor; spacer; shaft; disk; ball; tension band; locking connector or other structural assembly that is used in a body to support a structure, mount a structure, and/or repair a structure in a body such as, but not limited to, a human body, animal body, etc. In one non-limiting embodiment, the medical device includes an expandable frame, more particularly the medical device is in the form of a cardiovascular implant for the treatment of structural heart disease wherein the cardiovascular implant includes an expandable frame, and still more particularly to a medical device is in the form of a prosthetic heart valve for the for the treatment of structural heart disease wherein the prosthetic heart valve includes an expandable frame that is formed of a rhenium alloy and/or a hafnium alloy and/or a refractory metal alloy containing metal alloy. Although the medical device will be particularly discussed with reference to a prosthetic heart valve, it will be appreciated by one skilled in the art that several of the features discussed herein such as to, but limited to, alloy composition, coatings on one or more portions of the medical device, alloy processing methods, processing methods to form all or a portion of the medical device, etc. can be used with other types of medical devices.

In one non-limiting aspect of the present disclosure, the use of a rhenium alloy and/or a hafnium alloy and/or a refractory metal alloy containing metal alloy to partially or fully form the frame of the prosthetic heart valve allows for a structural prosthetic heart valve frame geometry. The combination of the of the rhenium alloy and/or the hafnium alloy and/or the refractory metal alloy used to form the frame and the geometry of the frame of the prosthetic heart valve addresses the current deficiencies of prosthetic heart valves that are discussed above. The geometry of the frame of the prosthetic heart valve in combination with the frame being partially (e.g., 10-99.99 wt. % and all values and ranges therebetween) or fully formed of the rhenium alloy and/or the hafnium alloy and/or the refractory metal alloy containing metal alloy containing alloy enable the formation of an expandable frame that a) has an open cell geometry (i.e., an open cell is a cell in an frame that is not all formed by struts of the frame) that can be used to reduce delivery system size thereby reducing vascular and neurological complications, b) has an open cell pattern that has high radial strength due to the high yield strength and ultimate tensile strength of the rhenium containing metal alloy, c) has improved restoration of the physiologic EOA in challenging, heavily calcified valves that exert high force on the bioprosthetic valve, while also allowing a reduced crimp diameter for vascular access, d) has improved restoration of the physiologic EOA that results in greater longevity of the bioprosthetic valve, e) has lower recoil than traditional materials used to form frames such as stainless steel, chromium-cobalt, or titanium alloys, thereby resulting in less recoil of the frame when expanded which leads to decreased risk of valve embolization, decreased paravalvular leak due to improved conformability of the native anatomy, more accurate restoration of the physiologic EOA, and decrease conduction system injury due to a lower balloon inflation diameter required to obtain the physiologic EOA after balloon inflation, f) has an open cell geometry that is configured to have little or no (e.g., 0-20% longitudinal foreshortening along a longitudinal axis of the expandable frame and all values and ranges therebetween) or no foreshortening when expanded, which allows for more accurate placement of the valve in the native annulus, and wherein a frame that has little or no longitudinal foreshortening when expanded can be expanded with a shorter balloon, which use of a shorter balloon for frame expansion can decrease conduction system injury, g) has commissural alignment markers and an open cell between the commissures that allows for proper placement of the bioprosthetic valve in relation to the native commissures of the valve for proper hemodynamic function in regard to wash out of the valve and blood flow to the coronaries, which leads to better durability and longevity of the valve, and access and re-intervention of the coronaries preventing future adverse events, h) has an open cell geometry with radial symmetry, longitudinal symmetry, and little or no longitudinal foreshortening which allows for symmetrical and cylindrical expansion of the prosthetic valve resulting in lower rates of leaflet thrombosis and structural valve deterioration, i) is formed of a rhenium alloy and/or a hafnium alloy and/or a refractory metal alloy with no nickel content so as to prevent allergic response due to the presence of nickel and restenosis associated with nickel content, j) is formed of a rhenium alloy and/or a hafnium alloy and/or a refractory metal alloy that reduces adverse tissue reactions after implant of the medical device, k) is formed of a rhenium alloy and/or a hafnium alloy and/or a refractory metal alloy that reduces metal ion release after implant of the medical device, 1) is formed of a rhenium alloy and/or a hafnium alloy and/or a refractory metal alloy that reduces corrosion of the medical device after implant of the medical device, m) is formed of a rhenium alloy and/or a hafnium alloy and/or a refractory metal alloy that reduces allergic reaction after implant of the medical device, n) is formed of a rhenium alloy and/or a hafnium alloy and/or a refractory metal alloy that improves hydrophilicity of the medical device, n) is formed of a rhenium alloy and/or a hafnium alloy and/or a refractory metal alloy that lowers ion release from medical device into tissue, o) is formed of a rhenium alloy and/or a hafnium alloy and/or a refractory metal alloy that reduces toxicity of the medical device after implant of the medical device, p) includes an enhancement coating that can inhibit or prevent calcium deposits on one or more portions of the medical device (e.g., inhibit or prevent calcium deposits on frame, leaflets, skirt, etc.) so as to i) extend the life of the medical device, and/or ii) inhibit or prevent interference with the proper operation of the medical device, q) includes an enhancement coating that improve one or more properties of the metal alloy (e.g., change exterior color of metal alloy, increase hardness of coated surface, increase toughness of coated surface, reduced friction to coated surface, improve impact wear of coated surface, improve resistance to corrosion and oxidation, form a non-stick coated surface, improve biocompatibility of metal alloy having the coated surface, reduce toxicity of metal alloy having the coated surface, etc.), and/or r) includes an enhancement coating that that facilitates in the formation of i) nitric oxide (NO) production, ii) stimulation of endothelial cells, and/or iii) a modulation of endothelial cells.

In one non-limiting aspect of the disclosure, the prosthetic heart valve (e.g., heart valve, TAVR valve, mitral valve replacement, tricuspid valve replacement, pulmonary valve replacement, etc.) includes a radially collapsible and expandable frame and a leaflet structure that comprises a plurality of leaflets. In another non-limiting embodiment, the prosthetic heart valve optionally includes an annular or outer skirt that is disposed on and partially or fully covering or overlaid over the outer surface of the cells of at least a portion of the frame. In another non-limiting embodiment, the frame of the prosthetic heart valve comprises a plurality of interconnected vertically extending axial longitudinal member, angular articulating members and strut joints that define a plurality of open cells in the frame.

In another and/or alternative non-limiting aspect of the disclosure, the expandable frame of the prosthetic heart valve is optionally partially or fully formed of a metal alloy used to 1) increase the radiopacity of the medical device, 2) increase the radial strength of the medical device, 3) increase the yield strength and/or ultimate tensile strength of the medical device, 4) improve the stress-strain properties of the medical device, 5) improve the crimping and/or expansion properties of the medical device, 6) improve the bendability and/or flexibility of the medical device, 7) improve the strength and/or durability of the medical device, 8) increase the hardness of the medical device, 9) improve the recoil properties of the medical device, 10) improve the biostability and/or biocompatibility properties of the medical device, 11) increase fatigue resistance of the medical device, 12) resist cracking in the medical device and resist propagation of cracks, 13) enable smaller, thinner, and/or lighter weight medical device to be made, 14) reduce the outer diameter of a crimped medical device, 15) improve the conformity of the medical device to the shape of the treatment area when the medical device is used and/or expanded in the treatment area, 16) reduce the amount of recoil of the medical device to the shape of the treatment area when the medical device is expanded in the treatment area, 17) increase yield strength of the medical device, 18) improve fatigue ductility of the medical device, 18) improve durability of the medical device, 19) improve fatigue life of the medical device, 20) reduce adverse tissue reactions after implant of the medical device, 21) reduce metal ion release after implant of the medical device, 22) reduce corrosion of the medical device after implant of the medical device, 23) reduce allergic reaction after implant of the medical device, 24) improve hydrophilicity of the medical device, 25) reduce thickness of meta component of medical device, 26) improve bone fusion with medical device, 27) lower ion release from medical device into tissue, 28) reduce magnetic susceptibility of the medical device when implanted in a patient, and/or 29) reduce toxicity of the medical device after implant of the medical device. The metal alloy generally includes one or more materials that impart the desired properties to the medical device so as to withstand the manufacturing processes that are needed to produce the medical device. These manufacturing processes can include, but are not limited to, laser cutting, etching, crimping, annealing, drawing, pilgering, electroplating, electro-polishing, machining, plasma coating, 3D printing, 3D printed coatings, chemical vapor deposition, chemical polishing, cleaning, pickling, ion beam deposition or implantation, sputter coating, vacuum deposition, etc. In one non-limiting embodiment, the medical device is partially or fully formed by a 3D printing process.

In accordance with another and/or alternative non-limiting aspect of the present disclosure, the medical device can be formed of a variety of materials. In one non-limiting embodiment, the medical device is partially (e.g. 1-99.999 wt. % and all values and ranges therebetween) or fully formed of a metal alloy that includes a) stainless-steel, b) CoCr alloy, c) TiAIV alloy, d) aluminum alloy, e) nickel alloy, f) titanium alloy, g) tungsten alloy, h) molybdenum alloy, i) copper alloy, j) beryllium-copper alloy, k) titanium-nickel alloy, 1) refractory metal alloy, m) metal alloy (e.g., stainless-steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween), or n) metal alloy (e.g., stainless-steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) hafnium (e.g., 5-99 awt. % hafnium and all values and ranges therebetween). As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or 5-15 awt. % hafnium in the metal alloy is referred to as the “rhenium effect” or “hafnium effect.” As defined herein, a “rhenium effect” or a “hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative aspect of the present disclosure, the metal alloy used to partially or fully form the medical device includes a) stainless steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, 1) refractory metal alloy, m) metal alloy (e.g., stainless steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 atomic weight percent (awt. %) or atomic percent (awt. %) rhenium (e.g., 5-99 awt. % rhenium and all values and ranges therebetween), or n) metal alloy (e.g., stainless steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 atomic weight percent (awt. %) or atomic percent (awt. %) hafnium (e.g., 5-99 awt. % hafnium and all values and ranges therebetween).

In accordance with another and/or alternative non-limiting aspect of the present disclosure, the medical device can be formed of a variety of materials. In one non-limiting embodiment, the medical device is partially (e.g. 1-99.999 wt. % and all values and ranges therebetween) or fully formed of a metal alloy that includes a) stainless-steel, b) CoCr alloy, c) TiAlV alloy, d) aluminum alloy, e) nickel alloy, f) titanium alloy, g) tungsten alloy, h) molybdenum alloy, i) copper alloy, j) beryllium-copper alloy, k) titanium-nickel alloy, 1) refractory metal alloy, m) metal alloy (e.g., stainless-steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) rhenium (e.g., 5-99 awt. % rhenium and all values and ranges therebetween), or n) metal alloy (e.g., stainless-steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that is modified to further include at least 5 atomic weight percent (awt. %) or atomic percent (awt. %) hafnium (e.g., 5-99 awt. % hafnium and all values and ranges therebetween). As used herein, atomic weight percent (awt. %) or atomic percentage (awt %) or atomic percent (awt. %) are used interchangeably. As defined herein, the weight percentage (wt. %) of an element is the weight of that element measured in the sample divided by the weight of all elements in the sample multiplied by 100. The atomic percentage or atomic weight percent (awt. %) is the number of atoms of that element, at that weight percentage, divided by the total number of atoms in the sample multiplied by 100. The use of the terms weight percentage (wt. %) and atomic percentage or atomic weight percentage (awt. %) are two ways of referring to metallic alloy and its constituents. It has been found that for several metal alloys the inclusion of rhenium and/or hafnium results in the ductility and/or tensile strength of the metal alloy to improve as compared to a metal alloy is that absent rhenium and/or hafnium. Such improvement in ductility and/or tensile strength due to the inclusion of at least 5-15 awt. % rhenium and/or 5-15 awt. % hafnium in the metal alloy is referred to as the “rhenium effect” or “hafnium effect.” As defined herein, a “rhenium effect” or a “hafnium effect” is a) an increase of at least 10% in ductility of the metal alloy caused by the addition of rhenium and/or hafnium to the metal alloy, and/or b) an increase of at least 10% in tensile strength of the metal alloy caused by the addition of rhenium and/or hafnium to the metal alloy. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative aspect of the present disclosure, the metal alloy used to partially or fully form the medical device includes stainless-steel, CoCr alloys, TiAlV alloys, aluminum alloys, nickel alloys, titanium alloys, tungsten alloys, molybdenum alloys, copper alloys, MP35N alloys, or beryllium-copper alloys that have been modified to include at least 5-15 awt. % rhenium and/or 5-15 awt % hafnium so as to result in improved ductility and/or tensile strength as compared to the same metal alloy that is absent rhenium and/or hafnium. As defined herein, a stainless-steel alloy (SS alloy) includes at least 50 wt. % iron (e.g., 50-85 wt. % and all values and ranges therebetween), 10-30 wt. % chromium, 0-35 wt. % nickel, and optionally one or more of 0-5 wt. % molybdenum, 0-6 wt. % manganese, 0-1 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-72 wt. % cobalt, 15-35 wt. % chromium, and optionally one or more of 1-38 wt. % nickel, 2-18 wt. % molybdenum, 0-18 wt. % iron, 0-1 wt. % titanium, 0-2.8 wt. % manganese, 0-0.15 wt. % silver, 0-2 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox and Elgiloy alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium. As defined herein, a stainless-steel alloy (SS alloy) includes at least 50 wt. % iron (e.g., 50-85 wt. % and all values and ranges therebetween), 10-30 wt. % chromium, 0-35 wt. % nickel, and optionally one or more of 0-5 wt. % molybdenum, 0-6 wt. % manganese, 0-1 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, and 0-2 wt. % tungsten. A 316L alloy that falls within a stainless-steel alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a cobalt-chromium alloy (CoCr alloy) includes 30-72 wt. % cobalt, 15-35 wt. % chromium, and optionally one or more of 1-38 wt. % nickel, 2-18 wt. % molybdenum, 0-18 wt. % iron, 0-1 wt. % titanium, 0-2.8 wt. % manganese, 0-0.15 wt. % silver, 0-2 wt. % carbon, 0-16 wt. % tungsten, 0-2 wt. % silicon, 0-2 wt. % aluminum, 0-1 wt. % iron, 0-0.1 wt. % boron, 0-0.15 wt. % silver, and 0-2 wt. % titanium. As a MP35N alloy that falls within a CoCr alloy includes 18-22 wt. % chromium, 32-38 wt. % nickel, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. %, 0-0.1 wt. % boron, 0-0.15 wt. % silver, and balance cobalt. As defined herein, a Phynox™ and Elgiloy™ alloy that falls within a CoCr alloy includes 38-42 wt. % cobalt, 18-22 wt. % chromium, 14-18 wt. % iron, 13-17 wt. % nickel, 6-8 wt. % molybdenum. As defined herein, a L605 alloy that falls within a CoCr alloy includes 18-22 wt. % chromium, 14-16 wt. % tungsten, 9-11 wt. % nickel, balance cobalt. As defined herein, a titanium-aluminum-vanadium alloy (TiAlV alloy) includes 4-8 wt. % aluminum, 3-6 wt. % vanadium, 80-93 wt. % titanium, and optionally one or more of 0-0.4 wt. % iron, 0-0.2 wt. % carbon, 0-0.5 wt. % yttrium. A Ti-6Al-4V alloy that falls with a TiAlV alloy includes incudes 3.5-4.5 wt. % vanadium, 5.5-6.75 wt. % aluminum, 0.3 wt. % max iron, 0.08 wt. % max carbon, 0.05 wt. % max yttrium, balance titanium. As defined herein, an aluminum alloy includes 80-99 wt. % aluminum, and optionally one or more 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a nickel alloy includes 30-98 wt. % nickel, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon. As defined herein, a titanium alloy includes 80-99 wt. % titanium, and optionally one of more of 0-6 wt. % aluminum, 0-3 wt. % tin, 0-1 wt. % palladium, 0-8 wt. % vanadium, 0-15 wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a tungsten alloy includes 85-98 wt. % tungsten, and optionally one or more of 0-8 wt. % nickel, 0-5 wt. % copper, 0-5 wt. % molybdenum, 0-4 wt. % iron. As defined herein, a molybdenum alloy includes 90-99.5 wt. % molybdenum, and optionally one or more of 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a copper alloy includes 55-95 wt. % copper, and optionally one or more of 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20 wt. % nickel. As defined herein, a beryllium-copper alloy includes 95-98.5 wt. % copper, 1-4 wt. % beryllium, and optionally one or more of 0-1 wt. % cobalt, and 0-0.5 wt. % silicon. As defined herein, a titanium-nickel alloy (e.g., Nitinol alloy) includes 42-58 wt. % nickel and 42-58 wt. % titanium.

In accordance with another and/or alternative aspect of the present disclosure, the metal alloy used to partially or fully form the medical device includes at least 5 awt. % (e.g., 5-99 awt. % and all values and ranges therebetween) rhenium, and 0.1-96 wt. % (and all values and ranges therebetween) of one or more additives selected from the group of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a rhenium effect. In one non-limiting embodiment, the metal alloy is a stainless-steel alloy that has been modified to include at least 5-15 awt. % rhenium and/or hafnium. In another and/or alternative non-limiting embodiment, the metal alloy is a cobalt-chromium alloy that has been modified to include at least 5-15 awt. % rhenium and/or hafnium. In another and/or alternative non-limiting embodiment, the metal alloy is a TiAlV alloy that has been modified to include at least 5-15 awt. % rhenium and/or hafnium. In another and/or alternative non-limiting embodiment, the metal alloy is an aluminum alloy that has been modified to include at least 5-15 awt. % rhenium and/or hafnium. In another and/or alternative non-limiting embodiment, the metal alloy is a nickel alloy that has been modified to include at least 5-15 awt. % rhenium and/or hafnium. In another and/or alternative non-limiting embodiment, the metal alloy is a titanium alloy that has been modified to include at least 5-15 awt. % rhenium and/or hafnium. In another and/or alternative non-limiting embodiment, the metal alloy is a tungsten alloy that has been modified to include at least 15 awt. % rhenium and/or hafnium. In another and/or alternative non-limiting embodiment, the metal alloy is a molybdenum alloy that has been modified to include at least 5-15 awt. % rhenium and/or hafnium. In another and/or alternative non-limiting embodiment, the metal alloy is a copper alloy that has been modified to include at least 5-15 awt. % rhenium and/or hafnium. In another and/or alternative non-limiting embodiment, the metal alloy is a beryllium-copper alloy that has been modified to include at least 5-15 awt. % rhenium and/or hafnium.

In accordance with another and/or alternative aspect of the present disclosure, the metal alloy used to partially or fully form the medical device includes at least 5 awt. % (e.g., 5-99 awt. % and all values and ranges therebetween) hafnium, and 0.1-96 wt. % (and all values and ranges therebetween) of one or more additives selected from the group of aluminum, boron, beryllium, bismuth, cadmium, calcium, cerium, chromium, cobalt, copper, gallium, gold, iridium, iron, lanthanum, lithium, magnesium, manganese, molybdenum, nickel, niobium, osmium, palladium, platinum, rare earth metals, rhenium, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other components other than the additives (e.g., carbon, oxygen, phosphorous, sulfur, hydrogen, lead, nitrogen, etc.), and which metal alloy exhibits a hafnium effect. In one non-limiting embodiment, the metal alloy is a stainless-steel alloy that has been modified to include at least 5-15 awt. % hafnium. In another and/or alternative non-limiting embodiment, the metal alloy is a cobalt chromium alloy that has been modified to include at least 5-15 awt. % hafnium. In another and/or alternative non-limiting embodiment, the metal alloy is a TiAlV alloy that has been modified to include at least 5-15 awt. % hafnium. In another and/or alternative non-limiting embodiment, the metal alloy is an aluminum alloy that has been modified to include at least 5-15 awt. % hafnium. In another and/or alternative non-limiting embodiment, the metal alloy is a nickel alloy that has been modified to include at least 5-15 awt. % hafnium. In another and/or alternative non-limiting embodiment, the metal alloy is a titanium alloy that has been modified to include at least 5-15 awt. % hafnium. In another and/or alternative non-limiting embodiment, the metal alloy is a tungsten alloy that has been modified to include at least 5-15 awt. % hafnium. In another and/or alternative non-limiting embodiment, the metal alloy is a molybdenum alloy that has been modified to include at least 5-15 awt. % hafnium. In another and/or alternative non-limiting embodiment, the metal alloy is a copper alloy that has been modified to include at least 5-15 awt. % hafnium. In another and/or alternative non-limiting embodiment, the metal alloy is a beryllium-copper alloy that has been modified to include at least 5-15 awt. % hafnium.

In accordance with another and/or alternative aspect of the present disclosure, the metal alloy used to partially or fully form the medical device includes rhenium and/or hafnium, and molybdenum, and the weight percent of rhenium and/or hafnium in the metal alloy is optionally greater than the weight percent of molybdenum in the metal alloy, and the weight percent of one or more additive (e.g., aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium) in the metal alloy is optionally greater that the weight percent of molybdenum in the metal alloy, and the metal alloy optionally includes 0-2 wt. % of a combination of other components other than the additives (e.g., carbon, oxygen, phosphorous, sulfur, hydrogen, lead, nitrogen, etc.). In one non-limiting embodiment, the metal alloy is fully formed of or includes rhenium and/or hafnium, and molybdenum, and the weight percent of rhenium and/or hafnium plus the combined weight percent of additives is greater than the weight percent of molybdenum, and the metal alloy optionally includes 0-2 wt. % of a combination of other components other than the additives (e.g., carbon, oxygen, phosphorous, sulfur, hydrogen, lead, nitrogen, etc.).

In accordance with another and/or alternative aspect of the present disclosure, the metal alloy used to partially or fully form the medical device includes rhenium and/or hafnium, and molybdenum, and one or more additives selected from bismuth, niobium, tantalum, tungsten, titanium, vanadium, chromium, manganese, yttrium, zirconium, technetium, ruthenium, rhodium, hafnium, osmium, copper, and iridium, and the atomic weight percent of rhenium and/or hafnium to the atomic weight percent of the combination of one or more of bismuth, niobium, tantalum, tungsten, titanium, vanadium, chromium, manganese, yttrium, zirconium, technetium, ruthenium, rhodium, hafnium, osmium, copper, and iridium is 0.4:1 to 2.5:1 (and all values and ranges therebetween).

In accordance with another and/or alternative aspect of the present disclosure, the metal alloy used to partially or fully form the medical device includes at least 5 awt. % (e.g., 5-99 awt. % and all values and ranges therebetween) rhenium and/or hafnium plus at least two metals selected from the group of molybdenum, bismuth, chromium, iridium, niobium, tantalum, titanium, yttrium, and zirconium, and the content of the metal alloy that includes other elements and compounds is 0-0.1 wt. %. In another and/or alternative non-limiting embodiment, the metal alloy includes rhenium and/or hafnium, molybdenum, and chromium. In another and/or alternative non-limiting embodiment, the metal alloy includes at least 35 wt. % (e.g., 35-75 wt. % and all values and ranges therebetween) rhenium and/or hafnium, and the metal alloy also includes chromium. In one non-limiting embodiment, the metal alloy includes at least 35 wt. % rhenium and/or hafnium, and at least 25 wt. % (e.g., 25-49.9 wt. % and all values and ranges therebetween) of the metal alloy includes chromium, and optionally 0.1-40 wt. % (and all values and ranges therebetween) of the metal alloy includes one or more of aluminum, boron, beryllium, bismuth, cadmium, calcium, cerium, chromium, cobalt, copper, gallium, gold, hafnium, iridium, iron, lanthanum, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen. In another and/or alternative non-limiting embodiment, the metal alloy includes 15-50 awt. % rhenium and/or hafnium (and all values and ranges therebetween) and 0.5-70 awt. % chromium (and all values and ranges therebetween). In another and/or alternative non-limiting embodiment, the metal alloy includes 15-50 awt. % rhenium and/or hafnium (and all values and ranges therebetween) and 0.5-70 awt. % tantalum (and all values and ranges therebetween). In another and/or alternative non-limiting embodiment, the metal alloy includes 15-50 awt. % rhenium and/or hafnium (and all values and ranges therebetween) and 0.5-70 awt. % niobium (and all values and ranges therebetween). In another and/or alternative non-limiting embodiment, the metal alloy includes 15-50 awt. % rhenium and/or hafnium (and all values and ranges therebetween) and 0.5-70 awt. % titanium (and all values and ranges therebetween). In another and/or alternative non-limiting embodiment, the metal alloy includes 15-50 awt. % rhenium and/or hafnium (and all values and ranges therebetween) and 0.5-70 awt. % zirconium (and all values and ranges therebetween). In another and/or alternative non-limiting embodiment, the metal alloy includes 15-50 awt. % rhenium and/or hafnium (and all values and ranges therebetween) and 0.5-70 awt. % molybdenum (and all values and ranges therebetween). In another and/or alternative non-limiting embodiment, the metal alloy includes at least 15 awt. % rhenium and/or hafnium, greater than 50 wt. % titanium (e.g., 51-80 wt. % and all values and ranges therebetween), 15-45 wt. % (and all values and ranges therebetween) niobium, 0-10 wt. % (and all values and ranges therebetween) zirconium, 0-15 wt. % (and all values and ranges therebetween) tantalum, and 0-8 wt. % molybdenum (and all values and ranges therebetween).

In accordance with another and/or alternative aspect of the present disclosure, the metal alloy used to partially or fully form the medical device includes a refractory metal alloy, and wherein the refractory metal alloy includes at least 20 wt. % of one or more of niobium, rhenium, tantalum, molybdenum or tungsten (e.g., 20-99.9 wt. % and all values and ranges therebetween), and 0.1-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the medical device includes a refractory metal alloy, and wherein the refractory metal alloy includes at least 5 awt. % rhenium and/or hafnium, at least 20 wt. % of one or more of niobium, tantalum, molybdenum or tungsten (e.g., 20-99.9 wt. % and all values and ranges therebetween), and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the medical device includes a refractory metal alloy, and wherein the refractory metal alloy includes at least 20 wt. % of one or more of niobium, tantalum or tungsten, and wherein the refractory metal alloy includes 0-30 wt. % molybdenum (and all values and ranges therebetween), and wherein the refractory metal alloy includes at least 5 awt. % rhenium and/or hafnium (e.g., 5-80 awt. % rhenium and/or hafnium and all values and ranges therebetween), and wherein the refractory metal alloy includes and 0-80 wt. % (and all values and ranges therebetween) of one or more of calcium, carbon, cerium oxide, chromium, cobalt, copper, gold, hafnium, iridium, iron, lanthanum, lanthanum oxide, magnesium, manganese, molybdenum, nickel, osmium, platinum, rare earth metals, rhodium, ruthenium, silver, tantalum, technetium, titanium, vanadium, yttrium, yttrium oxide, zinc, zirconium, zirconium oxide, and/or alloys of one or more of such components.

In accordance with another and/or alternative aspect of the present disclosure, the metal alloy used to partially or fully form the medical device includes at least 5 awt. % rhenium and/or hafnium (e.g., 5-99 awt. % rhenium and/or hafnium and all values and ranges therebetween), and at least 0.1 wt. % of one or more additive metals selected from aluminum, bismuth, chromium, cobalt, copper, hafnium, iridium, iron, magnesium, manganese, nickel, niobium, osmium, rhodium, ruthenium, silicon, silver, tantalum, technetium, titanium, tungsten, vanadium, yttrium, and zirconium, and wherein the metal alloy includes 0-30 wt. % molybdenum (and all values and ranges therebetween), and wherein a combined weight percent of rhenium and/or hafnium, and the additive metals is 70-100 wt. % (and all values and ranges therebetween).

In accordance with another and/or alternative aspect of the present disclosure, the metal alloy used to partially or fully form the medical device includes stainless-steel that has been modified with at least 5 awt. % rhenium and/or hafnium (e.g., 5-50 awt. % rhenium and/or hafnium and all values and ranges therebetween), and wherein a combined weight percent of iron, chromium, nickel, tantalum, niobium, copper, manganese, aluminum, titanium, selenium, vanadium, tungsten, hafnium and rhenium is 70-100 wt. % (and all values and ranges therebetween).

In accordance with another and/or alternative aspect of the present disclosure, the metal alloy used to partially or fully form the medical device includes cobalt-chromium alloy that has been modified with at least 5 awt. % rhenium and/or hafnium (e.g., 5-50 awt. % rhenium and/or hafnium and all values and ranges therebetween), and wherein a combined weight percent of cobalt, chromium, nickel, iron, titanium, manganese, silver, tungsten, silicon, aluminum, iron, boron, silver, titanium, hafnium, and rhenium is 70-100 wt. % (and all values and ranges therebetween).

In accordance with another and/or alternative aspect of the present disclosure, the metal alloy used to partially or fully form the medical device includes titanium-aluminum-vanadium alloy that has been modified with at least 5 awt. % rhenium and/or hafnium (e.g., 5-50 awt. % rhenium and/or hafnium and all values and ranges therebetween), and wherein a combined weight percent of aluminum, vanadium, titanium, iron, yttrium, hafnium, and rhenium is 70-100 wt. % (and all values and ranges therebetween).

In accordance with another and/or alternative aspect of the present disclosure, the metal alloy used to partially or fully form the medical device includes aluminum alloy that has been modified with at least 5 awt. % rhenium and/or hafnium (e.g., 5-50 awt. % rhenium and/or hafnium and all values and ranges therebetween), and wherein a combined weight percent of aluminum, silicon, magnesium, manganese, scandium, beryllium, yttrium, cerium, chromium, iron, zinc, titanium, lithium, silver, calcium, zirconium, cadmium, bismuth, nickel, vanadium, gallium, copper, hafnium, and rhenium is 70-100 wt. % (and all values and ranges therebetween).

In accordance with another and/or alternative aspect of the present disclosure, the metal alloy used to partially or fully form the medical device includes nickel alloy that has been modified with at least 5 awt. % rhenium and/or hafnium (e.g., 5-50 awt. % rhenium and/or hafnium and all values and ranges therebetween), and wherein a combined weight percent of nickel, chromium, iron, copper, cobalt, aluminum, tantalum, tungsten, titanium, niobium, silicon, hafnium, and rhenium is 70-100 wt. % (and all values and ranges therebetween).

In accordance with another and/or alternative aspect of the present disclosure, the metal alloy used to partially or fully form the medical device includes titanium alloy that has been modified with at least 5 awt. % rhenium and/or hafnium (e.g., 5-50 awt. % rhenium and/or hafnium and all values and ranges therebetween), and wherein a combined weight percent of titanium, aluminum, tin, palladium, vanadium, nickel, ruthenium, chromium, zirconium, niobium, silicon, cobalt, iron, hafnium, and rhenium is 70-100 wt. % (and all values and ranges therebetween).

In accordance with another and/or alternative aspect of the present disclosure, the metal alloy used to partially or fully form the medical device includes tungsten alloy that has been modified with at least 5 awt. % rhenium and/or hafnium (e.g., 5-50 awt. % rhenium and/or hafnium and all values and ranges therebetween), and wherein a combined weight percent of tungsten, nickel, copper, iron, hafnium, and rhenium is 70-100 wt. % (and all values and ranges therebetween).

In accordance with another and/or alternative aspect of the present disclosure, the metal alloy used to partially or fully form the medical device includes copper alloy that has been modified with at least 5 awt. % rhenium and/or hafnium (e.g., 5-50 awt. % rhenium and/or hafnium and all values and ranges therebetween), and wherein a combined weight percent of copper, zinc, tin, iron, silicon, manganese, aluminum, beryllium, cobalt, nickel, hafnium, and rhenium is 70-100 wt. % (and all values and ranges therebetween).

In accordance with another and/or alternative aspect of the present disclosure, the metal alloy used to partially or fully form the medical device includes beryllium-copper alloy that has been modified with at least 5 awt. % rhenium and/or hafnium (e.g., 5-50 awt. % rhenium and/or hafnium and all values and ranges therebetween), and wherein a combined weight percent of copper, beryllium, cobalt, silicon, hafnium, and rhenium is 70-100 wt. % (and all values and ranges therebetween).

In accordance with another and/or alternative aspect of the present disclosure, the metal alloy used to partially or fully form the medical device includes titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In accordance with another and/or alternative aspect of the present disclosure, the metal alloy used to partially or fully form the medical device includes a metal alloy that includes less than 5 wt. % nickel (e.g., 0-4.99 wt. % and all values and ranges therebetween).

In accordance with another and/or alternative aspect of the present disclosure, the metal alloy used to partially or fully form the medical device includes a metal alloy that includes less than 5 wt. % chromium (e.g., 0-4.99 wt. % and all values and ranges therebetween).

In accordance with another and/or alternative aspect of the present disclosure, the metal alloy used to partially or fully form the medical device includes a primary metal (5-95 wt. % primary metal and all values and ranges therebetween) selected from one or more of molybdenum, rhenium, hafnium, niobium, tantalum, tungsten, and one or more alloying agents such as, but are not limited to, calcium, carbon, cerium oxide, chromium, cobalt, copper, gold, hafnium, iron, lanthanum oxide, magnesium, nickel, osmium, platinum, rare earth metals, rhenium, silver, technetium, titanium, vanadium, yttrium, yttrium oxide, zinc, zirconium, zirconium oxide, and/or alloys of one or more of such components (e.g., MoHfC, MoY2O3, MoCs2O, MoW, MoTa, MoZrO2, MoLa2O3, MoRe alloy, MoReW alloy, HfMo alloy, HfW alloy, ReW alloy, etc.).

In accordance with another and/or alternative aspect of the present disclosure, the metal alloy used to partially or fully form the medical device includes tungsten and copper and optionally one or more metal agents such as, but are not limited to, calcium, carbon, cerium oxide, chromium, cobalt, gold, hafnium, iron, lanthanum oxide, magnesium, molybdenum, nickel, niobium, osmium, platinum, rare earth metals, rhenium, silver, tantalum, technetium, titanium, vanadium, yttrium, yttrium oxide, zinc, zirconium, zirconium oxide, and/or alloys of one or more of such components. In one non-limiting formulation, the metal alloy includes 1-99.9 wt. % tungsten (and all values and ranges therebetween) (e.g., 1 wt. %, 1.01 wt. %, 1.02 wt. % . . . 99.88 wt. %, 99.89 wt. %, 99.9 wt. %), and 0.1-99 wt. % copper (and all values and ranges therebetween) (e.g., 0.1 wt. %, 0.101 wt. %, 0.102 wt. % . . . 98.998 wt. %, 98.999 wt. %, 99 wt. %). In another non-limiting formulation, the tungsten constitutes the greatest weight percent in the metal alloy and the copper constitutes the second greatest weight percent in the metal alloy. In another non-limiting formulation, the tungsten constitutes the largest weight percent of any component that forms the metal alloy. In another non-limiting formulation, the tungsten constitutes greater than 50 wt. % of the metal alloy.

In accordance with another and/or alternative aspect of the present disclosure, the metal alloy used to partially or fully form the medical device includes tungsten and rhenium and/or hafnium, and optionally one or more alloying agents such as, but not limited to, calcium, carbon, cerium oxide, chromium, cobalt, copper, gold, hafnium, iron, lanthanum oxide, magnesium, molybdenum, nickel, niobium, osmium, platinum, rare earth metals, rhenium, silver, tantalum, technetium, titanium, tungsten, vanadium, yttrium, yttrium oxide, zinc, zirconium, zirconium oxide, and/or alloys of one or more of such components (e.g., WRe, WReMo, WHf, WHfMo, WHfRe, WHfReMo, etc.). In one non-limiting formulation, the metal alloy includes 1-40 wt. % rhenium and/or hafnium (and all values and ranges therebetween and 60-99 wt. % tungsten (and all values and ranges therebetween). The total weight percent of the tungsten, rhenium and hafnium in the metal alloy is at least about 95 wt. % (e.g., 95-100% and all values and ranges therebetween). In another non-limiting formulation, the metal alloy includes 1-47.5 wt. % rhenium and/or hafnium (and all values and ranges therebetween) and 20-80 wt. % tungsten (and all values and ranges therebetween) and 0-47.5 wt. % molybdenum (and all values and ranges therebetween). The total weight percent of the tungsten, molybdenum, rhenium and hafnium in the metal alloy is at least about 95 wt. % (e.g., 95-100% and all values and ranges therebetween). In one non-limiting specific metal alloy, the weight percent of the tungsten is greater than a weight percent of rhenium, hafnium and/or molybdenum. In another non-limiting specific metal alloy, the weight percent of the tungsten is greater than 50 wt. % of the metal alloy. In another non-limiting specific metal alloy, the weight percent of the tungsten is greater than a weight percent of rhenium and/or hafnium, but is less than a weigh percent of molybdenum. In another non-limiting specific metal alloy, the weight percent of the tungsten is greater than a weight percent of molybdenum, but less than a weigh percent of rhenium and/or hafnium. In another non-limiting metal alloy, the weight percent of the tungsten is less than a weight percent of rhenium, hafnium and/or molybdenum.

In accordance with another and/or alternative aspect of the present disclosure, the metal alloy used to partially or fully form the medical device includes a metal alloy that has an atomic weight percent of rhenium and/or hafnium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium in the metal alloy is 0.7:1 to 1.5:1 (and all values and ranges therebetween).

In accordance with another and/or alternative aspect of the present disclosure, the metal alloy used to partially or fully form the medical device includes two of bismuth, niobium, tantalum, tungsten, titanium, vanadium, chromium, manganese, yttrium, zirconium, technetium, ruthenium, rhodium osmium, copper, and iridium, the atomic ratio of the two metals is 0.4:1 to 2.5:1 (and all values and ranges therebetween).

In accordance with another and/or alternative aspect of the present disclosure, the metal alloy used to partially or fully form the medical device includes a titanium-nickel alloy that has been modified with at least 5 awt. % rhenium and/or hafnium (e.g., 5-50 awt. % rhenium and/or hafnium and all values and ranges therebetween), and wherein a combined weight percent of nickel, titanium, hafnium, and rhenium is 70-100 wt. % (and all values and ranges therebetween).

In accordance with another and/or alternative non-limiting aspect of the present disclosure, at least 10 wt. % (e.g., 10-95 wt. % and all values and ranges therebetween) of the metal alloy that includes at least 15 atw. % hafnium and/or rhenium and also includes one or more of molybdenum, niobium, tantalum, or tungsten.

In accordance with another and/or alternative non-limiting aspect of the present disclosure, there is provided a metal alloy that includes at least 15 atw. % hafnium and/or rhenium and 0.1-75 wt. % (and all values and ranges therebetween) of one or more of aluminum, bismuth, calcium, carbon, chromium, cobalt, copper, gold, iridium, iron, lanthanum, magnesium, manganese, molybdenum, nickel, niobium, osmium, platinum, rare earth metals, rhodium, ruthenium, silver, tantalum, technetium, titanium, tungsten, vanadium, yttrium, zinc, zirconium, and/or alloys of one or more of such components.

In another non-limiting aspect of the present disclosure, the metals used to form the metal alloy includes at least 15 atw. % hafnium and/or rhenium, nickel and tungsten and optionally one or more alloying agents such as, but not limited to, aluminum, bismuth, calcium, carbon, chromium, cobalt, copper, gold, iron, magnesium, molybdenum, niobium, osmium, platinum, rare earth metals, rhenium, silver, tantalum, technetium, titanium, vanadium, yttrium, zinc, zirconium, and/or alloys of one or more of such components (e.g., WNi, WNiMo, WNi.Re, etc.). In another non-limiting formulation, the metal alloy that includes at least 15 atw. % hafnium and/or rhenium includes 1-47.5 wt. % nickel (and all values and ranges therebetween) and 20-80 wt. % tungsten (and all values and ranges therebetween) and 1-47.5 wt. % (and all values and ranges therebetween) of one or more of aluminum, bismuth, calcium, carbon, chromium, cobalt, copper, gold, hafnium, iron, magnesium, molybdenum, nickel, niobium, osmium, platinum, rare earth metals, silver, tantalum, technetium, titanium, vanadium, yttrium, zinc, zirconium.

In accordance with another and/or alternative non-limiting aspect of the present disclosure, the metal alloy includes at least 15 atw. % hafnium and/or rhenium and nickel.

In accordance with another and/or alternative aspect of the present disclosure, the metal alloy includes at least 15 atw. % hafnium and/or rhenium includes (e.g., 15-99 awt. % and all values and ranges therebetween), optionally 5 awt. % (e.g., 5-99 awt. % and all values and ranges therebetween) molybdenum, optionally 5 awt. % (e.g., 5-99 awt. % and all values and ranges therebetween) niobium, optionally 5 awt. % (e.g., 5-99 awt. % and all values and ranges therebetween) tantalum, optionally 5 awt. % (e.g., 5-99 awt. % and all values and ranges therebetween) tungsten, and at least 0.1 wt. % (e.g., 0.1 wt. % to 96 wt. % and all values and ranges therebetween) of one or more of aluminum, boron, beryllium, bismuth, cadmium, calcium, cerium, chromium, cobalt, copper, gallium, gold, iridium, iron, lanthanum, lithium, magnesium, manganese, nickel, osmium, palladium, platinum, rare earth metals, rhodium, ruthenium, scandium, silver, silicon, technetium, tin, titanium, vanadium, yttrium, zinc, and/or zirconium, and the metal alloy optionally includes 0-2 wt. % (and all values and ranges therebetween) of a combination of other metals, carbon, oxygen, phosphorous, sulfur, hydrogen and/or nitrogen.

In accordance with another and/or alternative non-limiting aspect of the present disclosure, there is provided a medical device or other type of device partially or fully formed of a metal alloy that includes at least 15 atw. % hafnium. In one non-limiting embodiment, 50-100% (and all values and ranges therebetween) of the medical device or other type of device is formed of the metal alloy that includes at least 15 atw. % hafnium. In another non-limiting embodiment, at least 30 wt. % (e.g., 30-100 wt. % and all values and ranges therebetween) of the medical device or other type of device is formed of a metal alloy that includes at least 15 atw. % hafnium.

In accordance with another and/or alternative aspect of the present disclosure, the metal alloy used to partially or fully form the medical device includes less than 5 wt. % nickel (e.g., 0-4.99 wt. % and all values and ranges therebetween).

In accordance with another and/or alternative aspect of the present disclosure, the metal alloy used to partially or fully form the medical device includes less than 5 wt. % chromium (e.g., 0-4.99 wt. % and all values and ranges therebetween).

In accordance with another and/or alternative non-limiting aspect of the present disclosure, the metal alloy that that partially or fully forms the medical device is optionally subjected to one or more manufacturing processes. These manufacturing processes can include, but are not limited to, expansion, laser cutting, etching, crimping, annealing, drawing, pilgering, electroplating, electro-polishing, machining, plasma coating, 3D printing, 3D printed coatings, chemical vapor deposition, chemical polishing, cleaning, pickling, ion beam deposition or implantation, sputter coating, vacuum deposition, EDM cutting, gun-drilling, compression, sintering, compression process, consolidation process, etc.

In accordance with another and/or alternative non-limiting aspect of the present disclosure, the metal alloy that partially or fully forms the medical devise optionally has a generally uniform density throughout the metal alloy, and also results in the desired yield and ultimate tensile strengths of the metal alloy. In one non-limiting embodiment, the density of the metal alloy that includes at least 15 atw. % rhenium and/or hafnium and/or is a refractory metal alloy is generally at least about 5 gm/cc (e.g., 5 gm/cc-21 gm/cc and all values and ranges therebetween; 10-20 gm/cc; etc.), and typically at least about 11-19 gm/cc.

In accordance with another and/or alternative non-limiting aspect of the present disclosure, the metal alloy optionally includes a certain amount of carbon and oxygen; however, this is not required. The carbon to oxygen atomic ratio can be as low as about 0.2:1 (e.g., 0.2:1 to 50:1 and all values and ranges therebetween).

In accordance with another and/or alternative non-limiting aspect of the present disclosure, the metal alloy optionally includes a controlled amount of nitrogen; however, this is not required. In one non-limiting formulation, the metal alloy includes less than about 0.001 wt. % nitrogen (e.g., 0 wt. % to 0.0009999 wt. % and all values and ranges therebetween). In one non-limiting formulation of the metal alloy, the atomic ratio of carbon to nitrogen ion the metal alloy is at least about 1.5:1 (e.g., 1.5:1 to 400:1 and all values and ranges therebetween). In another non-limiting formulation of the metal alloy, the atomic ratio of oxygen to nitrogen is at least about 1.2:1 (e.g., 1.2:1 to 150:1 and all value and ranges therebetween).

In another and/or alternative non-limiting aspect of the present disclosure, the metal alloy 1) optionally is not clad, metal sprayed, plated, and/or formed (e.g., cold worked, hot worked, etc.) onto another metal, 2) optionally does not have another metal or metal alloy metal sprayed, plated, clad, and/or formed onto the metal alloy, 3) optionally is clad, metal sprayed, plated and/or formed (e.g., cold worked, hot worked, etc.) onto another metal, or 4) optionally has another metal or metal alloy metal sprayed, plated, clad and/or formed onto the metal alloy.

In accordance with another and/or alternative non-limiting aspect of the present disclosure, the medical device or other device can optionally be at least partially or fully formed from a tube or rod of metal alloy, or is formed into a shape that is at least 80% of the final net shape of the medical device (e.g., formed by 3D printing, formed by compression and/or sintering of metal alloy powder, formed by gun-drilling and/or EDM cutting, etc.). When the metal alloy is formed into a rod, the rod can optionally be gun-drilled and/or subjected to EDM cutting or otherwise cut to form a tube from the rod.

In accordance with another and/or alternative non-limiting aspect of the present disclosure, all or a portion of the medical device or other type of device that is formed of the metal alloy can be at least partially or fully formed from by 3D printing. As can be appreciated, other portions of the medical device or other type of device can be formed by 3D printing.

In accordance with another and/or alternative non-limiting aspect of the present disclosure, the metal alloy is optionally subjected to a swaging process; however, this is not required. In one non-limiting embodiment, swaging is performed on the metal alloy to at least partially or fully achieve final dimensions of one or more portions of the medical device or other type of device. Where there are undercuts of hollow structures in the medical device, which is not required, a separate piece of metal can be placed in the undercut to at least partially fill the gap. The separate piece of metal, when used, can be designed to be later removed from the undercut; however, this is not required. The swaging operation can be performed on the in areas of the metal alloy to be hardened. For a round or curved portion of a medical device or other device, the swaging can be rotary. For non-round portion of the medical device or other device, the swaging of the non-round portion can be performed by non-rotating swaging dies. The swaging temperature for a particular metal alloy can vary. In one non-limiting embodiment, when the swaging temperature is 10-400° C. and all values and ranges therebetween, the swaging can be conducted in air or an oxidizing environment. When the swaging temperature is increase above 400° C. (e.g., 400-1500+° C. and all values and ranges therebetween) or even when the swaging temperature is at or below 400° C., the swaging process can be performed in a controlled neutral or non-reducing environment (e.g., inert environment). The swaging process can be conducted by repeatedly hammering the metal alloy at the location to be hardened at the desired swaging temperature. In one non-limiting embodiment, during the swaging process, ions of boron and/or nitrogen are allowed to impinge upon atoms in the metal alloy. The medical device can optionally be swaged in multiple directions in a single operation or in multiple operations to achieve a hardness in desired location and/or direction of the medical device. The swaging temperature for a particular metal alloy (e.g., MoRe alloy, ReW alloy, HfW, etc.) can vary. For a refractory alloy (e.g., MoRe alloy, ReW alloy, Hf alloy, Mo alloy, Re alloy, W alloy, etc.) or a metal alloy that includes at least 15 atw. % rhenium and/or hafnium, the swaging temperature can be from room temperature (RT) (e.g., 10-27° C. and all values and ranges therebetween) to about 400° C. (e.g., 10-400° C. and all values and ranges therebetween) if the swaging is conducted in air or an oxidizing environment. The swaging temperature can be increased to up to about 1500° C. (e.g., 10-1500° C. and all values and ranges therebetween) if the swaging process is performed in a controlled neutral or non-reducing environment (e.g., inert environment). The swaging process can be conducted by repeatedly hammering the medical device at the location to be hardened at the desired swaging temperature. In one non-limiting embodiment, when the metal alloy includes rhenium and/or hafnium, during the swaging process ions of boron and/or nitrogen are allowed to impinge upon rhenium and/or hafnium atoms in the MoRe alloy or HfMo alloy or ReHfMo alloy so as to form ReB₂, ReN₂, ReN₃, HfB₂, HfN₂ and/or HrN₃; however, this is not required. It has been found that ReB₂, ReN₂, ReN₃, HfB₂, HfN₂ and/or HrN₃ are ultra-hard compounds. As can be appreciated, other refractory alloys that include Re and/or Hf and that are subjected to a swaging process can also form compounds that include boron and/or nitrogen. In one non-limiting process, the metal for the medical device can be machined and shape into the medical device when the metal is in a less hardened state. As such, the raw starting material can be first annealed to soften and then machined into the metal into a desired shape. After the metal alloy is shaped, the metal alloy can be re-hardened. The hardening of the metal material of the medical device can improve the wear resistance and/or shape retention of the medical device. The metal material of the medical generally cannot be re-hardened by annealing, thus a special rehardening processes is required. Such rehardening can be achieved by the swaging process of the present disclosure.

In accordance with another and/or alternative non-limiting aspect of the present disclosure, the metal alloy can optionally be nitrided; however, this is not required. The nitride layer on the metal alloy can function as a lubricating surface during the optional drawing of the metal alloy, and/or to form a metal alloy that has a smooth and/or lower friction outer surface. After the metal alloy is nitrided, the nitride metal alloy can optionally be cleaned; however, this is not required. The thickness of the nitrided surface layer is generally less than about 1 mm. In one non-limiting embodiment, the thickness of the nitrided surface layer is at least about 50 nanometer and less than about 1 mm (and all values and ranges therebetween). Generally, the weight percent of nitrogen in the nitrided surface layer is 0.0001-5 wt. % nitrogen (and all values and ranges therebetween). In one non-limiting embodiment, the weight percent of nitrogen in the nitrided surface layer is generally less than one of the primary components of the metal alloy, and typically less than each of the two primary components of the metal alloy. For a metal alloy that includes at least 15 atw. % hafnium and/or rhenium, or a refractory metal alloy, the nitride surface layer typically includes 0.001-5 wt. % nitrogen (and all values and ranges therebetween), and the primary constituents of the metal alloy that includes at least 15 atw. % hafnium and/or rhenium, or a refractory metal alloy (e.g., metals that constitute at least 5 wt. % of the metal alloy that includes at least 15 atw. % hafnium and/or rhenium, or the refractory metal alloy) are present in the nitride surface layer in a greater weight percent than the nitrogen content in the metal alloy. The nitriding process for the metal alloy can be used to a) form a lubricating surface, b) increase surface hardness and/or wear resistance of the metal alloy, and/or c) inhibit or prevent discoloration of the metal alloy (e.g., discoloration by oxidation, etc.).

In accordance with another and/or alternative non-limiting aspect of the present disclosure, the metal alloy, just prior to or after being partially or fully formed into the desired medical device or other type of device, can optionally be cleaned, polished, sterilized, nitrided, etc., for final processing of the metal alloy.

In accordance with another and/or alternative non-limiting aspect of the present disclosure, the use of the metal alloy that includes at least 15 atw. % hafnium and/or rhenium, or the refractory metal alloy to partially or fully form the medical device or other type of device can be used to increase the strength, hardness, and/or durability of the medical device or other type of device compared with the same or similar medical device or other type of device that is formed of stainless steel, chromium-cobalt alloys, or titanium alloys; thus, a lesser quantity of metal alloy can be used in the medical device or type of device to achieve similar strengths compared to medical devices or other type of device that is formed of different metals. As such, the resulting medical device or other type of device can be made smaller and/or less bulky by use of the metal alloy without sacrificing the strength and durability of the medical device or other type of device.

In another and/or alternative non-limiting aspect of the present disclosure, the metal alloy includes less than about 5 wt. % (e.g., 0-4.999999 wt. % and all values and ranges therebetween) other metals and/or impurities. A high purity level of the metal alloy results in the formation of a more homogeneous alloy, which in turn results in a more uniform density throughout the metal alloy, and also results in the desired yield and ultimate tensile strengths of the metal alloy. In one non-limiting composition, the metal alloy includes less than about 1 weight percent other metals and/or impurities. In another and/or alternative non-limiting composition, the metal alloy includes less than about 0.5 weight percent other metals and/or impurities. In still another and/or alternative non-limiting composition, the metal alloy includes less than about 0.4 weight percent other metals and/or impurities. In yet another and/or alternative non-limiting composition, the metal alloy includes less than about 0.2 weight percent other metals and/or impurities. In still yet another and/or alternative non-limiting composition, the metal alloy includes less than about 0.1 weight percent other metals and/or impurities. In a further and/or alternative non-limiting composition, the metal alloy includes less than about 0.05 weight percent other metals and/or impurities. In still a further and/or alternative non-limiting composition, the metal alloy includes less than about 0.02 weight percent other metals and/or impurities. In yet a further and/or alternative non-limiting composition, the metal alloy includes less than about 0.01 weight percent other metals and/or impurities. As can be appreciated, other weight percentages of the amount of other metals and/or impurities in the metal alloy can exist.

In another non-limiting aspect of the present disclosure, carbon nanotubes (CNT) can optionally be incorporated into a metal alloy that is used to at least partially form the medical device. The metal alloy can optionally include 0.05 wt. % CNT (e.g., 0.05 wt. % to 10 wt. % and all values and ranges therebetween).

In another and/or alternative non-limiting aspect of the present disclosure, the metal alloy (when used) can be used to form a coating on a portion or all of a medical device. For example, the metal alloy can be used as a coating on a polymer or base metal that is used to form all or a portion of a medical device (e.g., medical device for articulation points of artificial joints, etc.). Such coating can provide the benefit of improved wear, improved scratch resistance, improved hardness, and/or eliminate leaching of harmful metallic ions (i.e., Co, Cr, etc.) from the medical device (e.g., medical devices having articulating surfaces when they undergo fretting (i.e., scratching during relative motion)). As can be appreciated, the metal alloy can have other or additional advantages. As can also be appreciated, the metal alloy can be coated on other or additional types of medical devices. The coating process on a base metal (e.g., Ti or Ti alloy, Co or Co alloy, Cr alloy, CoCr alloy, stainless-steel, Fe and Fe alloys, Ni alloy, W alloy, Mo or Mo alloy, Hf or Hf alloy, Re or Re alloy, etc.) or polymer can be by plasma coating, chemical vapor deposition, and/or 3D printing of the coating on the base metal or polymer. The coating thickness of the metal alloy is non-limiting (e.g., 0.000001-0.5 inches and all values and ranges therebetween). In one non-limiting example, there is provided a medical device in the form of a clad rod wherein in the core of the rod is formed of a metal or ceramic or composite material or polymer, and the outer layer of the rod is formed of the metal alloy that was coated by a plasma coating process, a chemical vapor deposition process, or 3D printing process. The core and the outer coated layer of the rod can each form 50-99% (and all values and ranges therebetween) of the overall cross section of the rod. The base hardness of metal alloy can be as low as 300 Vickers and/or as high as 500 Vickers (e.g., 300-500 Vickers and all values and ranges therebetween). However, at high hardness the properties may not be desirable. In instances where the properties of fully annealed material is desired, but only the surface requires to be hardened as in this disclosure, the present disclosure includes a method that can provide benefits of both a softer metal alloy with a harder outer surface or shell. A non-limiting example is an orthopedic screw where a softer iron alloy is desired for high ductility as well as ease of machinability. Simultaneously, a hard shell is desired of the finished screw. While the inner hardness can range from 250 Vickers to 550 Vickers (and all values and ranges therebetween), the outer hardness can vary from 350 Vickers to 1000 Vickers (and all values and ranges therebetween) when using metal alloy.

In another and/or alternative non-limiting aspect of the present disclosure, the metal alloy can be used to form a core of a portion or all of a medical device. For example, a medical device can be in the form of a rod. The core of the rod can be formed of the metal alloy and then the outside of the core can then be coated with one or more other materials (e.g., another type of metal, polymer coating, ceramic coating, composite material coating, etc.). Such a rod can be used, for example, for orthopedic applications such as, but not limited to, spinal rods and/or pedicle screw systems. Non-limiting benefits to using the metal alloy in the core of a medical device can reducing the size of the medical device, increasing the strength of the medical device, and/or maintaining or reducing the cost of the medical device. As can be appreciated, the metal alloy can have other or additional advantages. As can also be appreciated, the metal alloy can form the core of other or additional types of medical devices. The core size and/or thickness of the metal alloy are non-limiting. In one non-limiting example, there is provided a medical device in the form of a clad rod wherein in the core of the rod is formed of a metal alloy, and the other layer of the clad rod is formed of a metal or metal alloy. The core and the other layer of the rod can each form 50-99% (and all values and ranges therebetween) of the overall cross section of the rod. As can also be appreciated, the metal alloy can form the core of other or additional types of medical devices.

In another and/or alternative non-limiting aspect of the present disclosure, the metal alloy has several physical properties that positively affect the medical device when the medical device is at least partially formed of the metal alloy of the present disclosure. In one non-limiting embodiment of the disclosure, the average Vickers hardness of metal alloy of the present disclosure used to form the medical device is generally at least about 234 DHP (Vickers) (i.e., Rockwell A hardness of at least about 60 at 77° F., Rockwell C hardness of at least about 19 at 77° F.) (e.g., 234 DPH to 700 DPH and all values and ranges therebetween; Rockwell C hardness of 19-60 at 77° F. and all values and ranges therebetween); however, this is not required. In one non-limiting aspect of this embodiment, the average hardness of the metal alloy of the present disclosure used to form the medical device is generally at least about 248 DHP (i.e., Rockwell A hardness of at least about 62 at 77° F., Rockwell C hardness of at least about 22 at 77° F.). In another and/or additional non-limiting aspect of this embodiment, the average hardness of the metal alloy of the present disclosure used to form the medical device is generally about 248-513 DHP (i.e., Rockwell A hardness of about 62-76 at 77° F., Rockwell C hardness of about 22-50 at 77° F.). In still another and/or additional non-limiting aspect of this embodiment, the average hardness of the metal alloy of the present disclosure used to form the medical device is generally about 272-458 DHP (i.e., Rockwell A hardness of about 64-74 at 77° F., Rockwell C hardness of about 26-46 at 77° F.). The metal alloy of the present disclosure generally has an average hardness that is greater than stainless-steel. In another and/or alternative non-limiting embodiment of the disclosure, the average ultimate tensile strength of the metal alloy of the present disclosure is generally at least about 60 UTS (Ksi); however, this is not required. In one non-limiting aspect of this embodiment, the average ultimate tensile strength of the metal alloy of the present disclosure is generally at least about 70 UTS (Ksi) (e.g., 70 UTS to 850 UTS and all values and ranges therebetween), and typically about 80-550 UTS (Ksi). The average ultimate tensile strength of the metal alloy of the present disclosure may vary somewhat when the metal alloy is in the form of a tube or a solid wire. When the metal alloy of the present disclosure is in the form of a tube, the average ultimate tensile strength of the metal alloy of the present disclosure is generally about 80-550 UTS (Ksi) (and all values and ranges therebetween), typically at least about 110 UTS (Ksi), and more typically 110-150 UTS (Ksi). When the metal alloy of the present disclosure is in the form of a solid wire, the average ultimate tensile strength of the metal alloy of the present disclosure wire is generally about 120-650 UTS (Ksi) (and all values and ranges therebetween). In still another and/or alternative non-limiting embodiment of the disclosure, the average yield strength of the metal alloy of the present disclosure is at least about 70 Ksi (e.g., 70-150 Ksi and all values and ranges therebetween); however, this is not required. In one non-limiting aspect of this embodiment, the average yield strength of the metal alloy of the present disclosure used to form the medical device is at least about 80 Ksi, and typically about 100-150 (Ksi). In yet another and/or alternative non-limiting embodiment of the disclosure, the average grain size of the metal alloy of the present disclosure used to form the medical device is no greater than about 4 ASTM (e.g., 4 ASTM to 20 ASTM using ASTM E112 and all values and ranges therebetween, e.g., 0.35 micron to 90 micron, and all values and ranges therebetween); however, this is not required. The grain size as small as about 14-15 ASTM can be achieved; however, the grain size is typically larger than 15 ASTM. The small grain size of the metal alloy of the present disclosure enables the medical device to have the desired elongation and ductility properties that are useful in enabling the medical device to be formed, crimped and/or expanded. In one non-limiting aspect of this embodiment, the average grain size of the metal alloy of the present disclosure used to form the medical device is about 5.2-10 ASTM, typically about 5.5-9 ASTM, more typically about 6-9 ASTM, still more typically about 6-9 ASTM, even more typically about 6.6-9 ASTM, and still even more typically about 7-8.5 ASTM; however, this is not required.

In another and/or alternative non-limiting embodiment of the disclosure, the average tensile elongation of the metal alloy of the present disclosure used to form the medical device is at least about 25% (e.g., 25%-50% average tensile elongation and all values and ranges therebetween). An average tensile elongation of at least 25% for the metal alloy is important to enable the medical device to be properly expanded when positioned in the treatment area of a body passageway. A medical device that does not have an average tensile elongation of at least about 25% can form micro-cracks and/or break during the forming, crimping and/or expansion of the medical device. In one non-limiting aspect of this embodiment, the average tensile elongation of the metal alloy of the present disclosure used to form the medical device is about 25-35%. The unique combination of the metals in the metal alloy of the present disclosure in combination with achieving the desired purity and composition of the alloy and the desired grain size of the metal alloy results in 1) a medical device having the desired high ductility at about room temperature, 2) a medical device having the desired amount of tensile elongation, 3) a homogeneous or solid solution of a metal alloy having high radiopacity, 4) a reduction or prevention of micro-crack formation and/or breaking of the metal alloy of the present disclosure tube when the tube is sized and/or cut to form the medical device, 5) a reduction or prevention of micro-crack formation and/or breaking of the medical device when the medical device is crimped onto a balloon and/or other type of medical device for insertion into a body passageway, 6) a reduction or prevention of micro-crack formation and/or breaking of the medical device when the medical device is bent and/or expanded in a body passageway, 7) a medical device having the desired ultimate tensile strength and yield strength, 8) a medical device that can have very thin wall thicknesses and still have the desired radial forces needed to retain the body passageway on an open state when the medical device has been expanded, and/or 9) a medical device that exhibits less recoil when the medical device is crimped onto a delivery system and/or expanded in a body passageway.

In another and/or alternative non-limiting aspect of the present disclosure, the use of the metal alloy to partially or fully form the medical device can be used to increase the strength and/or hardness and/or durability of the medical device as compared with stainless-steel or chromium-cobalt alloys; thus, less quantity of metal alloy can be used in the medical device to achieve similar strengths as compared to medical devices formed of different metals. As such, the resulting medical device can be made smaller and less bulky by use of the metal alloy without sacrificing the strength and durability of the medical device. Such a medical device can have a smaller profile, thus can be inserted in smaller areas, openings and/or passageways. The metal alloy can also increase the radial strength of the medical device. For instance, the thickness of the walls of the medical device and/or the wires used to form the medical device can be made thinner and achieve a similar or improved radial strength as compared with thicker walled medical devices formed of stainless-steel or cobalt and chromium alloy. The metal alloy also can improve stress-strain properties, bendability and flexibility of the medical device, thus increasing the life of the medical device. For instance, the medical device can be used in regions that subject the medical device to bending. Due to the improved physical properties of the medical device from the metal alloy, the medical device has improved resistance to fracturing in such frequent bending environments. In addition or alternatively, the improved bendability and flexibility of the medical device due to the use of the metal alloy can enable the medical device to be more easily inserted into various regions of a body. The metal alloy can also reduce the degree of recoil during the crimping and/or expansion of the medical device. For example, the medical device better maintains its crimped form and/or better maintains its expanded form after expansion due to the use of the metal alloy. As such, when the medical device is to be mounted onto a delivery device when the medical device is crimped, the medical device better maintains its smaller profile during the insertion of the medical device into various regions of a body. Also, the medical device better maintains its expanded profile after expansion so as to facilitate in the success of the medical device in the treatment area. In addition to the improved physical properties of the medical device by use of the metal alloy, the metal alloy has improved radiopaque properties as compared to standard materials such as stainless-steel or cobalt-chromium alloy, thus reducing or eliminating the need for using marker materials on the medical device. For instance, the metal alloy is believed to be at least about 10-20% more radiopaque than stainless-steel or cobalt-chromium alloy. Specifically, the metal alloy (e.g., refractory alloys) is believed to be at least about 33% more radiopaque than cobalt-chromium alloy and is believed to be at least about 40% more radiopaque than stainless-steel.

In accordance with another and/or alternative non-limiting aspect of the present disclosure, the metal alloy or medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. The type and/or amount of agent included in medical device and/or coated on medical device can vary. When two or more agents are included in and/or coated on medical device, the amount of two or more agents can be the same or different. The type and/or amount of agent included on, in and/or in conjunction with medical device are generally selected to address one or more clinical events. As defined herein, an agent is not an enhancement coating as defined herein.

In accordance with another and/or alternative non-limiting aspect of the present disclosure, the amount of agent included on, in and/or used in conjunction with metal alloy or medical device, when the agent is used, is about 0.01-100 μg per mm² (and all values and ranges wherein between) and/or at least about 0.00001 wt. % of device; however, other amounts can be used. The amount of two of more agents on, in and/or used in conjunction with medical device can be the same or different. The one or more agents can be coated on and/or impregnated in medical device by a variety of mechanisms such as, but not limited to, spraying (e.g., atomizing spray techniques, etc.), powder deposition, dip coating, flow coating, dip-spin coating, roll coating (direct and reverse), sonication, brushing, plasma deposition, depositing by vapor deposition, MEMS technology, and rotating mold deposition. The amount of two of more agents on, in and/or used in conjunction with medical device, when two one more agents are used, can be the same or different.

In accordance with another and/or alternative non-limiting aspect of the present disclosure, the one or more agents on and/or in the metal alloy or medical device, when used on the medical device, can be released in a controlled manner so the area in question to be treated is provided with the desired dosage of agent over a sustained period of time.

In accordance with another and/or alternative non-limiting aspect of the present disclosure, the one or more polymers used to at least partially control the release of one or more agents from the metal alloy or medical device can be porous or non-porous. The one or more agents can be inserted into and/or applied to one or more surface structures and/or micro-structures on the metal alloy or medical device, and/or be used to at least partially form one or more surface structures and/or micro-structures on the metal alloy or medical device.

In accordance with another and/or alternative non-limiting aspect of the present disclosure, the thickness of each polymer layer and/or layer of agent is generally at least about 0.01 μm and is generally less than about 150 μm (e.g., 0.01-149.9999 μm and all values and ranges therebetween). In one non-limiting embodiment, the thickness of a polymer layer and/or layer of agent is about 0.02-75 μm, more particularly about 0.05-50 μm, and even more particularly about 1-30 μm. As can be appreciated, other thicknesses can be used.

In accordance with another and/or alternative non-limiting aspect of the present disclosure, a variety of polymers can be coated on the metal alloy or medical device and/or be used to form at least a portion of the medical device. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification.

In accordance with another and/or alternative non-limiting aspect of the present disclosure, the medical device can optionally include a marker material that facilitates enabling the medical device to be properly positioned in a body passageway (e.g., blood vessel, heart valve, etc.). The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque).

In accordance with another and/or alternative non-limiting aspect of the present disclosure, the medical device or one or more regions of the medical device can optionally be constructed by use of one or more microelectromechanical manufacturing (MEMS) techniques (e.g., micro-machining, laser micro-machining, laser micro-machining, micro-molding, 3D printing, etc.); however, other or additional manufacturing techniques can be used.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology (e.g., 3D printing, etc.).

In accordance with another and/or alternative non-limiting aspect of the present disclosure, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie-like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm.

In still yet another and/or alternative non-limiting aspect of the present disclosure, the medical device can include and/or be used with a physical hindrance. The physical hindrance can include, but is not limited to, an adhesive, sheath, magnet, tape, wire, string, etc. The physical hindrance can be used to 1) physically retain one or more regions of the medical device in a particular form or profile, 2) physically retain the medical device on a particular deployment device, 3) protect one or more surface structures and/or micro-structures on the medical device, and/or 4) form a barrier between one or more surface regions, surface structures and/or micro-structures on the medical device and the fluids in a body passageway. The physical hindrance is optionally a biodegradable material; however, a biostable material can be used.

In still another and/or alternative aspect of the disclosure, the medical device can be an expandable device that can be expanded by use of some other device (e.g., balloon, etc.) and/or is self-expanding.

In accordance with another and/or alternative non-limiting aspect of the present disclosure, the medical device can optionally be fabricated from a material having no or substantially no shape-memory characteristics.

In accordance with another and/or alternative non-limiting aspect of the present disclosure, there is optionally provided a near net process for a frame, other metal component of the medical device, or a portion or all of another device. In one non-limiting embodiment of the disclosure, there is provided a method of powder pressing materials and optionally increasing the strength post-sintering by imparting additional cold work. In one non-limiting embodiment, the metal alloy powder is pressed into a green part and then sintered. Thereafter, the sintered part is optionally again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered, and re-pressed) meets the dimensional requirements of the final formed part. There is also provided an optional process of increasing the mechanical strength of a pressed metal part by repressing the post-sintered part to add additional cold work into the material, thereby increasing its mechanical strength. There is also provided an optional process of powder pressing to a near net or final part using metal powder. A prepress pressure of 1-300 Tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 Tsi (and all values and ranges therebetween) at a temperature of at least 20° C. (e.g., 20-100° C. and all values and ranges therebetween; 20-40° C., etc.). There is also provided a process of increasing the mechanical strength of a pressed metal part by repressing the post-sintered part to add additional cold work into the material, thereby increasing its mechanical strength. There is also provided a process of powder pressing to a near net or final part using metal powder. The ductility of the metal alloy measured as % reduction in area can increase and yield and ultimate tensile strength can increase.

In accordance with another and/or alternative non-limiting aspect of the present disclosure, there is optionally provided a press of near net or finished part composite (e.g., green part, etc.). The process of pressing metal alloy powder into near net of finished parts is well established; however, pressing a composite structure formed of metal powder and polymer for purposes of making complex part geometries and foam-like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, and then pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation (on the metal substrate) and the polymer residual has some desired biological affect (e.g., masking the metal from the body by encapsulation, promotion of cellular attachment and growth). The polymer and metal powders can be of varying sizes to create a multiplied of voids—some large, creating a pathway for cellular growth, and some small, creating a ruff surface to promote cellular attachment. As such, the use of a polymer in combination with metal powder and subsequent pressing and sintering can be used to form and customized shapes for medical device or the near net form of the medical device. Generally, the polymer constitutes about 0.1-70 vol. % (and all values and ranges therebetween) of the consolidated and pressed material prior to the sintering step, typically the polymer constitutes about 1-60 vol. % of the consolidated and pressed material prior to the sintering step, more typically the polymer constitutes about 2-50 vol. % of the consolidated and pressed material prior to the sintering step, and even more typically the polymer constitutes about 2-45 vol. % of the consolidated and pressed material prior to the sintering step. As such, if the polymer constitutes about 5 vol. % of the consolidated and pressed material prior to the sintering step, if after the sintering step at least 95% (e.g., 95-100% and all values and ranges therebetween) of the polymer is degraded and removed from the part or medical device, then the part could include up to about 5 vol. % cavities and/or passageways in the medical device. After the sintering process, at least 95 vol. % (95%-100% and all values and ranges therebetween) of the polymer is thermally degraded and/or removed from the sintered material.

In accordance with another and/or alternative non-limiting aspect of the present disclosure, the metal alloy that is used to at least partially form the medical device or other type of device is initially formed into a near net part, blank, a rod, a tube, etc., and then finished into final form by one or more finishing processes (e.g., centerless grinding, turning, electropolishing, drawing process, grinding, laser cutting, shaving, polishing, EDM cutting, micro-machining, laser micro-machining, micro-molding, machining, drilling (e.g., gun-drilling, etc.), 3D printing, cold wording, swaging, cleaning, buffing, smoothing, nitriding, annealing, plug drawing, etching (chemical etching, plasma etching, etc.), chemical modifications, chemical reactions, photo-etching, chemical coatings, etc.).

In accordance with another and/or alternative non-limiting aspect of the present disclosure, the metal alloy that partially or fully forms the near net part, blank, rod, tube, etc., can be formed by various techniques such as, but not limited to, 1) melting the metal alloy (e.g., vacuum arc melting, etc.) and then extruding and/or casting the metal alloy into a near net part, blank, rod, tube, etc., 2) melting the metal alloy, forming a metal strip and then rolling and welding the strip into a near net part, blank, rod, tube, etc., 3) consolidating (pressing, pressing and sintering, etc.) the metal powder of the metal alloy into a near net part, blank, rod, tube, etc., and/or 4) 3D print the metal alloy into a near net part, blank, rod, tube, etc. When the metal alloy is formed into a blank, the shape and size of the blank is non-limiting. When the metal alloy is formed into a rod or tube, the rod or tube generally has a length of about 48 inches or less (e.g., 0.1-48 inches and all values and ranges therebetween); however, longer lengths can be formed. In one non-limiting arrangement, the length of the rod or tube is about 8-20 inches. The average outer diameter of the rod or tube is generally less than about 2 inches (i.e., less than about 3.14 sq. in. cross-sectional area), more typically less than about 1 inch outer diameter, and even more typically no more than about 0.5 inch outer diameter; however, larger rod or tube diameter sizes can be formed. In one non-limiting configuration for a tube, the tube has an inner diameter of about 0.31 inch plus or minus about 0.002 inch and an outer diameter of about 0.5 inch plus or minus about 0.002 inch. The wall thickness of the tube is about 0.095 inch plus or minus about 0.002 inch. As can be appreciated, this is just one example of many different sized tubes that can be formed. In one non-limiting process, the near net medical device, blank, rod, tube, etc. can be formed from one or more ingots of metal or metal alloy.

In one non-limiting process, an arc melting process (e.g., vacuum arc melting process, etc.) can be used to form the near net medical device, blank, rod, tube, etc. In another non-limiting process, metal powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device, blank, rod, tube, etc. It can be appreciated that other or additional processes can be used to form the metal alloy. When a tube of metal alloy is to be formed, a close-fitting rod can be used during the extrusion process to form the tube; however, this is not required. In another and/or additional non-limiting process, the tube of the metal alloy can be formed from a strip or sheet of metal alloy. The strip or sheet of metal alloy can be formed into a tube by rolling the edges of the sheet or strip and then welding together the edges of the sheet or strip. The welding of the edges of the sheet or strip can be accomplished in several ways such as, but not limited to, a) holding the edges together and then e-beam welding the edges together in a vacuum, b) positioning a thin strip of metal alloy above and/or below the edges of the rolled strip or sheet to be welded, then welding the one or more strips along the rolled strip or sheet edges, and then grinding off the outer strip, or c) laser welding the edges of the rolled sheet or strip in a vacuum, oxygen reducing atmosphere, or inert atmosphere. In still another and/or additional non-limiting process, the near net medical device, blank, rod, tube, etc. of the metal alloy is formed by consolidating metal powder. In this process, fine particles of metal (e.g., Re, Hf, W, Mo, Ti, Cu, Ni, Cr, etc.) along with any additives are mixed to form a homogenous blend of particles. Typically, the average particle size of the metal powders is less than about 200 mesh (e.g., less than 74 microns). A larger average particle size can interfere with the proper mixing of the metal powders and/or adversely affect one or more physical properties of the near net medical device, blank, rod, tube, etc. formed from the metal powders. In one non-limiting embodiment, the average particle size of the metal powders is less than about 230 mesh (e.g., less than 63 microns). In another and/or alternative non-limiting embodiment, the average particle size of the metal powders is about 2-63 microns, and more particularly about 5-40 microns. As can be appreciated, smaller average particle sizes can be used. The purity of the metal powders should be selected so that the metal powders contain very low levels of carbon, oxygen and nitrogen. Typically, the carbon content of the metal powder used to form the metal alloy is less than about 100 ppm, the oxygen content is less than about 50 ppm, and the nitrogen content is less than about 20 ppm. Typically, metal powder used to form the metal alloy has a purity grade of at least 99.9 and more typically at least about 99.95. The blend of metal powder is then pressed together to form a solid solution of the metal alloy into a near net medical device, blank, rod, tube, etc. Typically the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen reducing atmosphere (e.g., hydrogen, argon and hydrogen mixture, etc.) and/or under a vacuum; however, this is not required. The average density of the near net medical device, blank, rod, tube, etc. that is achieved by pressing together the metal powders is about 80-95% (and all values and ranges therebetween) of the final average density of the near net medical device, blank, rod, tube, etc. or about 70-96% (and all values and ranges therebetween) the minimum theoretical density of the metal alloy. Pressing pressures of at least about 300 MPa are generally used. Generally, the pressing pressure is about 400-700 MPa; however, other pressures can be used. After the metal powders are pressed together, the pressed metal powders are sintered at a temperature of at least 1600° C. (e.g., 1600-3500° C. and all values and ranges therebetween) to partially or fully fuse the metal powders together to form the near net medical device, blank, rod, tube, etc. The sintering of the consolidated metal powder can be performed in an oxygen reducing atmosphere (e.g., helium, argon, hydrogen, argon and hydrogen mixture, etc.) and/or under a vacuum; however, this is not required. At the high sintering temperatures, a high hydrogen atmosphere will reduce both the amount of carbon and oxygen in the formed near net medical device, blank, rod, tube, etc. The sintered metal powder generally has an as-sintered average density of about 90-99% the minimum theoretical density of the metal alloy. Typically, the sintered metal alloy has a final average density of at least about 5 gm/cc, and typically at least about 8.3 gm/cc, and can be up to or greater than about 16 gm/cc; however, this is not required. The density of the formed near net medical device, blank, rod, tube, etc. will generally depend on the type of metal alloy used.

In accordance with another and/or alternative non-limiting aspect of the present disclosure, when a solid rod of the metal alloy is formed, the rod can optionally be formed into a tube prior to reducing the outer cross-sectional area or diameter of the rod. The rod can be formed into a tube by a variety of processes such as, but not limited to, cutting or drilling (e.g., gun-drilling, etc.) or by cutting (e.g., EDM, EDM sinker, wire EDM, etc.) or by 3D printing. The cavity or passageway formed in the rod typically is formed fully through the rod; however, this is not required.

In yet a further and/or alternative non-limiting aspect of the present disclosure, the near net medical device, blank, rod, tube, etc., can optionally be cleaned and/or polished after the near net medical device or other type of device, blank, rod, tube, etc., has been form; however, this is not required. Typically, the near net medical device or other type of device, blank, rod, tube, etc., is cleaned and/or polished prior to being further processed; however, this is not required.

In still yet a further and/or alternative non-limiting aspect of the present disclosure, the near net medical device, blank, rod, tube, etc. can be resized to the desired dimension of the medical device. In one non-limiting embodiment, the cross-sectional area or diameter of the near net medical device, blank, rod, tube, etc. is reduced to a final near net medical device, blank, rod, tube, etc. dimension in a single step or by a series of steps. The reduction of the outer cross-sectional area or diameter of the near net medical device, blank, rod, tube, etc. may be obtained by centerless grinding, turning, electropolishing, drawing process, grinding, laser cutting, shaving, polishing, EDM cutting, etc. The outer cross-sectional area or diameter size of the near net medical device, blank, rod, tube, etc. can be reduced by the use of one or more drawing processes; however, this is not required. During the drawing process, care should be taken to not form micro-cracks in the near net medical device, blank, rod, tube, etc. during the reduction of the near net medical device, blank, rod, tube, etc. outer cross-sectional area or diameter. Generally, the near net medical device, blank, rod, tube, etc. should not be reduced in cross-sectional area by more about 25% each time the near net medical device, blank, rod, tube, etc. is drawn through a reducing mechanism (e.g., a die, etc.). In one non-limiting process step, the near net medical device, blank, rod, tube, etc. is reduced in cross-sectional area by about 0.1-20% each time the near net medical device, blank, rod, tube, etc. is drawn through a reducing mechanism. In another and/or alternative non-limiting process step, the near net medical device, blank, rod, tube, etc. is reduced in cross-sectional area by about 1-15% each time the near net medical device, blank, rod, tube, etc. is drawn through a reducing mechanism. In still another and/or alternative non-limiting process step, the near net medical device, blank, rod, tube, etc. is reduced in cross-sectional area by about 2-15% each time the near net medical device, blank, rod, tube, etc. is drawn through reducing mechanism. In yet another one non-limiting process step, the near net medical device, blank, rod, tube, etc. is reduced in cross-sectional area by about 5-10% each time the near net medical device, blank, rod, tube, etc. is drawn through reducing mechanism. In another and/or alternative non-limiting embodiment of the disclosure, the near net medical device, blank, rod, tube, etc. of metal alloy is drawn through a die to reduce the cross-sectional area of the near net medical device, blank, rod, tube, etc. Generally, before drawing the near net medical device, blank, rod, tube, etc. through a die, one end of the near net medical device, blank, rod, tube, etc. is narrowed down (nosed) so as to allow it to be fed through the die; however, this is not required. The tube drawing process is typically a cold drawing process or a plug drawing process through a die. When a cold drawing or mandrel drawing process is used, a lubricant (e.g., molybdenum paste, grease, etc.) is typically coated on the outer surface of the near net medical device, blank, rod, tube, etc. and the near net medical device, blank, rod, tube, etc. is then drawn though the die. Typically, little or no heat is used during the cold drawing process. After the near net medical device, blank, rod, tube, etc. has been drawn through the die, the outer surface of the near net medical device, blank, rod, tube, etc. is typically cleaned with a solvent to remove the lubricant so as to limit the amount of impurities that are incorporated in the metal alloy; however, this is not required. This cold drawing process can be repeated several times until the desired outer cross-sectional area or diameter, inner cross-sectional area or diameter and/or wall thickness of the near net medical device, blank, rod, tube, etc. is achieved. A plug drawing process can also or alternatively be used to size the near net medical device, blank, rod, tube, etc. The plug drawing process typically does not use a lubricant during the drawing process. The plug drawing process typically includes a heating step to heat the near net medical device, blank, rod, tube, etc. prior and/or during the drawing of the near net medical device, blank, rod, tube, etc. through the die. The elimination of the use of a lubricant can reduce the incidence of impurities being introduced into the metal alloy during the drawing process. During the plug drawing process, the near net medical device, blank, rod, tube, etc. can be protected from oxygen by use of a vacuum environment, a non-oxygen environment (e.g., hydrogen, argon and hydrogen mixture, nitrogen, nitrogen and hydrogen, etc.) or an inert environment. One non-limiting protective environment includes argon, hydrogen or argon and hydrogen; however, other or additional inert gasses can be used. As indicated above, the near net medical device, blank, rod, tube, etc. is typically cleaned after each drawing process to remove impurities and/or other undesired materials from the surface of the near net medical device, blank, rod, tube, etc.; however, this is not required. Typically, the near net medical device, blank, rod, tube, etc. should be shielded from oxygen and nitrogen when the temperature of the near net medical device, blank, rod, tube, etc. is increased to above 500° C., and typically above 450° C., and more typically above 400° C.; however, this is not required. When the near net medical device, blank, rod, tube, etc. is heated to temperatures above about 400-500° C., the near net medical device, blank, rod, tube, etc. tends to begin forming nitrides and/or oxides in the presence of nitrogen and oxygen. In these higher temperature environments, a hydrogen environment, an argon and hydrogen environment, etc. is generally used. When the near net medical device, blank, rod, tube, etc. is drawn at temperatures below 400-500° C., the near net medical device, blank, rod, tube, etc. can be exposed to air with little or no adverse effects; however, an inert or slightly reducing environment is generally more desirable.

In another and/or alternative non-limiting aspect of the present disclosure, a rod of metal alloy is partially or fully reduced to the desired outer diameter prior to forming a tube from the rod. Once the solid metal rod is formed, the outer diameter of the metal rod is reduced by cold working the rod using or more techniques such as, but not limited to, use of a mandrel, use of one or more rollers, use of an extrusion process, use of a die, squeezing processes, cold rolling process, a rotary swaging process, cold forging process, sizing processes, drawing processes, etc. The rod can be processed one or more times using one or more cold working techniques prior to achieving the desired outer diameter of the rod. Generally, the rod is not reduced in cross-sectional area by more about 25% each time the rod is subjected to a reducing mechanism. During the reducing process, the solid rod can be annealed one or more times to facilitate in the reduction of the outer diameter of the rod. The rod may or may not be annealed each time the rod is subjected to a reducing mechanism. Once the solid rod has obtained it desired outer diameter, the solid rod is typically not further annealed so as to maintain the hardness properties and yield strength properties that were obtained by the cold working of the rod. The above discussed parameters for reducing the outer diameter of the solid rod to the desired outer diameter can be used. Once the solid rod has the desired outer diameter, the solid rod is formed into a tube by gun-drilling the rod or EDM cutting of the rod. Further processing of the tube can occur to finalize the wall thickness of the tube and uniformity of the wall thickness of the tube. One non-limiting process that can be used is a wire EDM machining process. Once the tube is formed, the tube can be subject to one or more treatment processes such as cleaning, polishing, sterilizing, nitrided, etc. for final processing of the metal tube. The metal tube can be further processed by cutting the metal tube into a portion of or the complete final form (e.g., final medical device-stent, TAVR, screw, etc.). It has been found that metal tubes that are first formed by reducing the outer diameter of the rod and then hollowing out the rod to form a tube have a final yield strength of at least 30% greater than tubes that are first formed and then the outer diameter of the tube is reduced. For example, metal alloy tubes that have yield strengths of 130 Ksi when formed from a tube that has been reduced in outer diameter (and not annealed after achieving its final outer diameter) can have yield strengths of at least 200 Ksi when formed by first reducing a solid rod by a cold working process and then hollowing out the solid rod to form a tube after the rod has been reduced to the desired outer diameter.

In accordance with another and/or alternative aspect of the present disclosure, when metal powder is used to 3D print a frame for a prosthetic heart valve, component of a frame for a prosthetic heart valve, blank, rod, tube, etc., the average particle size of the metal powder is optionally 2-62 microns, and more particularly about 5-49.9 microns, the average density of the metal powders is greater than 5 g/cm³, and the metal powder is generally spherical-shaped, and the Hall flow (s/50 g) is less than 30 seconds (e.g., 2-29.99 seconds and all values and ranges therebetween).

In still a another and/or alternative non-limiting aspect of the present disclosure, the near net medical device, blank, rod, tube, etc. during the drawing process can be nitrided; however, this is not required. The nitride layer on the near net medical device, blank, rod, tube, etc. can function as a lubricating surface during the drawing process to facilitate in the drawing of the near net medical device, blank, rod, tube, etc. The near net medical device, blank, rod, tube, etc. is generally nitrided in the presence of nitrogen or a nitrogen mixture. In one non-limiting embodiment of the disclosure, the surface of the near net medical device, blank, rod, tube, etc. is nitrided prior to at least one drawing step for the near net medical device, blank, rod, tube, etc. In one non-limiting aspect of this embodiment, the surface of the near net medical device, blank, rod, tube, etc. is nitrided prior to a plurality of drawing steps. In another non-limiting aspect of this disclosure, after the near net medical device, blank, rod, tube, etc. has been annealed, the near net medical device, blank, rod, tube, etc. is nitrided prior to being drawn. In another and/or alternative non-limiting embodiment, the near net medical device, blank, rod, tube, etc. is cleaned to remove nitride compounds on the surface of the near net medical device, blank, rod, tube, etc. prior to annealing the rod to tube. The nitride compounds can be removed by a variety of steps such as, but not limited to, grit blasting, polishing, etc. After the near net medical device, blank, rod, tube, etc. has been annealed, the near net medical device, blank, rod, tube, etc. can be again nitri ded prior to one or more drawing steps; however, this is not required. As can be appreciated, the complete outer surface of the near net medical device, blank, rod, tube, etc. can be nitrided or a portion of the outer surface of the near net medical device, blank, rod, tube, etc. can be nitrided. Nitriding only selected portions of the outer surface of the near net medical device, blank, rod, tube, etc. can be used to obtain different surface characteristics of the near net medical device, blank, rod, tube, etc.; however, this is not required.

In still yet a another and/or alternative non-limiting aspect of the present disclosure, the near net medical device, blank, rod, tube, etc. is annealed after one or more drawing processes. The metal alloy blank, rod, tube, etc. can be annealed after each drawing process or after a plurality of drawing processes. The metal alloy blank, rod, tube, etc. is typically annealed prior to about a 60% cross-sectional area size reduction of the metal alloy blank, rod, tube, etc. In other words, the near net medical device, blank, rod, tube, etc. should not be reduced in cross-sectional area by more than 60% before being annealed (e.g., 0.1-60% reduction and all values and ranges therebetween). A too-large reduction in the cross-sectional area of the metal alloy blank, rod, tube, etc. during the drawing process prior to the near net medical device, blank, rod, tube, etc. being annealed can result in micro-cracking of the near net medical device, blank, rod, tube, etc. In one non-limiting processing step, the metal alloy blank, rod, tube, etc. is annealed prior to about a 50% cross-sectional area size reduction of the metal alloy blank, rod, tube, etc. In another and/or alternative non-limiting processing step, the metal alloy blank, rod, tube, etc. is annealed prior to about a 45% cross-sectional area size reduction of the metal alloy blank, rod, tube, etc. In still another and/or alternative non-limiting processing step, the metal alloy blank, rod, tube, etc. is annealed prior to about a 1-45% cross-sectional area size reduction of the metal alloy blank, rod, tube, etc. In yet another and/or alternative non-limiting processing step, the metal alloy blank, rod, tube, etc. is annealed prior to about a 5-30% cross-sectional area size reduction of the metal alloy blank, rod, tube, etc. In still yet another and/or alternative non-limiting processing step, the metal alloy blank, rod, tube, etc. is annealed prior to about a 5-15% cross-sectional area size reduction of the metal alloy blank, rod, tube, etc.

In another and/or alternative non-limiting aspect of the disclosure, when the near net medical device, blank, rod, tube, etc. is annealed, the near net medical device, blank, rod, tube, etc. is typically heated to a temperature of about 500-1700° C. (and all values and ranges therebetween) for a period of about 1-200 minutes (and all values and ranges therebetween); however, other temperatures and/or times can be used. In one non-limiting processing step, the near net medical device, blank, rod, tube, etc. is annealed at a temperature of about 1000-1600° C. for about 2-100 minutes. In another non-limiting processing step, the near net medical device, blank, rod, tube, etc. is annealed at a temperature of about 1100-1500° C. for about 5-30 minutes. The annealing process typically occurs in an inert environment or an oxygen-reducing environment so as to limit the amount of impurities that may embed themselves in the metal alloy during the annealing process. One non-limiting oxygen-reducing environment that can be used during the annealing process is a hydrogen environment; however, it can be appreciated that a vacuum environment can be used or one or more other or additional gasses can be used to create the oxygen-reducing environment. At the annealing temperatures, a hydrogen-containing atmosphere can further reduce the amount of oxygen in the near net medical device, blank, rod, tube, etc. The chamber in which the near net medical device, blank, rod, tube, etc. is annealed should be substantially free of impurities (e.g., carbon, oxygen, and/or nitrogen) so as to limit the amount of impurities that can embed themselves in the near net medical device, blank, rod, tube, etc. during the annealing process.

In another and/or alternative non-limiting aspect of the present disclosure, the parameters for annealing can be changed as the near net medical device, blank, rod, tube, etc. as the cross-sectional area or diameter; and/or wall thickness of the near net medical device, blank, rod, tube, etc. are changed. It has been found that good grain size characteristics of the near net medical device, blank, rod, tube, etc. can be achieved when the annealing parameters are varied as the parameters of the near net medical device, blank, rod, tube, etc. change. For example, as the wall thickness is reduced, the annealing temperature is correspondingly reduced; however, the times for annealing can be increased. As can be appreciated, the annealing temperatures of the near net medical device, blank, rod, tube, etc. can be decreased as the wall thickness decreases, but the annealing times can remain the same or also be reduced as the wall thickness reduces. After each annealing process, the grain size of the metal in the near net medical device, blank, rod, tube, etc. should be no greater than 4 ASTM. Generally, the grain size range is about 4-20 ASTM (and all values and ranges therebetween). It is believed that as the annealing temperature is reduced as the wall thickness reduces, small grain sizes can be obtained. The grain size of the metal in the near net medical device, blank, rod, tube, etc. should be as uniform as possible. In addition, the sigma phase of the metal in the near net medical device, blank, rod, tube, etc. should be as reduced as much as possible. The sigma phase is a spherical, elliptical or tetragonal crystalline shape in the metal alloy.

In another and/or alternative non-limiting aspect of the present disclosure, after the final drawing of the near net medical device, blank, rod, tube, etc., a final annealing of the near net medical device, blank, rod, tube, etc. can be done for final strengthening of the near net medical device, blank, rod, tube, etc.; however, this is not required. This final annealing process, when used, generally occurs at a temperature of about 500-1600° C. (and all values and ranges therebetween) for at least about 1 minutes; however, other temperatures and/or time periods can be used.

In another and/or alternative non-limiting aspect of the present disclosure, the near net medical device or other type of device, blank, rod, tube, etc. general if not reduced in cross-sectional area by more about 25% (e.g., 0.1-25% and all values and ranges therebetween) each time the near net medical device or other type of device, blank, rod, tube, etc. is drawn down in size.

In yet a another and/or alternative non-limiting aspect of the present disclosure, the near net medical device, blank, rod, tube, etc. is cooled after being annealed; however, this is not required. Generally, the near net medical device, blank, rod, tube, etc. is cooled at a fairly quick rate after being annealed so as to inhibit or prevent the formation of a sigma phase in the metal alloy; however, this is not required. Generally, the near net medical device, blank, rod, tube, etc. is cooled at a rate of at least about 50° C. per minute (e.g., 50-500° C. per minute and all values and ranges therebetween) after being annealed, typically at least about 75° C. per minute after being annealed, more typically at least about 100° C. per minute after being annealed, even more typically about 100-400° C. per minute after being annealed, still even more typically about 150-350° C. per minute after being annealed, and yet still more typically about 200-300° C. per minute after being annealed, and still yet even more typically about 250-280° C. per minute after being annealed; however, this is not required.

In accordance with another and/or alternative non-limiting aspect of the present disclosure, the near net medical device or other type of device, blank, rod, tube, etc. can be cleaned prior to and/or after being annealed.

In another and/or alternative non-limiting aspect of the present disclosure, the near net medical device, blank, rod, tube, etc. can be cleaned prior to and/or after being annealed. The cleaning process is designed to remove impurities, lubricants (e.g., nitride compounds, molybdenum paste, grease, etc.) and/or other materials from the surfaces of the near net medical device, blank, rod, tube, etc. Impurities that are on one or more surfaces of the near net medical device, blank, rod, tube, etc. can become permanently embedded into the near net medical device, blank, rod, tube, etc. during the annealing processes. These imbedded impurities can adversely affect the physical properties of the metal alloy as the near net medical device, blank, rod, tube, etc. is formed into a medical device, and/or can adversely affect the operation and/or life of the medical device. In one non-limiting embodiment of the disclosure, the cleaning process includes a delubrication or degreasing process which is typically followed by pickling process; however, this is not required. The delubrication or degreasing process followed by pickling process is typically used when a lubricant has been used on the near net medical device, blank, rod, tube, etc. during a drawing process. Lubricants commonly include carbon compounds, nitride compounds, molybdenum paste, and other types of compounds that can adversely affect the metal alloy if such compounds and/or elements in such compounds become associated and/or embedded with the metal alloy during an annealing process. The delubrication or degreasing process can be accomplished by a variety of techniques such as, but not limited to, 1) using a solvent (e.g., acetone, methyl alcohol, etc.) and wiping the metal alloy with a Kimwipe or other appropriate towel, 2) by at least partially dipping or immersing the metal alloy in a solvent and then ultrasonically cleaning the metal alloy, 3) sand blasting the metal alloy, and/or 4) chemical etching the metal alloy. As can be appreciated, the metal alloy can be delubricated or degreased in other or additional ways. After the near net medical device, blank, rod, tube, etc. has been delubricated or degreased, the near net medical device, blank, rod, tube, etc. can be further cleaned by use of a pickling process; however, this is not required. The pickling process (when used) includes the use of one or more acids to remove impurities from the surface of the near net medical device, blank, rod, tube, etc. Non-limiting examples of acids that can be used as the pickling solution include, but are not limited to, nitric acid, acetic acid, sulfuric acid, hydrochloric acid, and/or hydrofluoric acid. These acids are typically analytical reagent (ACS) grade acids. The acid solution and acid concentration are selected to remove oxides and other impurities on the near net medical device, blank, rod, tube, etc. surface without damaging or over-etching the surface of the near net medical device, blank, rod, tube, etc.

In accordance with another and/or alternative non-limiting aspect of the present disclosure, the near net medical device or other type of device, blank, rod, tube, etc., after a) being formed to the desired green shape, b) after being formed to have the desired outer cross-sectional area or diameter, and/or c) after being formed to have the desired inner cross-sectional area or diameter and/or wall thickness, can then be cut and/or etched to at least partially form the desired configuration of the medical device (e.g., stent, frame for TAV valve, orthopedic device, vascular device, etc.). The near net medical device or other type of device, blank, rod, tube, etc. can be cut or otherwise formed by one or more processes (e.g., centerless grinding, turning, electropolishing, drawing process, grinding, laser cutting, shaving, polishing, EDM cutting, etching, micro-machining, laser micro-machining, micro-molding, machining, etc.). As can be appreciated, a portion or all of the medical device or other type of device can be formed by 3D printing.

In still another and/or alternative non-limiting aspect of the present disclosure, the metal alloy, after being formed to the desired shape, the outer cross-sectional area or diameter, inner cross-sectional area or diameter and/or wall thickness, can be cut and/or etched to at least partially form the desired configuration of the medical device (e.g., stent, TAVR valve frame, pedicle screw, PFO device, spinal implant, vascular implant, rod, guide wire, sheath frame, stent hypotube, etc.). The near net medical device, blank, rod, tube, etc. can be cut or otherwise formed by one or more processes (e.g., centerless grinding, turning, electropolishing, drawing process, grinding, laser cutting, shaving, polishing, EDM cutting, etching, micro-machining, laser micro-machining, micro-molding, machining, etc.). In one non limiting embodiment of the disclosure, the metal alloy near net medical device, blank, rod, tube, etc. is at least partially cut by a laser.

In accordance with another and/or alternative non-limiting aspect of the present disclosure, the metal alloy can be coated with an enhancement coating to improve one or more properties of the metal alloy (e.g., change exterior color of metal alloy, increase hardness of coated surface, increase toughness of coated surface, reduced friction to coated surface, improve impact wear of coated surface, improve resistance to corrosion and oxidation, form a non-stick coated surface, improve biocompatibility of metal alloy having the coated surface, reduce toxicity of metal alloy having the coated surface, etc.). In some vascular applications, the use of the enhancement coating one or more portions of the metal alloy that forms all or a portion of the medical device (e.g., stent, frame of a heart valve, etc.) and/or on a portion of the medical device that does not includes the metal alloy (e.g., leaflet of a heart valve, outer skirt of a heart valve, inner skirt of a heart valve, etc.), the enhancement coating, after the medical device has been implanted into the vascular system, can inhibit or prevent calcium deposits on one or more portions of the medical device (e.g., inhibit or prevent calcium deposits on frame, leaflets, skirt, etc.). Such reduction in calcium deposits on the medical device can a) extend the life of the medical device, and/or b) inhibit or prevent interference with the proper operation of the medical device. The enhancement coating can be applied prior to or after the optional further processing of the medical device or other type of device into its final form. For example, if a rod or tube of metal alloy is to be further cut, etched, nitrided, swaged, heat treated, drawing down, bent, etc. to form all or a portion of the medical device or other type of device, the enhancement coating can be applied to the tube or rod after such cutting, etching, nitriding, swaging, heat treating, drawing down, bending, etc. In one non-limiting embodiment, a portion or all of the medical device or other type of device is formed of a metal alloy, and wherein a portion or all of the outer surface of the metal alloy is coated with an enhancement coating (e.g., chromium nitride (CrN), diamond-like carbon (DLC), titanium nitride (TiN), zirconium nitride (ZrN), zirconium oxide (ZrO₂), zirconium-nitrogen-carbon (ZrNC), zirconium OxyCarbide (ZrOC), and wherein the outer surface of the metal alloy optionally includes an adhesion layer, which adhesion layer is optionally a metallic layer that includes titanium or zirconium. In another non-limiting embodiment, the metal alloy is coated with an enhancement coating, and wherein the outer surface of the metal alloy optionally includes an adhesion layer, which adhesion layer is optionally a metallic layer that includes titanium or zirconium.

Another and/or alternative non-limiting aspect of the present disclosure is the provision of a of a medical device or other type of device that is coated with an enhancement layer (e.g., metal oxynitride layer) that facilitates in the formation of a) nitric oxide (NO) production, b) stimulation of endothelial cells, and/or c) a modulation of endothelial cells. Non-limiting enhancement coatings that can be applied to a portion of all of the outer surface of the medical device or other type of device includes chromium nitride (CrN), diamond-like carbon (DLC), titanium nitride (TiN), titanium oxynitride or titanium nitride oxide (TiNOx), zirconium nitride (ZrN), zirconium oxide (ZrO2), zirconium oxynitride (ZrNxOy) [e.g., cubic ZrN:O, cubic ZrO2:N, tetragonal ZrO2:N, and monoclinic ZrO2:N phase coatings], oxyzirconium-nitrogen-carbon (ZrNC), zirconium OxyCarbide (ZrOC), and combinations of such coatings. In one non-limiting embodiment, the one or more enhancement coatings are applied to a portion of all of the outer surface of the metal alloy that includes at least 15 atw. % hafnium can be a vacuum process using an energy source to vaporize material and deposit a thin layer of enhancement coating material. Such vacuum coating process includes a physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process. In one non-limiting embodiment, the coating process is one or more of a PVD, CVD, ALD and PE-CVD, and wherein the coating process occurs at a temperature of 200-400° C. (and all values and ranges therebetween) for at least 10 minutes (e.g., 10-400 minutes and all values and ranges therebetween). In another non-limiting embodiment, the coating process is one or more of a PVD, CVD, ALD and PE-CVD, and wherein the coating process occurs at a temperature of 220-300° C. for 60-120 minutes. The materials of the one or more enhancement coatings can be combine with one or more metals in the metal alloy, and/or combined with nitrogen, oxygen, carbon, or other elements that are in the metal alloy and/or present in the atmosphere about the metal alloy to a form an enhancement coating on the outer surface of the metal alloy that can have enhanced properties (e.g., enhancement coating is harder than case-hardened steel, enhancement coating is more scratch-resistant than hardened chrome, enhancement coating having high corrosion resistance, etc.). In another non-limiting embodiment, the one or more enhancement coatings can form various coating colors on the outer surface of the metal alloy (e.g., gold, copper, brass, black, rose gold, chrome, blue, silver, yellow, green, etc.). In another non-limiting embodiment, the thickness of the enhancement coating is greater than 1 nanometer (e.g., 2 nanometers to 100 microns and all values and ranges therebetween), and typically 0.1-25 microns, and more typically 1-10 microns. In another non-limiting embodiment, the hardness of the enhancement coating is at 5 GPa (ASTM C1327-15 or ASTM C1624-05), typically 5-50 GPa (and all values and ranges therebetween), more typically 10-25 GPa, and still more typically 14-24 GPa. In another non-limiting embodiment, the coefficient of friction (COF) of the enhancement coating is 0.04-0.2 (and all values and ranges therebetween), and typically 0.6-0.15. In another non-limiting embodiment, the wear rate of the enhancement coating is 0.5×10⁻⁷ mm³/N-m to 3×10⁻⁷ mm³/N-m (and all values and ranges therebetween), and typically 1.2×10⁻⁷ mm³/N-m to 2×10⁻⁷ mm³/N-m. In another non-limiting embodiment, silicon-based precursors (e.g., trimethysilane, tetramethylsilane, hexachlorodisilane, silane, dichlorosilane, trichlorosilane, silicon tetrachloride, tris(dimethylamino) silane, bis(tert-butylamino) silane, trisilylamine, allyltrimethoxysilane, (3-aminopropyl)triethoxysilane, butyltrichlorosilane, n-sec-butyl (trimethylsilyl) amine, chloropentamethyldisilane, 1,2-dichlorotetramethyldisilane, [3-(diethylamino)propyl]trimethoxysilane, 1,3-diethyl-1,1,3,3-tetramethyldisilazane, dimethoxydimethylsilane, dodecamethylcyclohexasilane, hexamethyldisilane, isobutyl (trimethoxy) silane, methyltrichlorosilane, 2,4,6,8,10-pentamethylcyclopentasiloxane, pentamethyldisilane, n-propyltriethoxysilane, silicon tetrabromide, silicon tetrabromide, etc.) can be used to facilitate in the application of the enhancement coating to one or more portions or all of the outer surface of the metal alloy. In one non-limiting embodiment, the enhancement coating optionally includes no more than 0.1 wt. % nickel, no more than 0.1 wt. % chromium, and/or no more than 0.1 wt. % cobalt. In another non-limiting embodiment, the outer surface of the medical device or other type of device optionally includes no more than 0.1 wt. % nickel, no more than 0.1 wt. % chromium, and/or no more than 0.1 wt. % cobalt. The adhesion layer optionally includes no more than 0.1 wt. % nickel, no more than 0.1 wt. % chromium, and/or no more than 0.1 wt. % cobalt. The metal alloy that forms a portion or all of the medical device or other type of device optionally includes no more than 0.1 wt. % nickel, no more than 0.1 wt. % chromium, and/or no more than 0.1 wt. % cobalt.

Another and/or alternative non-limiting object of the present disclosure is the provision of a medical device or other type of device wherein the enhancement coating is partially or fully applied to a metallic adhesion layer. In one non-limiting embodiment, the metallic adhesion layer optionally includes titanium metal or zirconium metal. In another on-limiting embodiment, the metallic adhesion layer optionally has a thickness of 1 to 500 nanometers (and all values and ranges therebetween). The enhancement coating and/or the metallic adhesion layer can be applied by use of a vacuum coating process (e.g., physical vapor deposition (PVD) process (e.g., sputter deposition, cathodic arc deposition or electron beam heating, etc.), chemical vapor deposition (CVD) process, atomic layer deposition (ALD) process, or a plasma-enhanced chemical vapor deposition (PE-CVD) process), plating process, etc.

In accordance with another and/or alternative non-limiting aspect of the present disclosure, the metal alloy is coated with an enhancement coating to improve one or more properties of the metal alloy wherein the enhancement coating composition includes a chromium nitride (CrN) coating. A portion or all of the outer surface of the metal alloy can include the chromium nitride (CrN) coating. The enhancement coating can be used to improve hardness, improve toughness, reduced friction, resistant impact wear, improve resistance to corrosion and oxidation, and/or form a reduced stick surface when in contact with many different materials. In accordance with one non-limiting embodiment, the metal alloy is coated with an enhancement coating that generally includes 40-85 wt. % Cr (and all values and ranges therebetween), 15-60 wt. % N (and all values and ranges therebetween), 0-10 wt. % Re (and all values and ranges therebetween), 0-10 wt. % Si (and all values and ranges therebetween), 0-2 wt. % O (and all values and ranges therebetween), and 0-2 wt. % C (and all values and ranges therebetween). In one non-limiting coating process, all or a portion of the outer surface of the metal alloy is initially coated with Cr metal. The Cr metal coating can be applied by PVD, CVD, ALD and PE-CVD in an inert environment. The coating thickness of Cr metal is 0.5-15 microns. Thereafter, the Cr metal coating is exposed to nitrogen gas and/or a nitrogen containing gas compound to cause the nitrogen to react with the Cr metal coating to form a layer of CrN on the outer surface of the Cr metal coating and/or the outer surface of the metal alloy. In another non-limiting embodiment, the enhancement coating composition generally includes 65-80 wt. % Cr, 15-30 wt. % N, 0-8 wt. % Re, 0-1 wt. % Si, 0-1 wt. % O, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes a diamond-Like Carbon (DLC) coating. A portion or all of the outer surface of the metal alloy can include the diamond-Like Carbon (DLC) coating. The enhancement coating can be used to improve hardness, improve toughness, reduced friction, resistant impact wear, improve resistance to corrosion and oxidation, improve biocompatibility, and/or form a reduced stick surface when in contact with many different materials. In one non-limiting embodiment, all or a portion of the outer surface of the metal alloy is coated with the enhancement coating composition that generally includes 60-99.99 wt. % C (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % O (and all values and ranges therebetween). The carbon coating can be applied by PVD, CVD, ALD and PE-CVD in an inert environment. The carbon layer can be applied by using methane and/or acetylene gas; however, other or additional carbon sources can be used. The coating thickness of the carbon is 0.5-15 microns. In another non-limiting embodiment, all or a portion of the outer surface of the metal alloy is coated with the enhancement coating composition that generally includes 90-99.99 wt. % C, 0-1 wt. % N, 0-8 wt. % Re, 0-1 wt. % Si, and 0-1 wt. % O.

In accordance with another and/or alternative non-limiting aspect of the present disclosure, the metal alloy is coated with an enhancement coating to improve one or more properties of the metal alloy wherein the enhancement coating composition generally includes a titanium nitride (TiN) coating. A portion or all of the outer surface of the metal alloy can include the titanium nitride (TiN) coating. The enhancement coating can be used to improve hardness, improve toughness, improve resistance to corrosion and oxidation, reduced friction, and/or form a reduced stick surface when in contact with many different materials. In one non-limiting embodiment, all or a portion of the outer surface of the metal alloy is initially coated with Ti metal, which Ti metal is a component of the enhancement coating. The Ti metal coating can be applied by PVD, CVD, ALD and PE-CVD in an inert environment. The coating thickness of Ti metal is 0.5-15 microns. Thereafter, the Ti metal coating is exposed to nitrogen gas and/or a nitrogen containing gas compound to cause the nitrogen to react with the Ti metal coating to form a layer of TiN on the outer surface of the Ti metal coating and/or the outer surface of the metal alloy. In another non-limiting embodiment, the enhancement coating composition generally includes 20-85 wt. % Ti (and all values and ranges therebetween), 5-30 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 70-80 wt. % Ti, 20-25 wt. % N, 0-8 wt. % Re, 0-1 wt. % Si, 0-1 wt. % O, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes a zirconium nitride (ZrN) coating. A portion or all of the outer surface of the metal alloy can include the zirconium nitride (ZrN) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound to cause the nitrogen to react with the Zn metal coating to form a layer of ZrN on the outer surface of the Zr metal coating and/or the outer surface of the metal alloy. The ZrN coating has been found to produce a gold-colored enhancement coating color. In another non-limiting embodiment, the enhancement coating composition generally includes 35-90 wt. % Zr (and all values and ranges therebetween), 5-25 wt. % N (and all values and ranges therebetween), 0-10 wt. % Re (and all values and ranges therebetween), 0-20 wt. % Si (and all values and ranges therebetween), 0-2 wt. % O (and all values and ranges therebetween), and 0-2 wt. % C (and all values and ranges therebetween). In another non-limiting embodiment, the enhancement coating composition generally includes 80-90 wt. % Zr, 10-20 wt. % N, 0-8 wt. % Re, 0-1 wt. % Si, 0-1 wt. % O, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes a zirconium oxide (ZrO₂) coating. A portion or all of the outer surface of the metal alloy can include the zirconium oxide (ZrO₂) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to oxygen gas and/or oxygen containing gas compound to cause the oxygen to react with the Zn metal coating to form a layer of zirconium oxide (ZrO₂) on the outer surface of the Zr metal coating and/or the outer surface of the metal alloy. The zirconium oxide (ZrO₂) coating has been found to produce a blue colored enhancement coating color. In another non-limiting embodiment, the enhancement coating composition generally includes 35-90 wt. % Zr (and all values and ranges therebetween), 10-35 wt. % O (and all values and ranges therebetween), 0-2 wt. % N (and all values and ranges therebetween), 0-10 wt. % Re (and all values and ranges therebetween), 0-20 wt. % Si (and all values and ranges therebetween), and 0-2 wt. % C (and all values and ranges therebetween). In another non-limiting embodiment, the enhancement coating composition generally includes 70-80 wt. % Zr, 20-30 wt. %, 0-1 wt. % N, 0-8 wt. % Re, 0-1 wt. % Si, and 0-1 wt. % C.

In accordance with another and/or alternative non-limiting aspect of the present disclosure, the metal alloy is coated with an enhancement coating to improve one or more properties of the metal alloy wherein the enhancement coating composition generally includes both a zirconium oxide (ZrO2) coating and a zirconium nitride coating (ZrN). A portion or all of the outer surface of the metal alloy can include the zirconium oxide (ZrO2) coating and the zirconium nitride coating (ZrN). The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to a) both oxygen gas and/or oxygen containing gas compound and also to nitrogen gas and/or nitrogen containing gas compound, b) nitrogen gas and/or nitrogen containing gas compound and then to oxygen gas and/or oxygen containing gas compound, or c) oxygen gas and/or oxygen gas containing compound and then to nitrogen gas and/or nitrogen gas containing compound. The coating composition of the zirconium oxide (ZrO2) coating and the zirconium nitride coating (ZrN) are similar or the same as discussed above.

In accordance with another and/or alternative non-limiting aspect of the present disclosure, the metal alloy is coated with an enhancement coating to improve one or more properties of the metal alloy wherein the enhancement coating composition generally includes a zirconium oxycarbide (ZrOC) coating. A portion or all of the outer surface of the metal alloy can include the zirconium oxycarbide (ZrOC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to a) both to oxygen gas and/or an oxygen containing gas compound and to carbon and/or a carbon containing gas compound (e.g., methane and/or acetylene gas), b) carbon and/or a carbon containing gas compound and then to oxygen gas and/or an oxygen containing gas compound, or c) oxygen gas and/or oxygen containing gas compound and then to carbon and/or carbon containing gas compound. In another non-limiting embodiment, the enhancement coating composition generally includes 40-95 wt. % Zr (and all values and ranges therebetween), 5-25 wt. % O (and all values and ranges therebetween), and 10-40 wt. % C (and all values and ranges therebetween), 0-2 wt. % N (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-65 wt. % Zr, 5-25 wt. % O, and 25-40 wt. % C, 0-1 wt. % N, 0-8 wt. % Re, and 0-1 wt. % Si.

In accordance with another and/or alternative non-limiting aspect of the present disclosure, the metal alloy is coated with an enhancement coating to improve one or more properties of the metal alloy wherein the enhancement coating composition generally includes a zirconium-nitrogen-carbon (ZrNC) coating. A portion or all of the outer surface of the metal alloy can include the zirconium-nitrogen-carbon (ZrNC) coating. The enhancement coating can be used to improve hardness, improve toughness, improve resistance to corrosion and oxidation, reduced friction, and/or form a reduced stick surface when in contact with many different materials. In one non-limiting embodiment all or a portion of the outer surface of the metal alloy is initially coated with Zr metal, which Zr metal is a component of the enhancement coating. The Zr metal coating can be applied by PVD, CVD, ALD and PE-CVD in an inert environment. The coating thickness of Zr metal is 0.5-15 microns. Thereafter, the Zr metal coating is exposed to nitrogen gas and/or a nitrogen containing gas compound and then to carbon and/or a carbon containing gas compound (e.g., methane and/or acetylene gas). The color of the ZrNC will vary depending on the amount of C and N in the coating. In one non-limiting embodiment, the enhancement coating composition generally includes 40-95 wt. % Zr (and all values and ranges therebetween), 5-40 wt. % N (and all values and ranges therebetween), and 5-40 wt. % C (and all values and ranges therebetween), 0-2 wt. % O (and all values and ranges therebetween), 0-10 wt. % Re (and all values and ranges therebetween), and 0-20 wt. % Si (and all values and ranges therebetween). In another non-limiting embodiment, the enhancement coating composition generally includes 40-80 wt. % Zr, 5-25 wt. % N, and 5-25 wt. % C, 0-1 wt. % O, 0-8 wt. % Re, and 0-1 wt. % Si.

In non-limiting configuration, a portion or all of the medical device is formed of a metal alloy that includes a) stainless steel, b) CoCr alloy, c) TiAIV alloy, d) aluminum alloy, e) nickel alloy, f) titanium alloy, g) tungsten alloy, h) molybdenum alloy, i) copper alloy, j) beryllium-copper alloy, k) titanium-nickel alloy, 1) refractory metal alloy, or m) metal alloy (e.g., stainless steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, etc.) that includes at least 5 awt. % rhenium and/or hafnium, and wherein the metal alloy is coated with a metal oxynitride layer (e.g., titanium nitride oxide and/or (TiNOx), zirconium oxynitride (ZrNxOy), etc.), which metal oxynitride layer can optionally be used to promotes and/or facilitates in a) formation or generation of nitric oxide (NO), b) stimulation of endothelial cells, c) a modulation of endothelial cells, d) reduce neointimal hyperplasia, e) reduce tissue proliferation, f) reduce platelet activation, g) reduce thrombosis, h) reduce restenosis, i) promote endothelial cell angiogenesis, and/or j) improved healing on and/or about the medical device, and wherein the outer surface of the metal alloy optionally includes an adhesion layer, which adhesion layer is optionally a metallic layer that includes titanium or zirconium.

In another non-limiting configuration, the medical device is a stent or a prosthetic heart valve, and wherein all or a portion of the stent or frame of the prosthetic heart valve is formed of a titanium-nickel alloy or a titanium-nickel alloy that includes at least 5 awt. % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components other than the frame of the prosthetic heart valve (e.g., leaflet, skirts, etc.) are optionally coated with a metal oxynitride layer, and wherein the outer surface of the metal alloy optionally includes an adhesion layer, which adhesion layer is optionally a metallic layer that includes titanium or zirconium.

In another non-limiting configuration, the medical device is a stent or a prosthetic heart valve, and wherein all or a portion of the stent or frame of the prosthetic heart valve is formed of a stainless-steel alloy or a stainless-steel alloy that includes at least 5 awt % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components other than the frame of the prosthetic heart valve (e.g., leaflet, skirts, etc.) are optionally coated with a metal oxynitride layer, and wherein the outer surface of the metal alloy optionally includes an adhesion layer, which adhesion layer is optionally a metallic layer that includes titanium or zirconium.

In another non-limiting configuration, the medical device is a stent or a prosthetic heart valve, and wherein all or a portion of the stent or frame of the prosthetic heart valve is formed of a cobalt-chromium alloy or a cobalt-chromium alloy that includes at least 5 awt % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components other than the frame of the prosthetic heart valve (e.g., leaflet, skirts, etc.) are optionally coated with a metal oxynitride layer, and wherein the outer surface of the metal alloy optionally includes an adhesion layer, which adhesion layer is optionally a metallic layer that includes titanium or zirconium.

In another non-limiting configuration, the medical device is a stent or a prosthetic heart valve, and wherein all or a portion of the stent or frame of the prosthetic heart valve is formed of a TiAlV alloy or a TiAlV alloy that includes at least 5 awt % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components other than the frame of the prosthetic heart valve (e.g., leaflet, skirts, etc.) are optionally coated with a metal oxynitride layer, and wherein the outer surface of the metal alloy optionally includes an adhesion layer, which adhesion layer is optionally a metallic layer that includes titanium or zirconium.

In another non-limiting configuration, the medical device is a stent or a prosthetic heart valve, and wherein all or a portion of the stent or frame of the prosthetic heart valve is formed of a refractory metal alloy or a refractory metal alloy that includes at least 5 awt % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components other than the frame of the prosthetic heart valve (e.g., leaflet, skirts, etc.) are optionally coated with a metal oxynitride layer, and wherein the outer surface of the metal alloy optionally includes an adhesion layer, which adhesion layer is optionally a metallic layer that includes titanium or zirconium.

In another non-limiting configuration, the medical device is a stent or a prosthetic heart valve, and wherein all or a portion of the stent or frame of the prosthetic heart valve is formed of a metal alloy that includes at least 5 awt % rhenium and/or hafnium, and wherein a portion or all of the outer surface of the metal alloy is coated with a metal oxynitride layer (e.g., titanium nitride oxide and/or (TiNOx), zirconium oxynitride (ZrNxOy), etc.), and wherein all or a portion of components other than the frame of the prosthetic heart valve (e.g., leaflet, skirts, etc.) are optionally coated with a metal oxynitride layer, and wherein the outer surface of the metal alloy optionally includes an adhesion layer, which adhesion layer is optionally a metallic layer that includes titanium or zirconium.

In accordance with another and/or alternative non-limiting aspect of the present disclosure, the metal alloy that includes at least 5 atw. % rhenium and/or hafnium or a refractory metal alloy optionally has reduced ion release of the primary components of the metal alloy as compared to stainless steel, cobalt-chromium alloy, nickel-titanium alloy, or TiAlV alloy. In one non-limiting embodiment, and wherein the metal alloy that includes at least 5 atw. % rhenium and/or hafnium or refractory metal alloy has a maximum ion release of a primary component of the metal alloy when inserted or implanted on or in the body of the patient of no more than 0.5 μg/cm² per day (e.g., 0.001-0.5 μg/cm² per day and all values and ranges therebetween); and wherein the primary component is a component of the metal alloy that constitutes at least 2 wt. % of the metal alloy; and wherein the metal alloy optionally has an absolute increase in ion release per dose of metal alloy in the tissue about the implanted medical device of no more than 50 days (e.g., 5-50 days and all values and ranges therebetween) after inserted or implanted on or in the body of a patient; and wherein the metal alloy optionally has no more than 50% (e.g., 0-50% and all values and ranges therebetween) of the allowed daily exposure of primary metal form the metal alloy during the first 5 days after inserted or implanted on or in the body of a patient, and optionally has no more than 20% (e.g., 0-20% and all values and ranges therebetween) of the allowed daily exposure of primary metal form the metal alloy after the first 5 days after inserted or implanted on or in the body of a patient. As such, the metal alloy that includes at least 5 atw. % rhenium and/or hafnium or a refractory metal alloy optionally results in less potentially irritating metal ions (e.g., nickel ions, chromium ions, etc.) that are released from the metal alloy as compared to ion release from stainless steel, cobalt-chromium alloy, nickel-titanium alloy, or TiAlV alloy.

In accordance with another and/or alternative non-limiting aspect of the present disclosure, the metal alloy that includes at least 5 atw. % rhenium and/or hafnium or a refractory metal alloy optionally has increased hydrophilicity as compared to stainless steel, cobalt-chromium alloy, nickel-titanium alloy, or TiAlV alloy. Hydrophilicity of a material implanted in a patient is an important property of the material with regard to the cell adhesion, cell migration, and cell multiplication of tissue on the material. In one non-limiting embodiment, the metal alloy that includes at least 5 atw. % rhenium and/or hafnium or a refractory metal alloy optionally has a hydrophilicity wherein a contact angle of a water droplet on a surface of said metal alloy of 25-45° (e.g., 0.1-4.99 and all values and ranges therebetween). As a comparison, CoCr alloys are hydrophobic materials resulting in a large contact angle (93°±1°) of a water droplet (e.g., distilled water) positioned on the surface of the CoCr alloy. TiAlV alloys are a little more hydrophilic than CoCr alloys and exhibit a contact angle of 58°±8° when a water droplet is positioned on the surface of the Ti alloy. Metal alloys such as a MoRe alloy have a much greater hydrophilicity and have a contact angle of 37°±3° when a water droplet is positioned on the surface of the MoRe alloy.

In accordance with another and/or alternative non-limiting aspect of the present disclosure, there is provided a non-limiting method for forming a portion or all of an expandable stent or an expandable frame for a medical device that includes the steps of a) providing metal alloy powder; and wherein the metal alloy power optionally has an average particle size of less than 200 mesh (e.g., 200-635 mesh and all values and ranges therebetween; 20-74 microns and all values and ranges therebetween); and wherein the purity of the metal alloy power is optionally at least 90% (e.g., 90-100% purity and all values and ranges therebetween); b) consolidating the metal alloy powder into a general shape of a rod; and wherein the step of consolidating optionally includes subjecting the metal alloy powder to an isostatic process that optionally applies a uniform pressure of 50-700 MPa (and all values and ranges therebetween) from all sides on the metal powder; and wherein the step of consolidating optionally occurs in an inert atmosphere, an oxygen reducing atmosphere of hydrogen or an argon and hydrogen mixture, and/or under a vacuum; c) sintering the rod shaped consolidated metal alloy powder to form a rod green part; and wherein the rod green part optionally has an average density of 0.7-0.95 (and all values and ranges therebetween) of a minimum theoretical density; and wherein the rod green part optionally has an average density of 10-20 gm/cc (and all values and ranges therebetween); and wherein the consolidated rod-shaped metal alloy powder is optionally sintered at a temperature of 1000-3500° C. (and all values and ranges therebetween) to partially or fully fuse the metal alloy powder together to form the rod green part; and wherein the step of sintering optionally occurs in an inert atmosphere, an oxygen reducing atmosphere of hydrogen or an argon and hydrogen mixture, and/or under a vacuum; d) optionally subjecting the rod green part to a primary reduction process (e.g., swaging process, etc.) to reduce the original outer cross-sectional area of the rod green part to a first drawn down cross-sectional area by use of a reducing mechanism; the rod green part is drawn down one or more times in the reducing mechanism to a first drawn down cross-sectional area of the rod green part; and wherein the first drawn down cross-sectional area of the rod green part is optionally no more than 50% (e.g., 1-50% and all values and ranges therebetween) of the original outer cross-sectional area of the rod green part; and wherein the metal alloy rod after the primary secondary reduction process optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the step of optionally subjecting the rod green part to a primary reduction process optionally occurs in an inert atmosphere, an oxygen reducing atmosphere of hydrogen or an argon and hydrogen mixture, and/or under a vacuum; e) optionally subjecting the rod green part to one or more secondary reduction process to further reduce the outer cross-sectional area of the rod green part to a drawn down cross-sectional area that is less than the cross-sectional area obtained by the primary drawn down process by use of a reducing mechanism; the rod green part is drawn down one or more times in the reducing mechanism to a second, third fourth, etc. drawn down cross-sectional area of the rod green part; and wherein the second, third fourth, etc. drawn down cross-sectional area of the rod green part is optionally no more than 40% (e.g., 1-40% and all values and ranges therebetween) of the primary outer cross-sectional area of the rod green part or some subsequent drawn down outer cross-sectional area of the rod green part; and wherein the metal alloy rod after the final secondary reduction process optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the step of optionally subjecting the rod green part to one or more secondary reduction process optionally occurs in an inert atmosphere, an oxygen reducing atmosphere of hydrogen or an argon and hydrogen mixture, and/or under a vacuum; wherein the one or more secondary reduction processes optionally includes a swaging process; and wherein the swaging process optionally occurs at a temperature of 400° C.-1500° C. (and all values and ranges therebetween) in a controlled neutral or non-reducing environment (e.g., an inert atmosphere, an oxygen reducing atmosphere of hydrogen or an argon and hydrogen mixture, under a vacuum, etc.); f) optionally annealing the rod green part after the rod green part has obtained primary drawn down process and/or after the rod green part has obtained one or more secondary reduction process; and wherein the optional annealing step can optionally occur after certain drawing downs of the outer cross-sectional area of the rod green part, or after all but the last drawing down of the outer cross-sectional area of the rod green part, or after all of the drawing downs of the outer cross-sectional area of the rod green part; and wherein the step of annealing optionally includes controlling an atmosphere (e.g., an inert atmosphere, an oxygen reducing atmosphere of hydrogen or an argon and hydrogen mixture, under a vacuum, etc.) about the rod green part during the step of annealing so that the rod green part after the step of annealing optionally includes no more than 30 ppm nitrogen (0-30 ppm and all values and ranges therebetween), optionally includes no more than 150 ppm carbon (0-150 ppm and all values and ranges therebetween), optionally includes no more than 100 ppm oxygen (0-100 ppm and all values and ranges therebetween), and optionally has a carbon to oxygen atomic ratio of at least about 0.2:1 (e.g. 0.2:1 to 50:1 and all values and ranges therebetween); and wherein the annealing temperature during the step of annealing is optionally greater than 500° C. (e.g. 500-1600° C. and all values and ranges therebetween); and wherein the optional step of annealing or the optional step of reduction of cross-sectional area forms a metal alloy rod form the rod green part; g) subjecting the metal alloy rod to a gun-drilling process and/or an EDM cutting process to form a metal alloy tube from the metal alloy rod; and wherein the step of gun-drilling and/or EDM cutting optionally includes controlling an atmosphere (e.g., an inert atmosphere, an oxygen reducing atmosphere of hydrogen or an argon and hydrogen mixture, under a vacuum, etc.) about the metal alloy tube from the metal alloy rod during the step of gun-drilling and/or EDM cutting; and h) cutting, etching, grinding, laser cutting, and/or shaving the metal alloy tube to partially or fully form the expandable stent or the expandable frame for a medical device; and wherein the step of cutting, etching, grinding, laser cutting, and/or shaving the metal alloy tube optionally includes controlling an atmosphere (e.g., an inert atmosphere, an oxygen reducing atmosphere of hydrogen or an argon and hydrogen mixture, under a vacuum, etc.) about the metal rod during the step of cutting, etching, grinding, laser cutting, and/or shaving the metal alloy tube.

In accordance with another and/or alternative non-limiting aspect of the present disclosure, there is provided a non-limiting method step for forming the expandable stent or the expandable frame for a medical device wherein after the final optional secondary reduction process the metal alloy rod is not exposed to a heat treatment process that includes exposing the metal alloy rod a temperature of 500° C. or more.

In accordance with another and/or alternative non-limiting aspect of the present disclosure, there is provided a non-limiting method step for forming the expandable stent or the expandable frame for a medical device wherein one or more outer surfaces of the metal alloy are optionally subjected to a nitriding process a) prior to and/or after the primary reduction process, b) prior to and/or after the one or more optional secondary reduction processes, c) prior to and/or after the optional annealing process, d) prior to and/or after to the optionally subjecting the metal alloy rod to a gun-drilling process and/or an EDM cutting process to form a metal alloy tube from the metal alloy rod, and/or e) prior to and/or after to the optionally cutting, etching, grinding, laser cutting, and/or shaving the metal alloy rod or metal alloy tube to partially or fully form the medical device.

In accordance with another and/or alternative non-limiting aspect of the present disclosure, there is provided a non-limiting method step for forming the expandable stent or the expandable frame for a medical device wherein one or more outer surfaces of the metal alloy are optionally coated with an agent, and optionally a polymer to optionally control the release rate of the agent.

In accordance with another and/or alternative non-limiting aspect of the present disclosure, there is provided a non-limiting method step for forming the expandable stent or the expandable frame for a medical device wherein one or more outer surfaces of the metal alloy are optionally coated with an enhancement coating.

In accordance with another and/or alternative non-limiting aspect of the present disclosure, there is provided a non-limiting method step for forming the expandable stent or the expandable frame for a medical device wherein the metal alloy optionally has reduced ion release of the primary components of the metal alloy as compared to stainless steel, cobalt-chromium alloy, nickel-titanium alloy, or TiAlV alloy; and wherein the metal alloy optionally has a maximum ion release of a primary component of the metal alloy when inserted or implanted on or in the body of the patient of no more than 0.5 μg/cm² per day (e.g., 0.001-0.5 μg/cm² per day and all values and ranges therebetween); and wherein the primary component is a component of the metal alloy that constitutes at least 2 wt. % of the metal alloy; and wherein the metal alloy optionally has an absolute increase in ion release per dose of metal alloy in the tissue about the implanted medical device of no more than 50 days (e.g., 5-50 days and all values and ranges therebetween) after inserted or implanted on or in the body of a patient; and wherein the metal alloy optionally has no more than 50% (e.g., 0-50% and all values and ranges therebetween) of the allowed daily exposure of primary metal form the metal alloy during the first 5 days after inserted or implanted on or in the body of a patient, and optionally has no more than 20% (e.g., 0-20% and all values and ranges therebetween) of the allowed daily exposure of primary metal form the metal alloy after the first 5 days after inserted or implanted on or in the body of a patient.

In accordance with another and/or alternative non-limiting aspect of the present disclosure, there is provided a non-limiting method step for forming the expandable stent or the expandable frame for a medical device wherein after the metal alloy tube is cut (e.g., laser cut, etc.) to partially or fully form the configuration of the final shape of the medical device, the cut metal alloy tube is optionally submitted to one or more of the following steps: a) heat treating the cut metal tube at a temperature of at least 500° C. (e.g., 500° C.-1500° C. and all values and ranges therebetween), and wherein the heat treatment optionally occurs in a non-oxidizing environment (e.g., hydrogen environment, argon environment, under a vacuum, an inert atmosphere, an argon and hydrogen mixture, etc.), b) deburr dross and other undesired material from the interior of the cavity in the cut metal alloy tube, c) electropolish an outer surface and/or inner cavity surface of the cut metal alloy tube, and/or d) expand the cut metal alloy tube to an expanded configuration or orientation and thereafter subject the expanded cut metal alloy tube to one or more of the following processing steps: I) heat treating the expanded cut metal tube at a temperature of at least 500° C. (e.g., 500° C.-1500° C. and all values and ranges therebetween), and wherein the heat treatment optionally occurs in a non-oxidizing environment (e.g., hydrogen environment, argon environment, under a vacuum, an inert atmosphere, an argon and hydrogen mixture, etc.), II) electropolish an outer surface and/or inner cavity surface of the expanded cut metal alloy tube, and/or III) coat the outer surface of the expanded cut metal alloy tube with an enhancement coating.

In accordance with another and/or alternative non-limiting aspect of the present disclosure, there is provided a non-limiting method step for forming the expandable stent or the expandable frame for a medical device wherein metal alloy optionally has increased hydrophilicity as compared to stainless steel, cobalt-chromium alloy, nickel-titanium alloy, or TiAlV alloy, and wherein the metal alloy optionally has a hydrophilicity wherein a contact angle of a water droplet on a surface of the metal alloy is 25-45° (e.g., 0.1-4.99 and all values and ranges therebetween).

In accordance with another and/or alternative non-limiting aspect of the present disclosure, there is provided a non-limiting method step for forming the expandable stent or the expandable frame for a medical device includes:
a) providing metal powder that includes one or more metals selected from the group consisting of calcium, chromium, cobalt, copper, gold, hafnium, iron, lead, magnesium, molybdenum, nickel, niobium, osmium, platinum, rare earth metals, rhenium, silver, tantalum, technetium, titanium, tungsten, vanadium, yttrium, zinc, and zirconium. The average particle size of the metal powder optionally has a size than is less than 200 mesh (e.g., 200-635 mesh and all values and ranges therebetween and all values and ranges therebetween). The metal powder optionally has a purity of at least 90% (e.g., 90-100% purity and all values and ranges therebetween). The metal powder can be a pure metal powder and/or a metal alloy powder.
b) mixing the metal powder to provide a homogeneous and uniform mixture of the metal powder. The metal powder is sufficiently mixed so as to obtain a specific ratio of metals of the metal powder to obtain the intended metal alloy that is to be used to partially or fully form the medical device. The process for mixing the metal powder is non-limiting. The metal powder can optionally be mixed in an inert atmosphere, an oxygen reducing atmosphere of hydrogen or an argon and hydrogen mixture, and/or under a vacuum.
c) consolidating the homogeneous and uniform mixture of metal powders into a rod-shaped green part. The outer diameter and length of the rod-shaped green part is non-limiting. In one non-limiting configuration, the rod-shaped green part has a constant cross-sectional shape and constant cross-sectional area along 10-100% (and values and ranges therebetween) the longitudinal length of the rod-shaped green part. The step of consolidating optionally includes subjecting the homogeneous and uniform mixture of metal powder to an isostatic process. In one non-limiting configuration, the isostatic process optionally applies a uniform pressure of at least 70 MPa (e.g., 70-1000 MPa and all values and ranges therebetween) from one or more or all sides on the homogeneous and uniform mixture of metal powder to form the rod-shaped green part. The rod-shaped green part can have a variety of cross-sectional shapes (e.g., circular shaped, oval shaped, square shaped, rectangular shaped, triangular shaped, polygonal shapes, etc.). During the step of consolidation, the metal powder in the rod-shaped green part is typically not alloyed and/or sintered together. As such, the rod-shaped green part generally has a low structure integrity. The rod-shaped green part generally has a density of less than 80% of a density of a same metal alloy rod that is formed by casting. The consolidating of the metal powder to form the rod-shaped green part can optionally occur in an inert atmosphere, an oxygen reducing atmosphere of hydrogen or an argon and hydrogen mixture, and/or under a vacuum.
d) sintering the rod-shaped green part to fuse together a portion of all of the metal powder in said rod-shaped green part to form a semi-dense metal alloy rod having an original cross-sectional area. The density of the metal alloy rod is generally more dense than the rod-shaped green part (e.g., 5-200% more dense and all values and ranges therebetween). The metal alloy rod can optionally has a density of 40-98% of a density of a same metal alloy rod that is formed by casting after the step of sintering. The step of sintering generally occurs at a temperature of at least 800° C. (e.g., 800-3500° C. and all values and ranges therebetween) to partially or fully fuse together the metal powder in the rod-shaped green part to form the metal alloy rod. The metal alloy rod optionally has an average density of 5-20 gm/cc (e.g., and all values and ranges therebetween) after the step of sintering. The step of sintering can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the step of sintering.
e) subjecting the metal alloy rod to a primary reduction process to form a metal alloy rod that has a first drawn-down cross-sectional area that is less than the original outer cross-sectional area of the metal alloy rod after the sintering step. The primary reduction process optionally occurs at a temperature of 400-1900° C. (and all values and ranges therebetween). The metal alloy rod can be drawn down in cross-sectional area one or more times during the primary reduction process. In one non-limiting configuration, the metal alloy rod is continuously heated during the one or more times of drawing down during the primary reduction process. The first drawn down cross-sectional area optionally reduces the original outer cross-sectional area of the rod-shaped green part by no more than 50% (e.g., 5-50% and all values and ranges therebetween). The step of primary reduction process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the primary reduction process. The metal alloy rod after the primary reduction process has a density of greater than 80% (e.g., 80-95% and all values and ranges therebetween) of a density of a same metal alloy rod that is formed by casting. The primary reduction process optionally includes the use of a swagging process.
f) subjecting the metal alloy rod to one or more secondary process steps after the primary reduction process, wherein the one or more secondary process steps includes one or more steps selected from the group consisting of I) subjecting the metal alloy rod to a gun drilling process and/or an EDM cutting process to form a metal alloy tube from the metal alloy rod; II) cutting, etching, grinding, laser cutting, and/or shaving the metal alloy rod or the metal alloy tube to partially or fully form the medical device; III) subjecting the metal rod to a secondary reduction process and/or a final reduction process to reduce the cross-sectional area of the metal alloy rod to less than the first drawn down cross-sectional area; IV) subjecting one or more portions of an outer surface of the metal alloy rod or metal alloy tube to a nitriding process; V) coating one or more portions of an outer surface of the metal alloy rod or metal alloy tube with an agent and/or polymer; VI) coating one or more portions of an outer surface of the metal alloy rod or metal alloy tube with an enhancement coating, and/or VII) annealing the metal alloy rod after the primary reduction process and/or after the secondary reduction process.

In accordance with another and/or alternative non-limiting aspect of the present disclosure, the metal rod is optionally subjected to one or more annealing processes. The one or more annealing processes can occur after the primary reduction process and/or after the secondary reduction process. The step of annealing can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the step of annealing. The temperature during the step of annealing is optionally at least 500° C.

In accordance with another and/or alternative non-limiting aspect of the present disclosure, the metal rod is optionally subjected to a secondary reduction process to reduce the first drawn-down cross-sectional area to a second drawn-down cross-sectional area, and wherein the second drawn-down cross-sectional area is less than the first drawn-down cross-sectional area. The secondary reduction process generally occurs either a) after the metal alloy rod has been drawn down by 50% of the original outer cross-sectional area of the rod-shaped green part or b) after the first annealing of the metal alloy rod. The secondary reduction process optionally occurs at a temperature of 400-1900° C. (and all values and ranges therebetween). The metal alloy rod can be drawn down in cross-sectional area one or more times during the secondary primary reduction process. In one non-limiting configuration, the metal alloy rod is continuously heated during the one or more times of drawing down during the secondary reduction process. During each draw down during the secondary reduction process, the metal alloy rod is not reduced in cross-section by more than 40% (e.g., 2-40% and all values and ranges therebetween). During the secondary reduction process, the metal alloy rod can optionally be annealed one or more times. The secondary reduction process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the secondary reduction process. The metal alloy rod after the secondary reduction process has a density of greater than 90% (e.g., 90-99.99% and all values and ranges therebetween) of a density of a same metal alloy rod that is formed by casting. The metal alloy rod optionally has a higher density after the secondary reduction process than the density of the metal alloy rod after the primary reduction process. The secondary reduction process optionally includes the use of a swagging process.

In accordance with another and/or alternative non-limiting aspect of the present disclosure, the metal rod is optionally subjected to final reduction process to reduce cross-sectional area of the metal rod. The metal alloy rod after the final reduction process has a density of greater than 90% (e.g., 90-99.99% and all values and ranges therebetween) of a density of a same metal alloy rod that is formed by casting. The metal alloy rod optionally has a higher density after the final reduction process than the density of the metal alloy rod after the primary reduction process. The final reduction process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the final reduction process. The metal alloy rod after said final reduction process optionally has one or more of i) at least 125 Ksi yield strength, ii) at least 130 Ksi ultimate strength, iii) an elongation of at least 9%, and/or iv) a reduction in cross-sectional area from said original cross-sectional area of at least 40%. The final reduction process optionally includes the use of a swagging process.

In accordance with another and/or alternative non-limiting aspect of the present disclosure, the metal rod is optionally subjected to a gun drilling process and/or an EDM cutting process to form a metal alloy tube from the metal alloy rod. The gun drilling process and/or an EDM cutting process can optionally occur in an inert atmosphere, an oxygen reducing atmosphere of hydrogen or an argon and hydrogen mixture, and/or under a vacuum. The metal alloy rod optionally includes no more than 30 ppm nitrogen, no more than 150 ppm carbon (e.g., 0-150 ppm carbon and all values and ranges therebetween), no more than 30 ppm nitrogen (e.g., 0-30 ppm nitrogen and all values and ranges therebetween), and no more than 100 ppm oxygen (e.g., 0-150 ppm oxygen and all values and ranges therebetween) after the gun drilling process and/or an EDM cutting process.

In accordance with another and/or alternative non-limiting aspect of the present disclosure, the metal rod is optionally subjected to cutting, etching, grinding, laser cutting, and/or shaving the metal alloy rod or the metal alloy tube to partially or fully form the medical device. In one non-limiting embodiment, the medical device is selected from a stent, medical device frame, or hypotube.

In accordance with another and/or alternative non-limiting aspect of the present disclosure, the metal rod is optionally subjected to a nitriding process a) prior to and/or after the primary reduction process, b) prior to and/or after the secondary reduction process, c) prior to and/or after the final reduction process, d) prior to and/or after the annealing process, e) prior to and/or after the gun drilling process and/or said EDM cutting process, and/or f) prior to and/or after the cutting, etching, grinding, laser cutting, and/or shaving of the metal alloy rod or metal alloy tube.

In accordance with another and/or alternative non-limiting aspect of the present disclosure, one or more portions of an outer surface of the metal alloy rod or metal alloy tube is coated with an agent and/or polymer. In one non-limiting embodiment, there is provided a metal alloy tube that is configured to be expandable, and wherein the expandable metal alloy tube is expanded prior to coating the expandable metal alloy tube with the agent and/or polymer.

In accordance with another and/or alternative non-limiting aspect of the present disclosure, one or more portions of an outer surface of the metal alloy rod or metal alloy tube is coated with an enhancement coating. The enhancement coating optionally includes one or more coatings selected from the group consisting of chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZrNxOy), oxyzirconium-nitrogen-carbon (ZrNC), and/or zirconium OxyCarbide (ZrOC); said enhancement coating coated on said metal alloy rod or said metal alloy tube by one or more processes selected from the group consisting of physical vapor deposition (PVD) process, chemical vapor deposition (CVD) process, atomic layer deposition (ALD) process, or a plasma-enhanced chemical vapor deposition (PE-CVD) process. In one non-limiting embodiment, there is provided a metal alloy tube that is configured to be expandable, and wherein the expandable metal alloy tube is expanded prior to coating the expandable metal alloy tube with the enhancement coating.

In accordance with another and/or alternative non-limiting aspect of the present disclosure, the metal alloy that is used to partially or fully form the metal alloy rod or metal alloy tube has a) a maximum ion release of a primary component of the metal alloy when inserted or implanted on or in a body of a patient of no more than 0.5 μg/cm² per day (e.g., 0.0001-0.5 g/cm² per day and all values and ranges therebetween); and wherein the primary component is a component of the metal alloy that constitutes at least 2 wt. % of the metal alloy; b) the metal alloy has an absolute increase in ion release per dose of the metal alloy in the tissue about the implanted medical device of no more than 50 days (e.g. 0.5-50 days and all values and ranges therebetween) after inserted or implanted on or in the body of the patient; c) the metal alloy has no more than 50% (e.g., 0-50% and all values and ranges therebetween) of the allowed daily exposure of primary metal from the metal alloy during the first 5 days after inserted or implanted on or in the body of the patient; and/or d) a hydrophilicity wherein a contact angle of a water droplet on a surface of the metal alloy is 25-45° (and all values and ranges therebetween).

In accordance with another and/or alternative non-limiting aspect of the present disclosure, the metal alloy is formed of I) at least 10-15 awt. % hafnium and/or rhenium and 50-78 wt. % iron, and one or more of a) 9-27 wt. % chromium, b) 0.1-26 wt. % nickel, c) 0.01-7 wt. % molybdenum, d) 0.01-16 wt. % manganese, e) 0.01-4 wt. % silicon, f) 0.01-2 wt. % titanium, g) 0.01-1 wt. % selenium, h) 0.01-1 wt. % niobium, i) 0.01-2 wt. % aluminum, j) 0.01-1 wt. % tantalum, k) 0.01-1 wt. % cobalt, 1) 0.01-5 wt. % copper, m) 0.01-1 wt. % vanadium, and n) 0.01-2 wt. % tungsten; or II) at least 10-15 awt. % hafnium and/or rhenium and 35-68 wt. % cobalt, and one or more of a) 12-28 wt. % chromium, b) 0.01-38 wt. % nickel, c) 0.1-30 wt. % molybdenum, d) 0.01-2 wt. % manganese, e) 0.01-1 wt. % silicon, f) 0.01-18 wt. % tungsten, g) 0.01-0.5 wt. % lanthanum, h) 0.01-20 wt. % iron, i) 0.01-5 wt. % titanium, j) 0.01-2 wt. % niobium, k) 0.01-2 wt. % aluminum, 1) 0.01-1 wt. % silicon, m) 0.01-0.5 wt. % boron, and n) 0.01-0.5 wt. % silver; or III) at least 10-15 awt. % hafnium and/or rhenium and 70-91.5 wt. % titanium, and one or more of a) 2-8 wt. % aluminum, b) 0.01-16 wt. % vanadium, c) 0.01-1 wt. % iron, d) 0.01-0.5 wt. % yttrium, e) 0.01-20 wt. % chromium, f) 0-16 wt. % molybdenum, g) 0.01-2 wt. % nickel, h) 0.01-12 wt. % tin, i) 0.01-6 wt. % zirconium, j) 0.01-2 wt. % tantalum, k) 0.01-4 wt. % niobium, 1) 0.01-1 wt. % silicon, and m) 0.01-3 wt. % iron; or IV) at least 10-15 awt. % hafnium and/or rhenium, 35-84 wt. % tantalum, and one or more of a) 0.1-25 wt. % tungsten, b) 0.1-30 wt. % molybdenum, c) 0.01-45 wt. % niobium, d) 0.01-5 wt. % chromium, f) 0.01-5 wt. % titanium, g) 0.01-5 wt. % zirconium, and h) 0.01-4 wt. % hafnium; or V) at least 10-15 awt. % hafnium and/or rhenium, 40-85 wt. % niobium, and one or more of a) 0.01-20 wt. % molybdenum, b) 0.01-35 wt. % tantalum, c) 0.01-12 wt. % hafnium, d) 0.01-5 wt. % zirconium, e) 0.01-3 wt. % titanium, f) 0.01-15 wt. % tungsten, and g) 0.01-1 wt. % yttrium; or VI) at least 10-15 awt. % rhenium, 30-58 wt. % titanium, and 30-58 wt. % nickel; or VII) at least 10-15 awt. % hafnium and/or rhenium, and one or more of a) 1-85 awt. % chromium, b) 0.1-10 awt. % titanium, c) 0.1-10 awt. % molybdenum, and d) 0.1-10 awt. % zirconium; or VIII) at least 10-15 awt. % hafnium and/or rhenium, 15-32 wt. % chromium, 1-36% wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % aluminum, 0-1 wt. % iron, 30-68 wt. % cobalt; or IX) at least 10-15 awt. % hafnium and/or rhenium, 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt; or X) at least 10-15 awt. % hafnium and/or rhenium, 38-42 wt. % cobalt, 18-22 wt. % chromium, 14-18 wt. % iron, 13-17 wt. % nickel, 6-8 wt. % molybdenum; or XI) at least 10-15 awt. % hafnium and/or rhenium, 18-22 wt. % chromium, 14-16 wt. % tungsten, 9-11 wt. % nickel, balance cobalt; XII) at least 10-15 awt. % hafnium and/or rhenium, 5.5-6.75 wt. % aluminum, 3.5-4.5 wt. % vanadium, 85-93 wt. % titanium, 0-0.4 wt. % iron, 0-0.2 wt. % carbon; XIII) at least 10-15 awt. % hafnium and/or rhenium, 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium; XIV) at least 10-15 awt. % hafnium and/or rhenium, 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper; XV) at least 10-15 awt. % hafnium and/or rhenium, 30-98 wt. % nickel, 5-25 wt. % chromium, 0-65 wt. % iron, 0-30 wt. % molybdenum, 0-32% wt. % copper, 0-32% wt. % cobalt, 2-2 wt. % aluminum, 0-6 wt. % tantalum, 0-15% wt. % tungsten, 0-5 wt. % titanium, 0-6 wt. % niobium, 0-3 wt. % silicon; XVI) at least 10-15 awt. % hafnium and/or rhenium, 80-99 wt. % titanium, 0-6 wt. % aluminum, 0-3 wt. % tin, 0-1 wt. % palladium, 0-8 wt. % vanadium, 0-15% wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron; XVII) at least 10-15 awt. % hafnium and/or rhenium, 85-98 wt. % tungsten, 0-8 wt. % nickel, 0-5 wt. % copper, 0-5 wt. % molybdenum, 0-4 wt. % iron; XVIII) at least 10-15 awt. % hafnium and/or rhenium, 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum; XIX) at least 10-15 awt. % hafnium and/or rhenium, 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 wt. % cobalt, 0-20% wt. % nickel; XX) at least 10-15 awt. % hafnium and/or rhenium, 32-38 wt. % nickel, 18-22 wt. % chromium, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. % phosphorous, 0-0.1 wt. % boron, 0-0.1 wt. % sulfur, and cobalt; XXI) at least 10-15 awt. % hafnium and/or rhenium, 95-98.5 wt. % copper, 1-4 wt. % beryllium, 0-1 wt. % cobalt, and 0-0.5 wt. % silicon; XXII) at least 10-15 awt. % hafnium and/or rhenium, 30-98 wt. % rhenium, and optionally one or more 5-25 wt. % chromium, 0-65 wt. % iron, 0-30 wt. % molybdenum, 0-32 wt. % copper, 0-32 wt. % cobalt, 2-2 wt. % aluminum, 0-6 wt. % tantalum, 0-15 wt. % tungsten, 0-5 wt. % titanium, 0-6 wt. % niobium, 0-3 wt. % silicon; XXIII) at least 10-15 awt. % hafnium and/or rhenium, 42-58 wt. % nickel and 42-58 wt. % titanium; XXIV) at least 1 wt. % rhenium and/or hafnium and one or more metal alloying additives selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than rhenium, b) metals other than the one or more the metal alloying additives, c) carbon, d) oxygen, and e) nitrogen; XXV) at least 20 wt. % of primary metal and one or more metal alloying additives, and wherein the primary metal includes one or more metals selected from the group consisting of molybdenum, niobium, hafnium, rhenium, tantalum, and tungsten, and wherein the one or more metal alloying additives includes one or more metals selected from the group consisting of calcium, carbon, chromium, cobalt, copper, gold, iron, magnesium, nickel, niobium, osmium, platinum, rare earth metals, rhenium, silver, tantalum, technetium, titanium, tungsten, vanadium, yttrium, zinc, and zirconium; and wherein the metal alloy includes 0-2 wt. % of a combination of a) metals other than the primary metal, b) metals other than the one or more metal alloying additives, c) carbon, d) oxygen, and e) nitrogen; XXVI) stainless-steel; XXVII) CoCr alloy; XXVIII) TiAIV alloy; XXIX) aluminum alloy; XXX) nickel alloy; XXXI) titanium alloy; XXXII) tungsten alloy; XXXIII) molybdenum alloy; XXXIV) copper alloy; XXXV) beryllium-copper alloy; XXXVI) titanium-nickel alloy; XXXVII) refractory metal alloy; XXXVIII) metal alloy that is formed of stainless-steel, CoCr alloy, TiAIV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) rhenium (e.g., 5-99 awt. % rhenium and all values and ranges therebetween); XXXIX) metal alloy that is formed of stainless-steel, CoCr alloy, TiAlV alloy, aluminum alloy, nickel alloy, titanium alloy, tungsten alloy, molybdenum alloy, copper alloy, beryllium-copper alloy, titanium-nickel alloy, refractory metal alloy, and wherein the metal alloy has been modified to further include at least 5 atomic weight percent (awt. %) or atomic percent (awt. %) hafnium (e.g., 5-99 awt. % hafnium and all values and ranges therebetween).

In accordance with another and/or alternative non-limiting aspect of the present disclosure, there is provided a prosthetic heart valve that includes an expandable frame, a leaflet structure supported by the frame, and an optional outer skirt secured to the outer surface of the frame, optional inner skirt, and/or leaflet structure. The prosthetic heart valve can be implanted in the annulus of the native aortic valve; however, the prosthetic heart valve also can be configured to be implanted in other valves of the heart (e.g., tricuspid valve, pulmonary valve, mitral valve). The prosthetic heart valve has a “lower” or “proximal” end and an “upper” or “distal” end, wherein the lower or proximal end of the prosthetic heart valve is the inflow end and the upper or distal end of the prosthetic heart valve is the outflow end.

In accordance with another and/or alternative non-limiting aspect of the present disclosure, the expandable frame of the prosthetic heart valve is configured to be radially collapsible to a collapsed or crimped state for introduction into the body (e.g., on a delivery catheter, etc.) and radially expandable to an expanded state for implanting the prosthetic heart valve at a desired location in the heart (e.g., the aortic valve, tricuspid valve, pulmonary valve, mitral valve, etc.). The expandable frame of the prosthetic heart valve is formed of a plastically-expandable material that permits crimping of the frame to a smaller profile for delivery and expansion of the expandable frame at the treatment site. The expansion of the crimped frame of the prosthetic heart can be by an expansion device such as, but not limited to, a balloon on a catheter. The expandable frame and/or prosthetic heart valve can optionally be configured to be crimped to a diameter of less than 24 FR (e.g., less than 8 mm, 5-7.9 mm, etc.) and the expandable frame and/or prosthetic heart valve can optionally be configured to be expanded to a diameter of at least 14 mm (e.g., 14-35 mm and all values and ranges therebetween); however, it can be appreciated that the expandable frame and/or prosthetic heart valve can be designed to be crimped to larger diameters, and/or be expanded to larger diameters.

In accordance with another and/or alternative non-limiting aspect of the present disclosure, the expandable frame of the prosthetic heart valve is formed of a plurality of angular articulating members and vertically extending vertically extending axial longitudinal members. The angular articulating members and vertically extending vertically extending axial longitudinal members are interconnected to form a variety of patterns (e.g., zig-zag pattern, saw-tooth pattern, triangular pattern, polygonal pattern, oval pattern, etc.). One or more of the angular articulating members and/or vertically extending vertically extending axial longitudinal members can have the same or different thicknesses and/or cross-sectional shape and/or cross-sectional area.

In accordance with another and/or alternative non-limiting aspect of the present disclosure, the expandable frame has one or more open cells. As defined herein, an open cell is a cell wherein the walls of the cell are formed of one or more angular articulating members and one or more vertically extending vertically extending axial longitudinal members. In one non-limiting configuration, all of the cells in a row of cells of at least one of the rows of cells in the expandable frame have an open cell configuration. In another non-limiting configuration, all of the cells in the expandable frame have an open cell configuration. In another non-limiting the configuration of each of the cells in a row of cells is the same and the configuration of the cells in two of more of the rows of cells is different. In another non-limiting the configuration of each of the cells in a row of cells is the same and the configuration of the cells in adjacent positioned rows of cells is different. In another non-limiting the configuration of the angular articulating members of each of the cells in the row of cells at the proximal end of the expandable frame to not extend below the bottom end of the vertically extending vertically extending axial longitudinal members that terminate at the proximal end of the expandable frame. In another non-limiting the configuration of the cells in the expandable frame, the joint for a first set of angular articulating members of each cell in a row of cells extends toward the joint of a second set of angular articulating members of the same cell in the same row of cells. In another non-limiting the configuration of the cells in the expandable frame, the joint for a first set of angular articulating members of each cell in first row of cells extends toward the joint of a second set of angular articulating members of the same cell in the first row of cells, and in a second row of cell that positioned adjacent to the first row of cells, the joint for a first set of angular articulating members of each cell in the second row of cells extends away from the joint of a second set of angular articulating members of the same cell in the second row of cells. In another non-limiting the configuration of the cells in the expandable frame, the joint for a first set of angular articulating members that form the distal portion of each cell in the distal row of cells extends toward the joint of a second set of angular articulating members that form the proximal portion of each cell in the proximal row of cells. In another non-limiting the configuration of the cells in the expandable frame, cells in adjacently positioned cell rows are aligned with one another along the longitudinal axis of the expandable frame. In another non-limiting configuration, all of the vertically extending vertically extending axial longitudinal members in adjacently positioned cell rows are aligned along the central longitudinal axis of the vertically extending vertically extending axial longitudinal members. In another non-limiting configuration, each set of angular articulating members in each of the cells are connected at each end to a vertically extending vertically extending axial longitudinal member.

In accordance with another and/or alternative non-limiting aspect of the present disclosure, one or more of the angular articulating members are configured to have a variable cross-sectional area along a longitudinal length of the angular articulating member. In one non-limiting embodiment, each of the angular articulating members have first and second articulating segments that are connected together by an articulating joint, and wherein one or both articular segments of one or more of the angular articulating members has a first cross-sectional area at a proximal region, a second cross-sectional area at a central region, and a third cross-sectional area at a distal region, and wherein a) the minimum second cross-sectional area at the central region is less than a maximum first cross-sectional area at the proximal region, b) the minimum second cross-sectional area at the central region is less than a maximum third cross-sectional area at the distal region, and/or c) the maximum first cross-sectional area at the proximal region is less than a maximum third cross-sectional area at the distal region; and wherein the proximal end of the angular articulating member connects to a vertically extending vertically extending axial longitudinal member and the distal end of the angular articulating member connects to a distal end of another angular articulating member. In another non-limiting embodiment, a plurality of cells include one or both of the first and second articulating segments of the one or more of the angular articulating members that have a taper T that is the same or is the same within 3% (e.g., 0-3% and all values and ranges therebetween) of one another based the taper formula of T=(V−C)/(L/2), wherein T is taper, V is the valley strut width (i.e., articulating joint between two articulating segments), C is the center strut width (i.e., width of the center of an articulating segment), and L is the length of an articulating segment. In one non-limiting configuration, the taper T is 0.01-0.06 (and all values and ranges therebetween).

In accordance with another and/or alternative non-limiting aspect of the present disclosure, one or more of the angular articulating members of one or more cells of the expandable frame have a non-linear shaped central region (e.g., wavy region, etc.). In one non-limiting configuration, all of the angular articulating members in all of the cells of the expandable frame have a non-linear shaped central region (e.g., wavy region, etc.).

In accordance with another and/or alternative non-limiting aspect of the present disclosure, the articulating joint that forms the connection between two angular articulating members of one or more sets angular articulating members have a C-shape or U-shape or curvilinear shape when the expandable frame is in both an expanded and unexpanded configuration. In another non-limiting embodiment, all of the articulating joints that form the connection between two angular articulating members of the sets angular articulating members have a C-shape or U-shape or curvilinear shape when the expandable frame is in both an expanded and unexpanded configuration. In another non-limiting configuration, the expandable metal frame includes one or more rows formed of vertically extending vertically extending axial longitudinal members, angular articulating members, articulating joints and form a joint between two angular articulating members, and base joints that form a connection between an angular articulating member and a vertically extending vertically extending axial longitudinal member; said each of the angular articulating joints have an inner radius at least 0.12 mm (e.g., 0.12-0.3 mm and all values and ranges therebetween) when the expandable frame is a crimped orientation; and wherein a maximum width of each of the angular articulating joints is at least 0.2 mm (e.g., 0.2-0.8 mm and all values and ranges therebetween); and wherein a maximum cross-sectional area of each of the angular articulating joints is at least 0.03 mm². (e.g., 0.03-0.5 mm² and all values and ranges therebetween).

In accordance with another and/or alternative non-limiting aspect of the present disclosure, the prosthetic heart valve includes a) an expandable frame that is balloon expandable, mechanically expandable or self-expanding, and b) a least 1 leaflet (e.g., 1-6 leaflets and all values and ranges therebetween). The one or more leaflets are supported by the expandable frame. The prosthetic heart valve can include an inner and/or outer skirt. The expandable frame optionally has multiple frame cells organized in rows or columns arranged in a cylindrical shape with a proximal and distal end. The metal frame is optionally formed of a metal alloy that plastically deforms and/or elastically deforms to enable the expandable frame to be expanded and compressed (crimped) to different geometrical states.

In accordance with another and/or alternative non-limiting aspect of the present disclosure, the frame has an open cell configuration wherein the cells of the expandable metal frame comprise vertically extending axial longitudinal members and angular articulating members, wherein the angular articulating members are connected to each other through articulating joints, and wherein the vertically extending axial longitudinal members are connected to angular articulating members through a base joint. In one non-limiting embodiment, the expandable frame has no more than 20% longitudinal foreshortening (e.g., 0-20% longitudinal foreshortening and all values and ranges therebetween) along a longitudinal axis of the expandable frame when the expandable frame is plastically deformed (e.g., expanded from the crimped to the uncrimped position), and typically no more than 5% longitudinal foreshortening; and the frame cells are comprised of at least two vertically extending axial longitudinal members, and at least two angular articulating member pairs; and wherein each angular articulating member pair includes at least two angular articulating members connected by an articulating joint; and wherein during expansion or compression of the expandable frame, the overall longitudinal length of the frame cells do not exceed the height of the vertically extending axial longitudinal members. In one non-limiting configuration, the expandable frame is formed of multiple or all non-foreshortening frame cells. In another non-limiting configuration, the angular articulating members in all cells in a row of frame cells and/or in a column of frame cells have the same length. In another non-limiting configuration, the length of the angular articulating members is measured from peak to peak of joints defining the ends of an angular articulating member, and wherein the sum of the length of the angular articulating members is less than or equal to the sum of the length of the vertically extending axial longitudinal members. In another non-limiting configuration, the geometry of the angular articulating member has independent curvature on its width through at least a portion of the length of the angular articulating member. In another non-limiting embodiment, the complete frame does not foreshorten during the expansion and/or crimping of the expandable frame even when one or more of the frame cells foreshorten during the expansion and/or crimping of the expandable frame. In another non-limiting configuration, the longitudinal length between the proximal end of the expandable frame and the distal end of the expandable frame is mostly constant or constant during changes in diameter of the expandable frame. In another non-limiting configuration, one or more longitudinal posts extend through the complete distance from the distal end to the proximal end of the expandable frame. In another non-limiting configuration, the expandable frame includes at least one vertically extending axial longitudinal member extending from the distal end of the frame to the proximal end of the frame, and a commissural attachment area is located between the proximal and distal ends of the expandable frame, and wherein the commissural attachment area forms a portion or all of an vertically extending axial longitudinal member. In another non-limiting configuration, a longitudinal distance from the commissural attachment area to the proximal end of the frame is predominantly constant during expansion and/or crimping of the expandable frame. In another non-limiting configuration, the rows and columns of the frame cells of the expandable frame are made of frame cells of equal width or equal height as adjacent circumferential or axial cells. In another non-limiting configuration, the difference in cross-sectional area of a window area of one or more frame cells of the expandable frame do not differ by more than 20% when compared to other cells in the same row of frame cell, and/or in the same column cells in in the expandable frame. In another non-limiting configuration, the expandable frame is made of even or odd numbers of cells per row. In another non-limiting configuration, the expandable frame includes an odd numbers of cells. In another non-limiting configuration, one or more or all of the rows of cells of the expandable frame are formed of 6-12 cells (and all values and ranges therebetween). In another non-limiting configuration, the prosthetic heart valve is inserted in the heart such that when the expandable frame is expanded, one or more of the frame cells will partially or fully be positioned across the access to a coronary artery.

In accordance with another and/or alternative non-limiting aspect of the present disclosure, the material used to form the expandable metal frame contains little or no nickel. Nickel and cobalt are commonly used alloys in the frames of commercial prosthetic heart valves, even though such materials have exhibited suboptimal results in terms of biocompatibility. In one non-limiting embodiments, the metal alloy used to form the expandable frame includes only trace amounts (e.g., less than 0.1 wt. %) of cobalt, chromium, and/or nickel. In another non-limiting configuration, the metal alloy used to form the expandable frame is completely absent nickel, chromium and/or cobalt.

In accordance with another and/or alternative non-limiting aspect of the present disclosure, the material used to form the expandable metal has a modulus of elasticity of at least 52000 Ksi and/or a recoil of no more than 5% when the frame is plastically deformed (e.g., expanded from the crimped to the expanded state, crimped, etc.) and no further expansion force is being applied to the expanded frame (e.g., the delivery balloon has been deflated and applies no load on the expanded frame).

In accordance with another and/or alternative non-limiting aspect of the present disclosure, the expandable frame can include distinguishing features which allows for rotational alignment of the expandable frame with the commissures of the native heart valve. In one non-limiting embodiment, the one or more distinguishing feature on the expandable heart valve are asymmetrical for identification of rotational alignment of the expandable frame. In another non-limiting embodiment, the one or more distinguishing features are attached directly to the commissure of the prosthetic heart valve frame. In another non-limiting embodiment, the one or more the distinguishing feature can be formed of radiopaque material which allows for high visibility during the insertion procedure of the prosthetic heart valve in the heart. In another non-limiting embodiment, the one or more distinguishing feature are at least partially made from a material with a density of at least 10 mg/cm³. The one or more distinguishing feature can be formed of the same or different material from the main body of the frame.

In accordance with another and/or alternative non-limiting aspect of the present disclosure, the expandable frame of the prosthetic heart valve can be optionally coated with a polymer material (e.g., silicone, PTFE, ePTFE, polyurethane, polyolefins, hydrogels, biological materials (e.g., pericardium or biological polymers such as collagen, gelatin, or hyaluronic acid derivatives), etc.). The coating can be used to partially or fully encapsulate the angular articulating members and/or vertically extending vertically extending axial longitudinal members on the frame and/or to fill-in the openings between the angular articulating members and/or vertically extending vertically extending axial longitudinal members on the frame.

In accordance with another and/or alternative non-limiting aspect of the present disclosure, there is provided a prosthetic heart valve that optionally includes an outer skirt, and wherein the outer skirt can be formed of a variety of materials (e.g., polymer [e.g., polyethylene terephthalate (PET), polyester, nylon, Kevlar®, silicon, etc.], composite material, metal, fabric material, etc.). In one non-limiting embodiment, the material used to partially or fully form the outer skirt can optionally be substantially non-elastic (i.e., substantially non-stretchable and non-compressible). In another non-limiting embodiment, the material used to partially or fully form the outer skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The outer skirt can optionally be formed from a combination of a cloth or fabric material that is coated with a flexible material or with a stretchable and/or compressible material so as to provide additional structural integrity to the outer skirt. The outer skirt generally is positioned completely around a portion of the outer surface of the frame. Generally, the outer skirt is positioned about the lower outer surface portion of the frame, but does not fully cover the upper outer surface. The size, configuration, and thickness of the outer skirt is non-limiting (e.g., thickness of 0.1-20 mils and all values and ranges therebetween). The outer skirt can be secured to the outside of the frame and/or an optional inner skirt using various means (e.g., sutures, clamp arrangement, stitches, etc.). In another non-limiting embodiment, the outer skirt can be made out of a woven material; however, non-woven materials can also or alternatively be used. In another non-limiting embodiment, the outer skirt (when used) can be used to 1) at least partially seal and/or prevent perivalvular leakage, 2) at least partially secure the leaflet structure to the frame, 3) at least partially protect the leaflets from damage during the crimping and/or expansion process, and/or 4) at least partially protect the leaflets from damage during the operation of the prosthetic heart valve in the heart.

In accordance with another and/or alternative non-limiting aspect of the present disclosure, there is provided a prosthetic heart valve that optionally includes an inner skirt that is positioned at least partially about the interior region of the frame. The inner skirt generally is positioned completely around a portion of the inner surface of the frame. Generally, the inner skirt is positioned about the lower inner surface portion of the frame, but does not fully cover the upper inner surface of the frame; however, this is not required. The inner skirt can be connected to the frame and/or the optional outer skirt and/or the one or more leaflets by a variety of arrangements (e.g., sutures, adhesive, melted connection, clamping arrangement, stitches, etc.). At least a portion of the inner skirt can optionally be located on the outer surface of the frame. Generally, the inner skirt is formed of a less flexible and/or compressible material than the optional outer skirt; however, this is not required. The inner skirt can be formed of a variety of materials (e.g., polymer [e.g., polyethylene terephthalate (PET), polyester, nylon, Kevlar®, silicon, etc.], composite material, metal, fabric material, etc.). In one non-limiting embodiment, the material used to partially or fully form the inner skirt can optionally be substantially non-elastic (i.e., substantially non-stretchable and non-compressible). In another non-limiting embodiment, the material used to partially or fully form the inner skirt can optionally be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with the stretchable and/or compressible material to provide additional structural integrity to the inner skirt. In another non-limiting embodiment, the inner skirt can be made out of a woven material; however, non-woven materials can also or alternatively be used. The size, configuration, and thickness of the inner skirt is non-limiting. The thickness of the inner skirt is generally 0.1-20 mils (and all values and ranges therebetween). Generally, the configuration of the inner skirt and outer skirt are generally different, and can optionally be formed of different materials.

In accordance with another and/or alternative non-limiting aspect of the present disclosure, the prosthetic heart valve includes a leaflet structure that be can be attached to the frame and/or the inner skirt and/or the outer skirt. The connection arrangement used to secure the leaflet structures to the frame and/or skirt is non-limiting (e.g., sutures, melted bold, adhesive, clamp arrangement, stitches, etc.). The material used to form the leaflet structures include polymers, bovine pericardial tissue, bovine tissue, porcine tissue, biocompatible synthetic materials, or various other suitable natural or synthetic materials. The tissue used to form the one or more leaflets can optionally be treated/stabilized through a method of collagen cross linking and thereafter stored dry or wet (e.g., tissue is stored dry after a glycerin-based dehydration process, etc.). In one non-limiting embodiment, the leaflet structure comprised of two or more leaflets (e.g., 2, 3, 4, 5, 6, etc.). In one non-limiting arrangement, the leaflet structure includes three leaflets arranged to collapse in a tricuspid arrangement. The configuration of the leaflet structures is non-limiting. In another non-limiting embodiment, the leaflets of the leaflet structure can optionally be secured to one another at their adjacent sides to form commissures of the leaflet structure (the edges where the leaflets come together). The leaflet structure can be secured together by a variety of connection arrangement (e.g., sutures, adhesive, melted bond, clamping arrangement, stitches, etc.). In another non-limiting embodiment, one or more of the leaflets can optionally include reinforcing structures or strips to 1) facilitate in securing the leaflets together, 2) facilitate in securing the leaflets to the skirt and/or frame, and/or 3) inhibit or prevent tearing or other types of damage to the leaflets.

In accordance with another and/or alternative non-limiting aspect of the present disclosure, there is provided a prosthetic heart valve that is configured to be inserted into a desired location in the body (e.g., the aortic valve, tricuspid valve, pulmonary valve, mitral valve). The frame of the prosthetic heart valve can be at least partially formed of a plastically-expandable material that permits crimping of the frame to a smaller profile for delivery and expansion of the prosthetic heart valve to a larger profile. The expansion of the crimped frame can be optionally be use of an expansion device such as, but not limited to, a balloon of on a balloon catheter.

In accordance with another and/or alternative non-limiting aspect of the present disclosure, the use of the metal alloy to partially or fully form the frame of the prosthetic heart valve can be used to increase the strength and/or hardness and/or durability of the frame of the prosthetic heart valve as compared with stainless steel or chromium-cobalt alloys or titanium alloys; thus, less quantity of metal alloy can be used in the frame of the prosthetic heart valve to achieve similar strengths as compared to frames of prosthetic heart valves formed of different metals. As such, the resulting prosthetic heart valve can be made smaller and less bulky by using the metal alloy without sacrificing the strength and durability of the prosthetic heart valve. Such a prosthetic heart valve can have a smaller profile, thus can be inserted in smaller areas, openings, and/or passageways. The metal alloy can also increase the radial strength of the frame of the prosthetic heart valve. For instance, the thickness of the angular articulating members and/or vertically extending vertically extending axial longitudinal members of the frame of the prosthetic heart valve and/or the wires used to at least partially form the frame of the prosthetic heart valve can be made thinner and achieve a similar or improved radial strength as compared with thicker walled frames of prosthetic heart valves formed of stainless steel, titanium alloys, or cobalt and chromium alloys. The metal alloy can also improve stress-strain properties, bendability and flexibility of the frame of the prosthetic heart valve, thus increasing the life of the prosthetic heart valve. For instance, the prosthetic heart valve can be used in regions that subject the prosthetic heart valve to bending. Due to the improved physical properties of the prosthetic heart valve from the metal alloy, the prosthetic heart valve has improved resistance to fracturing in such frequent bending environments. In addition or alternatively, the improved bendability and flexibility of the frame of the prosthetic heart valve due to the use of the metal alloy can enable the prosthetic heart valve to be more easily inserted into various regions of a body. The metal alloy can also reduce the degree of recoil during the crimping and/or expansion of the frame of the prosthetic heart valve. For example, the prosthetic heart valve better maintains its crimped form and/or better maintains its expanded form after expansion due to the use of the metal alloy. As such, when the prosthetic heart valve is to be mounted onto a delivery device when the prosthetic heart valve is crimped, the prosthetic heart valve better maintains its smaller profile during the insertion of the prosthetic heart valve into various regions of a body. Also, the prosthetic heart valve better maintains its expanded profile after expansion so as to facilitate in the success of the prosthetic heart valve in the treatment area. In addition to the improved physical properties of the prosthetic heart valve by use of the metal alloy, the metal alloy can optionally have improved radiopaque properties as compared to standard materials such as stainless steel or cobalt-chromium alloy, thus reducing or eliminating the need for using marker materials on the prosthetic heart valve.

In accordance with another and/or alternative non-limiting aspect of the present disclosure, the use of the metal alloy to form all or a portion of the prosthetic heart valve can result in several advantages over prosthetic heart valves formed from other materials. These advantages include, but are not limited to:

The metal alloy used to partially or fully form the frame of the prosthetic heart valve optionally has increased strength and/or hardness as compared with stainless steel, chromium-cobalt alloys, or titanium alloys, thus a less quantity of metal alloy can be used in the prosthetic heart valve to achieve similar strengths as compared to prosthetic heart valves formed of different metals. As such, the resulting prosthetic heart valve can be made smaller and less bulky by using the metal alloy without sacrificing the strength and durability of the prosthetic heart valve. The prosthetic heart valve can also have a smaller profile, thus can be inserted into smaller areas, openings, and/or passageways. The thinner angular articulating members and/or vertically extending vertically extending axial longitudinal members of metal alloy to form the frame or other portions of the prosthetic heart valve can be used to form a frame or other portion of the prosthetic heart valve having a strength that would require thicker angular articulating members and/or vertically extending vertically extending axial longitudinal members or other structures of the prosthetic heart valve when formed by stainless steel, chromium-cobalt alloys, or titanium alloys.

The increased strength of the metal alloy used in the frame of the prosthetic heart valve optionally results in the increased radial strength of the prosthetic heart valve. For instance, the thickness of the walls of the prosthetic heart valve can optionally be made thinner and achieve a similar or improved radial strength as compared with thicker walled prosthetic heart valves formed of stainless steel, cobalt and chromium alloy, or titanium alloy.

The metal alloy used to partially or fully form the frame of the prosthetic heart valve optionally has a reduced degree of recoil during the crimping and/or expansion of the prosthetic heart valve compared with stainless steel, chromium-cobalt alloys, or titanium alloys. The prosthetic heart valve formed of the metal alloy better maintains its crimped form and/or better maintains its expanded form after expansion due to the use of the metal alloy. As such, when the prosthetic heart valve is to be mounted onto a delivery device when the prosthetic heart valve is crimped, the prosthetic heart valve better maintains its smaller profile during the insertion of the prosthetic heart valve in a body passageway. Also, the prosthetic heart valve better maintains its expanded profile after expansion to facilitate in the success of the prosthetic heart valve in the treatment area.

The use of the metal alloy in the frame of the prosthetic heart valve optionally results in the prosthetic heart valve better conforming to an irregularly shaped body passageway when expanded in the body passageway compared to a prosthetic heart valve formed by stainless steel, chromium-cobalt alloys, or titanium alloys.

The metal alloy used to partially or fully form the frame of the prosthetic heart valve optionally has improved radiopaque properties compared to standard materials such as stainless steel or cobalt-chromium alloy, thus reducing or eliminating the need for using marker materials on the prosthetic heart valve. For example, the metal alloy is at least about 10-20% more radiopaque than stainless steel or cobalt-chromium alloy.

The metal alloy used to partially or fully form the frame of the prosthetic heart valve optionally has improved fatigue ductility when subjected to cold-working compared to the cold-working of stainless steel, chromium-cobalt alloys, or titanium alloys.

The metal alloy used to partially or fully form the frame of the prosthetic heart valve optionally has improved durability compared to stainless steel, chromium-cobalt alloys, or titanium alloys.

The metal alloy used to partially or fully form the frame of the prosthetic heart valve optionally has improved hydrophilicity compared to stainless steel, chromium-cobalt alloys, or titanium alloys.

The metal alloy used to partially or fully form the frame of the prosthetic heart valve optionally has reduced ion release in the body passageway compared to stainless steel, chromium-cobalt alloys, or titanium alloys.

The metal alloy used to partially or fully form the frame of the prosthetic heart valve optionally is less of an irritant to the body than stainless steel, cobalt-chromium alloy, or titanium alloys, thus can result in reduced inflammation, faster healing, increased success rates of the prosthetic heart valve. When the prosthetic heart valve is expanded in a body passageway, some minor damage to the interior of the passageway can occur. When the body begins to heal such minor damage, the body has less adverse reaction to the presence of the metal alloy compared to other metals such as stainless steel, cobalt-chromium alloy, or titanium alloy.

The metal alloy used to partially or fully form the frame of the prosthetic heart valve optionally has a magnetic susceptibility that is lower that CoCr alloy, TiAlV alloys, and/or stainless steel, thus resulting in less incidence of potential defects to the prosthetic heart valve or complications to the patent after implantation of the prosthetic heart valve when the patient is subjected to an MRI or other prosthetic heart valve that generates a strong magnetic field.

In accordance with another and/or alternative non-limiting aspect of the present disclosure, the frame of the prosthetic heart valve has one or features that include, but are not limited to:
High Radial Strength.
Small or Low Crimp Profile.
Reduce Recoil when expanded and/or crimped.
Little or No Longitudinal Foreshortening when expanded.
Smooth curvature at peaks and along angular articulating members.
Symmetrical Design for restoration of valve function and visualization of frame.
Physical Markers on frame for commissural alignment.
Open cell design.
Open cell aligned with Coronary for hemodynamic and reintervention.

In accordance with another and/or alternative non-limiting aspect of the present disclosure, the prosthetic heart valve includes a radially collapsible and expandable frame that includes a plurality of vertically extending vertically extending axial longitudinal members. In one non-limiting embodiment, one or more of the vertically extending vertically extending axial longitudinal members extends 80-100% (and all values and ranges therebetween) of the longitudinal length of the frame. In one non-limiting configuration, one or more of the vertically extending vertically extending axial longitudinal members can be formed by a single longitudinal shaft or plurality of longitudinal segments that are connected together and extend along the same central longitudinal axes at least 80% (e.g., 80-100% and all values and ranges therebetween) of the longitudinal length of the frame. When one or more of the vertically extending vertically extending axial longitudinal members are formed by or plurality of longitudinal segments, each of the longitudinal segments extends 10-70% (and all values and ranges therebetween) of the longitudinal length of the frame, and wherein the longitudinal length of each of the vertically extending vertically extending axial longitudinal members can be the same or different. When one or more of the vertically extending vertically extending axial longitudinal members are formed by or plurality of longitudinal segments, each of the longitudinal segments are stacked and connected together along the longitudinal length of the frame, and typically are all aligned along the same central longitudinal axis of the stacked longitudinal segments. As such, one or more vertically extending vertically extending axial longitudinal members can be formed form a) stacked longitudinal segments that are connected together along the longitudinal length of the frame, and wherein the stacked longitudinal segments that are optionally connected together (e.g., weld, solder, adhesive, melted connection, etc.) along same central longitudinal axis of the stacked longitudinal segments, or b) formed of a single longitudinal component formed of a single piece of material (e.g., a frame cut from a single piece of material to form the vertically extending vertically extending axial longitudinal members, the angular articulating members, and any of features of the cut frame will result in these frame features being formed of a single piece of material), and wherein the single longitudinal component optionally extends along a single longitudinal axis. In another non-limiting embodiment, the vertically extending vertically extending axial longitudinal members are configured to limit or eliminate longitudinal foreshortening of the frame when the frame is plastically deformed (e.g., expanded from the crimped to the expanded position). When the frame includes a plurality of vertically extending vertically extending axial longitudinal members that have a longitudinal length of 80-100% of the longitudinal length of the frame and are spaced at various location about the about the perimeter of the frame, the vertically extending vertically extending axial longitudinal members facilitate in inhibiting or preventing longitudinal foreshortening of the frame when expanded.

In another non-limiting embodiment, thickness, width, and/or cross-sectional area of the vertically extending vertically extending axial longitudinal members along the longitudinal axis of the vertically extending vertically extending axial longitudinal members can be constant or vary. In one non-limiting configuration, the thickness, width, and/or cross-sectional area of each of the vertically extending vertically extending axial longitudinal members along the longitudinal axis of the vertically extending vertically extending axial longitudinal members varies. In another non-limiting configuration, a portion of the one or more of the vertically extending vertically extending axial longitudinal members that is located closer to a top portion of the frame has a thickness, width, and/or cross-sectional area that is less than a thickness, width and/or cross-sectional area of a portion of one or more of the vertically extending vertically extending axial longitudinal members that are located closer to the bottom portion of the frame.

In accordance with another and/or alternative non-limiting aspect of the present disclosure, the prosthetic heart valve includes a radially collapsible and expandable frame that includes a plurality of the angular articulating members. In one non-limiting embodiment, the frame includes at least two rows (e.g., 2-12 and all values and ranges therebetween) of angular articulating members. In another non-limiting embodiment, the shape, size, and/or configuration of a plurality or a majority or all of the angular articulating members on each row of angular articulating members are the same. In one non-limiting configuration, the shape, size, and configuration of all of the angular articulating members on one or more rows of angular articulating members are the same. In another non-limiting configuration, the shape, size, and configuration of some of the angular articulating members on a row of angular articulating members are different from other angular articulating members on the same row of angular articulating members.

In accordance with another and/or alternative non-limiting aspect of the present disclosure, the prosthetic heart valve includes a radially collapsible and expandable frame that includes a plurality of the angular articulating members wherein a plurality, a majority or all of the angular articulating members are formed of a centrally located arcuate portion or semi-circular portion (i.e., articulating joint, etc.), and first and second arms that extend from each side of the semi-circular portion. In one non-limiting embodiment, each of the first and second arms include one or more undulations. In another non-limiting embodiment, the longitudinal length of one or both arms is greater than a width of the semi-circular portion. In another non-limiting configuration, the combined longitudinal length of the two arms constitutes at least 60% (e.g., 60-95% and all values and ranges therebetween) of the total longitudinal length of the angular articulating members. In another non-limiting embodiment, a plurality of the angular articulating members has first and second arms that are the same length, size, shape, and/or configuration. In one non-limiting configuration, a plurality of the angular articulating members has first and second arms that are not the same length, size, shape, and/or configuration. In another non-limiting configuration, the semi-circular portion has an arc length of 60-190° (and all values and ranges therebetween) when the frame is in the expanded orientation, and the semi-circular portion has an arc length of 80-340° (and all values and ranges therebetween) when the frame is in the crimped or collapsed orientation.

In accordance with another and/or alternative non-limiting aspect of the present disclosure, the prosthetic heart valve includes a radially collapsible and expandable frame that includes three or more rows of the angular articulating members and wherein the spacing of the angular articulating members between adjacently positioned rows of angular articulating members can be the same or different.

In accordance with another and/or alternative non-limiting aspect of the present disclosure, the prosthetic heart valve includes a radially collapsible and expandable frame that includes one or more vertically extending vertically extending axial longitudinal members that have one or more frame opening arrangements that can optionally be used as securing locations for the one of more leaflet structures, leaflet, inner skirt, and/or outer skirt. In one non-limiting configuration, only 2-4 of the vertically extending vertically extending axial longitudinal members include the one or more frame opening arrangements. In another non-limiting configuration, the one or more frame opening arrangement are located one a top region of the vertically extending vertically extending axial longitudinal member. In one non-limiting embodiment, one or more of the frame opening arrangements includes a first and optionally a second frame opening. The size and shape of the first and optional second frame opening are non-limiting. The one or more of the frame opening arrangements can optionally be used as a marker to facilitate in the proper positioning of the frame and prosthetic heart valve in the heart (e.g., proper commissural alignment of the prosthetic heart valve in the heart valve region).

In accordance with another and/or alternative non-limiting aspect of the present disclosure, one or more commissural alignment features can optionally be connected to or formed on the top end or bottom end of one or more vertically extending vertically extending axial longitudinal members. The shape and size of the one or more commissural alignment features is non-limiting. The use of the one or more commissural alignment features can be used to facilitate in the proper positioning of the frame and prosthetic heart valve in the heart (e.g., proper commissural alignment of the prosthetic heart valve in the heart valve region). In one non-limiting configuration, when one or more of the vertically extending vertically extending axial longitudinal members include one or more of the frame opening arrangements, such vertically extending vertically extending axial longitudinal members can also optionally include one or more of the commissural alignment features, and optionally located at the top of the vertically extending vertically extending axial longitudinal member.

In accordance with another and/or alternative non-limiting aspect of the present disclosure, the prosthetic heart valve includes a radially collapsible and expandable frame that includes one or more of the following features: a) high radial strength after expansion of the frame, b) small crimp profile; c) use of a material that minimizes recoil after expansion of the frame; d) little or no longitudinal foreshortening of the frame during expansion; e) smooth curvature at peaks and along angular articulating members and/or the vertically extending vertically extending axial longitudinal members of the frame; f) symmetrical design for restoration of valve function and visualization of frame; g) markers on frame for commissural alignment; and/or h) open cells that can be aligned with coronary for hemodynamic and reintervention.

One non-limiting object of the present disclosure is the provision of the refractory metal alloy or a metal alloy that includes at least 5 awt. % (e.g., 5-99 awt. % and all values and ranges therebetween) rhenium and/or hafnium that can be used to partially or fully form a frame of a prosthetic heart valve.

Another and/or alternative non-limiting object of the present disclosure is the provision of a prosthetic heart valve that includes a frame that is partially or fully formed of refractory metal alloy or a metal alloy that includes at least 5 awt. % (e.g., 5-99 awt. % and all values and ranges therebetween) rhenium and/or hafnium and which prosthetic heart valve has improved procedural success rates.

Another and/or alternative non-limiting object of the present disclosure is the provision of a prosthetic heart valve that includes a frame has a geometry in combination with the frame being partially or fully formed of refractory metal alloy or a metal alloy that includes at least 5 awt. % (e.g., 5-99 awt. % and all values and ranges therebetween) rhenium and/or hafnium to enable the formation of a frame a) that has an open cell geometry in the frame of the prosthetic heart valve that can be used to reduce delivery system size reducing vascular and neurological complications, b) that has high radial strength using an open cell pattern due to the high yield strength and ultimate tensile strength of the metal alloy, c) that has improved restoration of the physiologic EOA in challenging, heavily calcified valves that exert high force on the bioprosthetic valve, while allowing a reduced crimp diameter for vascular access, d) that has improved restoration of the physiologic EOA that results in greater longevity of the bioprosthetic valve, e) that includes a material having lower recoil than the traditional materials of stainless steel, chromium-cobalt, or titanium alloys resulting in less recoil of the frame when expanded which leads to decreased risk of valve embolization, decreased paravalvular leak due to improved conformability of the native anatomy, more accurate restoration of the physiologic EOA, and decrease conduction system injury due to a lower balloon inflation diameter required to obtain the physiologic EOA after balloon inflation, f) that has an open cell geometry that allows for a frame geometry with no longitudinal foreshortening, which allows for more accurate placement of the valve in the native annulus, since a frame with no longitudinal foreshortening has a shorter initial frame length allowing for a shorter expansion balloon on the catheter, which can decrease conduction system injury, g) that has an open cell geometry with commissural alignment markers and an open cell between the commissures that allows for proper placement of the bioprosthetic valve in relation to the native commissures of the valve for proper hemodynamic function in regard to wash out of the valve and blood flow to the coronaries, which leads to better durability and longevity of the valve, and access and re-intervention of the coronaries preventing future adverse events, h) that has open cell geometry with radial symmetry, longitudinal symmetry, and little or no longitudinal foreshortening allows for symmetrical and cylindrical expansion of the prosthetic valve resulting in lower rates of leaflet thrombosis and structural valve deterioration, i) that is formed of a metal alloy with no nickel content so as to inhibit or prevent allergic response due to the presence of nickel and restenosis associated with nickel content, j) that is formed of a metal alloy that has reduced ion release after the frame is implanted in a patient, k) that is formed of a metal alloy that has improve hydrophilicity, l) that is partially or fully coated with an enhancement coating to improve one or more properties of the frame and/or to increase the life of the prosthetic heart valve, and/or m) that has angular articulating members having a certain configuration that enhances the performance of the frame during the expansion of the frame.

Another and/or alternative non-limiting object of the present disclosure is the provision of a prosthetic heart valve for implantation into a heart; the prosthetic heart valve includes an expandable metal frame, a leaflet structure supported by the expandable metal frame, and an outer skirt secured to the expandable metal frame; the expandable metal frame is configured to expand from a crimped orientation to an expanded orientation when the prosthetic heart valve is positioned in a treatment site in the heart; the expandable metal frame includes a plurality of angular articulating members and a plurality of vertically extending vertically extending axial longitudinal members; the angular articulating members and the vertically extending vertically extending axial longitudinal members are connected together to form a plurality of cells in the expandable metal frame organized into rows whereby the rows of cells are connected by vertically extending vertically extending axial longitudinal members; one or more of the vertically extending vertically extending axial longitudinal members have a longitudinal length that is at least 10% a longitudinal length of the expandable metal frame when the expandable metal frame is in the expanded orientation.

Another and/or alternative non-limiting object of the present disclosure is the provision of a prosthetic heart valve for implantation into a heart wherein the leaflet structure comprises a plurality of leaflets, each of the leaflets has an upper edge portion, a lower edge portion and two side flaps, wherein each side flap is connected to an adjacent side flap of another leaflet, at least a portion of the leaflet structure connected to the expandable metal frame and/or the inner skirt and/or the outer skirt; and wherein the leaflet structure is optionally attached to the expandable metal frame and/or the inner skirt and/or the outer skirt using a plurality of sutures, stitches, staples or adhesive.

Another and/or alternative non-limiting object of the present disclosure is the provision of a prosthetic heart valve for implantation into a heart that includes an expandable metal frame and at least one leaflet that is connected to the expandable metal frame; the expandable metal frame is configured to be crimped and further expanded from a crimped orientation to an expanded orientation opening in a body passageway; the expandable metal frame has distal and proximal ends; the expandable metal frame a) has an open cell configuration that includes a plurality of frame cells and wherein the open cell configuration has high radial strength, b) is formed of a material that has reduced recoil thus resulting in reduced recoil of the frame when expanded, and c) can be crimped a small diameter; the expandable metal frame has one or more of the following properties: i) has a yield strength of at least 110 Ksi, ii) has a modulus of elasticity of at least 52000 Ksi, iii) has a frame geometry that has a maximum of 9 frame cells per horizontal row, wherein horizontal is defined as perpendicular to a longitudinal axis of the expandable metal frame, iv) that is optionally at least partially formed of refractory metal alloy or a rhenium and/or hafnium containing metal alloy that includes at least 5 awt. % rhenium and/or rhenium, v) is formed of material that has a reduced recoil when bent such that the expandable frame has no more than 5% recoil when the expandable metal frame is plastically deformed, vi) has longitudinal foreshortening of no more than 20% (e.g., 0-20% and all values and ranges therebetween) when the expandable metal frame is plastically deformed (e.g., the longitudinal length of the expanded frame reduces in longitudinal length of 0-20% and all values and ranges therebetween when the expandable frame is crimped or when the expandable frame is expanded from the crimped positioned to the expanded position), and/or vii) that is at least partially formed of a metal alloy that has an elongation of at least 20%.

Another and/or alternative non-limiting object of the present disclosure is the provision of a prosthetic heart valve for implantation into a heart that wherein one or more of the frame cells each include at least two vertically extending vertically extending axial longitudinal members, and at least two angular articulating member pairs; each of the angular articulating member pairs includes at least two angular articulating members connected by an articulating joint; at least one of the vertically extending vertically extending axial longitudinal members extends from the distal end to the proximal end of the expandable frame; and wherein during expansion and/or crimping of the expandable frame, an overall longitudinal length of the frame cell does not exceed a longitudinal length of the vertically extending vertically extending axial longitudinal members, and/or the frame foreshortens no more than 10% (e.g., 0-10% and all values and ranges therebetween foreshortening).

Another and/or alternative non-limiting object of the present disclosure is the provision of a prosthetic heart valve comprising an expandable metal frame and at least one leaflet that is connected to the expandable metal frame; the expandable metal frame is configured to be crimped and further expand from a crimped orientation to an expanded orientation opening in a body passageway; the expandable metal frame has distal and proximal ends; the expandable metal frame includes a) an open cell configuration that includes a plurality of frame cells and at least two rows of frame cells, and/or b) is formed of a material that has a recoil of less than 6% thus resulting in reduced recoil of the expandable metal frame when expanded from the crimped orientation to the expanded orientation.

Another and/or alternative non-limiting object of the present disclosure is the provision of a prosthetic heart valve wherein one or more of the frame cells includes at least two vertically extending vertically extending axial longitudinal members and at least two angular articulating members; each of the angular articulating members includes first and second arms that are connected to an articulating joint; the an articulating joint having an arcuate shape or semi-circular portion; and wherein each of the vertically extending vertically extending axial longitudinal members has a continuous linear shape of at least 90% of a longitudinal length of the vertically extending vertically extending axial longitudinal members . . .

Another and/or alternative non-limiting object of the present disclosure is the provision of a prosthetic heart valve wherein at least one vertically extending vertically extending axial longitudinal member in the row of frame cells is aligned along a same longitudinal axis to form an aligned group of vertically extending vertically extending axial longitudinal members and the aligned group of vertically extending vertically extending axial longitudinal members fully extends from the distal end to the proximal end of the expandable metal frame; and wherein during expansion and/or crimping of the expandable metal frame an overall longitudinal length of each of the frame cells in a row of frame cells does not exceed the longitudinal length of each of the vertically extending axial longitudinal members in the frame cell, and/or the frame foreshortens no more than 10% (e.g., 0-10% and all values and ranges therebetween foreshortening).

Another and/or alternative non-limiting object of the present disclosure is the provision of a prosthetic heart valve wherein one or more of the angular articulating members include one or more independent radii across their longitudinal length.

Another and/or alternative non-limiting object of the present disclosure is the provision of a prosthetic heart valve wherein a sum of longitudinal lengths of the angular articulating members is greater than or equal a longitudinal length of the vertically extending vertically extending axial longitudinal member.

Another and/or alternative non-limiting object of the present disclosure is the provision of a prosthetic heart valve wherein a longitudinal length of the expandable frame is equivalent to the longitudinal length of at least one of the vertically extending vertically extending axial longitudinal members during expansion and crimping of the expandable frame.

Another and/or alternative non-limiting object of the present disclosure is the provision of a prosthetic heart valve further including a commissural alignment marker that is positioned in the expandable frame; the commissural alignment marker formed of a same material as the material used to form the expandable frame.

Another and/or alternative non-limiting object of the present disclosure is the provision of a prosthetic heart valve wherein the angular articulating members in the frame cells in a same column and/or same row of frame cells are of a same longitudinal length.

Another and/or alternative non-limiting object of the present disclosure is the provision of a prosthetic heart valve wherein vertices of adjacently positioned frame cells in adjacent rows are aligned to within no more than 5% of a total longitudinal length of the angular articulating members.

Another and/or alternative non-limiting object of the present disclosure is the provision of a prosthetic heart valve wherein an area of each of a most distal row of frame cells and an area of each of a most proximal end of frame cells on the expandable frame does not differ by more than 20%.

Another and/or alternative non-limiting object of the present disclosure is the provision of a prosthetic heart valve wherein the material of the expandable frame is at least partially made of a metal alloy that includes less than 1 wt. % nickel and/or less than 0.1 wt. % cobalt.

Another and/or alternative non-limiting object of the present disclosure is the provision of a prosthetic heart valve wherein the expandable metal frame includes a plurality of angular articulating members and a plurality of vertically extending vertically extending axial longitudinal members; the angular articulating members and the vertically extending vertically extending axial longitudinal members are connected together to form a plurality of open cells in the expandable metal frame that are organized into rows; each of the cells includes at least one the vertically extending vertically extending axial longitudinal members and at least two angular articulating members; each of the angular articulating members includes a plurality of arcuate portions along a longitudinal length of the angular articulating member; one or more of the vertically extending vertically extending axial longitudinal members has a continuous linear shape of at least 80% of a longitudinal length of the vertically extending vertically extending axial longitudinal member; the expandable metal frame has a longitudinal foreshortening of no more than 20% when the expandable metal frame is plastically deformed.

Another and/or alternative non-limiting object of the present disclosure is the provision of a prosthetic heart valve wherein one or more of the vertically extending vertically extending axial longitudinal members have a longitudinal length that is 70-100% of the longitudinal length of the expandable metal frame when the expandable metal frame is in the expanded orientation.

Another and/or alternative non-limiting object of the present disclosure is the provision of a prosthetic heart valve wherein the expandable metal frame includes a first cell row, and a second cell row, each of the first and second cell rows include a plurality of angular articulating members; each of the angular articulating members in the first cell row includes first and second ends, and wherein the first end of each of the angular articulating members is connected to one of the vertically extending vertically extending axial longitudinal members and the second end of each of the angular articulating members is connected to a different vertically extending vertically extending axial longitudinal members; each of the angular articulating members in the second cell row includes first and second ends, and wherein a plurality or all of the first ends of a plurality or all of the angular articulating members is connected to one of the vertically extending vertically extending axial longitudinal members and a plurality or all of the second ends of a plurality or all of the angular articulating members is connected to a different vertically extending vertically extending axial longitudinal member.

Another and/or alternative non-limiting object of the present disclosure is the provision of a prosthetic heart valve wherein the expandable metal frame includes a first cell row, a second cell row and a third cell row, each of the first, second and third cell rows include a plurality of angular articulating members; each of the angular articulating members in the first cell row includes first and second ends, and wherein the first end of each of the angular articulating members is connected to one of the vertically extending vertically extending axial longitudinal members and the second end of each of the angular articulating members is connected to a different vertically extending vertically extending axial longitudinal members; each of the angular articulating members in the second cell row includes first and second ends, and wherein a plurality or all of the first ends of a plurality or all of the angular articulating members is connected to one of the vertically extending vertically extending axial longitudinal members and a plurality or all of the second ends of a plurality or all of the angular articulating members is connected to a different vertically extending vertically extending axial longitudinal member; and each of the angular articulating members in the third cell row includes first and second ends, and wherein a plurality or all of the first ends of a plurality or all of the angular articulating members is connected to one of the vertically extending vertically extending axial longitudinal members and a plurality or all of the second ends of a plurality or all of the angular articulating members is connected to a different vertically extending vertically extending axial longitudinal member.

Another and/or alternative non-limiting object of the present disclosure is the provision of a prosthetic heart valve wherein the expandable metal frame has a) is formed of material that has a reduced recoil when bent such that the expandable frame has no more than 5% recoil when the expandable metal frame is crimped to a crimped state, b) is formed of material that has a reduced recoil when bent such that the expandable frame has no more than 5% recoil when the expandable metal frame is expanded from a crimped state to an expanded state, and/or c) a longitudinal foreshortening of less than 20% when the expandable metal frame is expanded from the crimped state.

Another and/or alternative non-limiting object of the present disclosure is the provision of an expandable prosthetic heart valve comprising an expandable metal frame for implantation into a body passageway; said expandable metal frame is configured to be crimped and expanded from a crimped orientation to an expanded orientation in an opening in the body passageway; said expandable metal frame has distal and proximal ends; said expandable metal frame includes a plurality of frame cells and at least two rows of frame cells; said expandable metal frame has one or more of the following properties: i) at least 70-100% of said expandable metal frame is formed of a metal alloy that has a yield strength of at least 110 Ksi, ii) at least 70-100% of said expandable metal frame is formed of a metal alloy that has a modulus of elasticity of at least 35000 Ksi, iii) at least 70-100% of said expandable metal frame is formed of a metal alloy that is formed of a refractory metal alloy, or a rhenium and/or hafnium metal alloy that includes at least 5 awt. % rhenium and/or hafnium and optionally includes one or more metals selected from the group consisting of Mo, Cr, Co, Ni, Ti, Ta, Nb, Zr, and W, iv) the expandable metal frame is formed of material that has a recoil of no more than 10% recoil when said expandable metal frame is plastically deformed, v) the expandable metal frame has a longitudinal foreshortening of no more than 20% when said expandable metal frame is plastically deformed, vi) each of the cells includes at least one vertically extending vertically extending axial longitudinal member and at least two angular articulating members to form an open cell configuration, and wherein each of the angular articulating members optionally includes a plurality of arcuate portions along a longitudinal length of the angular articulating member, and wherein one or more of the vertically extending vertically extending axial longitudinal members optionally has a continuous linear shape of at least 80% of a longitudinal length of the vertically extending vertically extending axial longitudinal member; each of the angular articulating members optionally includes first and second arms that are connected to an articulating joint; wherein an articulating joint optionally has an arcuate shape or semi-circular portion; and wherein at least one vertically extending vertically extending axial longitudinal member in a row of frame cells is optionally aligned along a same longitudinal axis to form an aligned group of vertically extending vertically extending axial longitudinal members, and wherein the one or more vertically extending vertically extending axial longitudinal members fully extend from said distal end to said proximal end of said expandable metal frame; and wherein during expansion and/or crimping of the expandable metal frame an overall longitudinal length of each of said frame cells in a row of frame cells does not exceed the longitudinal length of one or more of the vertically extending vertically extending axial longitudinal members in the frame cell; and wherein one or more of the angular articulating members optionally include one or more independent radii across its longitudinal length; and wherein a sum of longitudinal lengths of the angular articulating members is optionally greater than or equal a sum of longitudinal lengths of one or more of vertically extending vertically extending axial longitudinal members; and wherein a longitudinal length of said expandable frame is optionally equivalent to said longitudinal length of at least one of the vertically extending vertically extending axial longitudinal members during expansion and crimping of said expandable frame; and wherein the expandable frame optionally includes a commissural alignment marker that is positioned in the expandable frame, and wherein the commissural alignment marker is optionally formed of a same material as the material used to form the expandable frame, and wherein the material used to form the commissural alignment marker is optionally a metal that has a density of greater than 10 mg/cm³; and wherein the angular articulating members in the frame cells in a same column and/or same row of frame cells are optionally of a same longitudinal length; and wherein the material of the expandable frame is made out of a metal alloy that includes less than 1 wt. % nickel and/or less than 0.1 wt. % cobalt; and wherein the frame is optionally coated with an enhancement layer.

These and other advantages will become apparent to those skilled in the art upon the reading and following of this description.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1A is an illustration of a TAV in accordance with the present disclosure.

FIG. 1B is a portion of a prior art catheter.

FIGS. 1C-1E illustrate a typical TAVR procedure for inserting the TAV into a valve of a heart.

FIG. 2 is an illustration of the TAV of FIG. 1A illustrating features of the vertically extending axial longitudinal members and the angular articulating members of the non-limiting frame.

FIG. 3 is a front elevation view of a non-limiting frame of a TAV in the expanded state in accordance with the present disclosure.

FIG. 4 is a front view of a flat non-limiting frame of a TAV in the expanded state in accordance with the present disclosure.

FIG. 5 is a front view of a flat non-limiting frame of a TAV in the crimped or unexpanded state in accordance with the present disclosure.

FIG. 6A is a front view of another flat non-limiting frame of a TAV in the expanded state in accordance with the present disclosure.

FIG. 6B is a front view of a flat frame in the expanded state of FIG. 6A that includes non-limiting dimensions of the non-limiting frame.

FIG. 6C-6M illustrates various features and structures of the non-limiting TAV frame.

FIG. 7 is a table that lists comparative yield strength and Modulus of various metal alloys.

FIG. 8 is a graft that compares the radial strength of frame form of a MoRe alloy to a frame formed of CoCr alloy.

FIG. 9 is a graph that illustrates the amount of recoil of several different metal alloys.

FIG. 10 is an illustration that compares the conformability of a metal strip or wire formed of refractory metal to the shape of a die surface as compared to the conformity of a metal strip or wire of CoCr alloy on the same die surface.

FIGS. 11A and 11B are illustrations that compares the conformability of a TAV frame formed of refractory metal alloy that is expanded in a non-circular aortic valve that includes calcium deposits to a similar shaped and configured TAV frame formed of CoCr alloy that is expanded in the same non-circular aortic valve, and which illustrates that the paravalvular leak (PVL) about a TAV having a frame formed of CoCr alloy is greater than the PVL about a TAV having a frame formed of refractory metal alloy due the increase conformability of the frame formed of refractory metal alloy as compared to the conformability of the frame formed of CoCr alloy.

FIGS. 12A-12C illustrate stress vs. reduction in percent area graphs of TiAIV alloy, CoCr alloy, and MoRe alloy.

FIG. 13 is a graph that illustrates the differences of stiffness and yield strength of a MoRe alloy, CoCr alloy, and TiAlV alloy.

FIGS. 14A-14C are graphs that illustrate the strength and fatigue ductility of a TiAIV alloy, CoCr alloy, and MoRe alloy.

FIG. 15 illustrates the hydrophilicity of a MoRe alloy, a CoCr alloy, and a TiAlV alloy.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

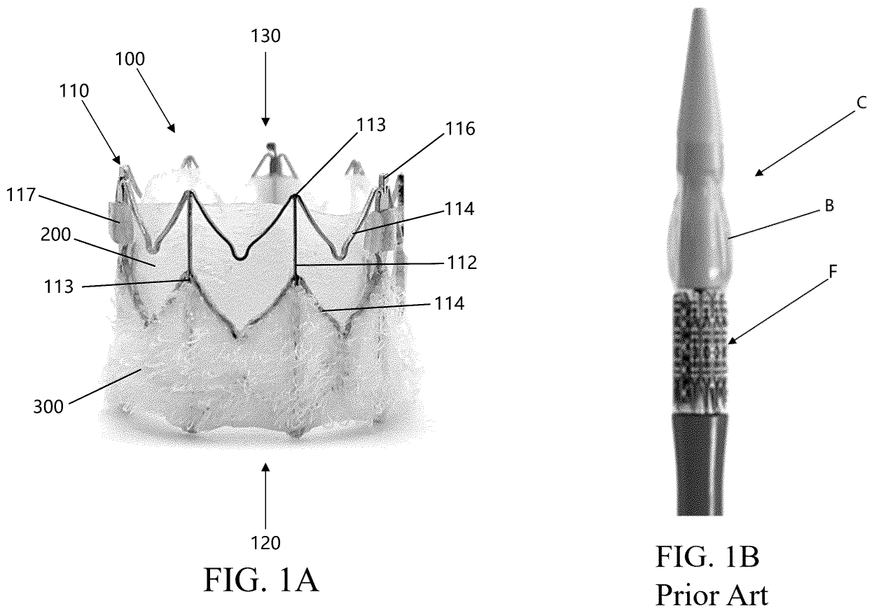

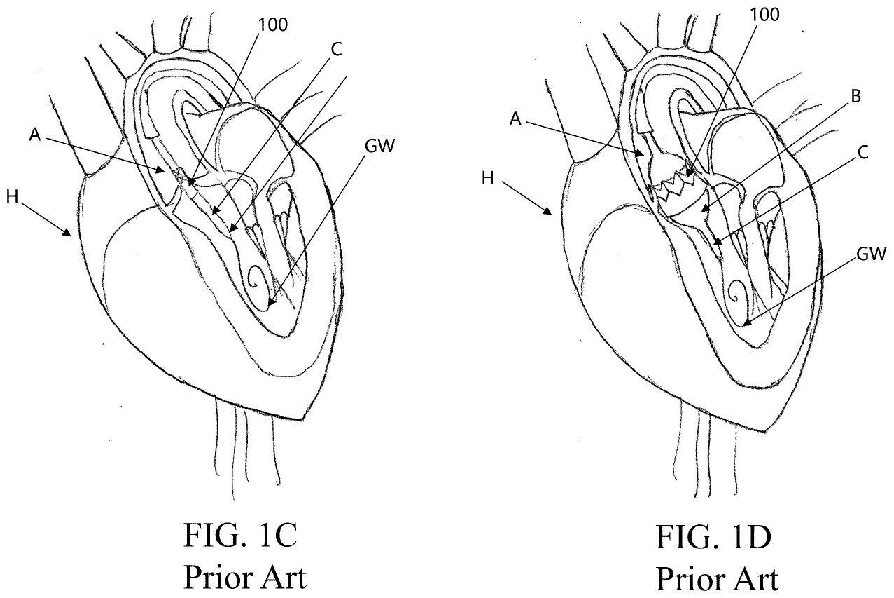

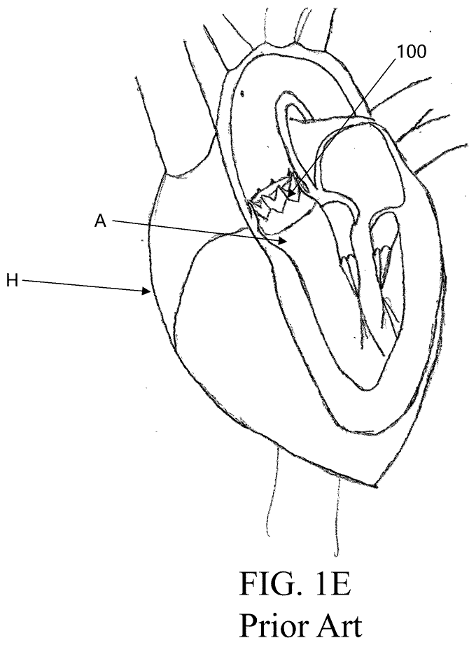

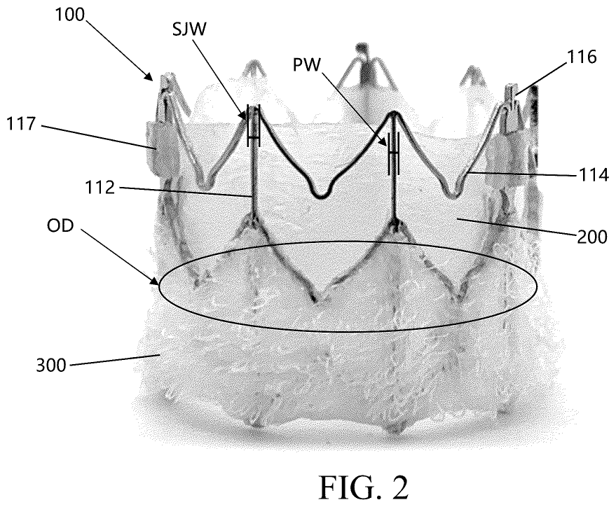

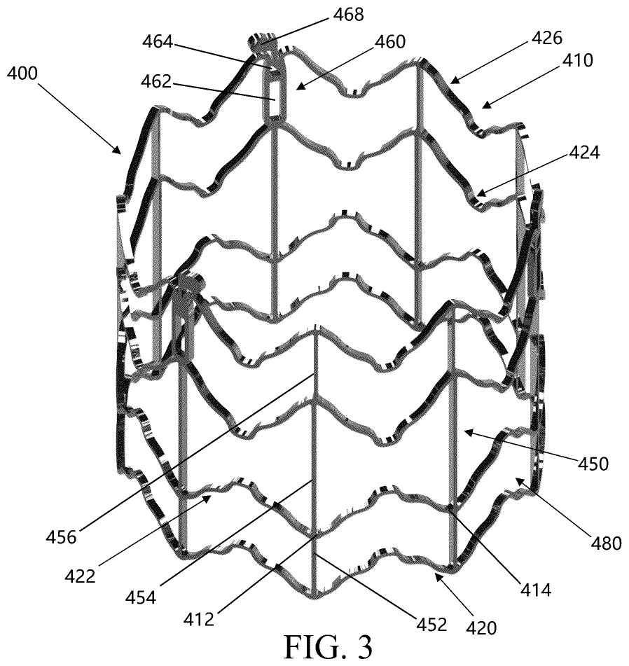

Referring now to FIGS. 1A-1E, these figures are illustrations of an implantable prosthetic heart valve 100 (e.g., TAV) and a method for inserting the prosthetic heart valve 100 in a valve region A (e.g., aortic valve, etc.) of a heart H. Prosthetic heart valve 100 can be implanted in the annulus of native aortic valve A; however, prosthetic heart valve 100 also can be configured to be implanted in other valves of the heart. Although the medical device illustrated is a TAV, the present disclosure is not limited to TAVs or any other heart valve replacement.

Referring now to FIG. 1A, prosthetic heart valve 100 generally comprises a frame 110 formed of a plurality of vertically extending vertically extending axial longitudinal members and angular articulating members 112, 114, strut joints 113, leaflet structure 200 supported by frame 110, and an outer skirt 300 secured to the outer surface of frame 110 and/or leaflet structure 200. Although not illustrated in FIG. 1A, the prosthetic heart valve 100 generally includes an inner skirt that is connected to the inner surface of the frame 110, and is also typically connected to leaflet structure 200 and/or the outer skirt 300. The frame can optionally include one or more an orientation structures or commissural markers 116. As illustrated in FIG. 1A, the one or more commissural markers 116 are generally is located on the top end of one or more of the vertically extending vertically extending axial longitudinal members 112. As illustrated in FIG. 1A, the strut joints 113 do not extend above the top ends of the vertically extending vertically extending axial longitudinal members 112 or below the bottom ends of the vertically extending vertically extending axial longitudinal members 112. As also illustrated in FIG. 1A, each of the commissural markers 116 extends above the top end of the vertically extending vertically extending axial longitudinal members 112.

Prosthetic heart valve 100 has a “lower” or proximal end 120 and an “upper” or distal end 130, wherein lower or proximal end 120 of prosthetic heart valve 100 is the inflow end and the upper or distal end 130 of prosthetic heart valve 100 is the outflow end.

Frame 110 can be optionally be coated with a polymer material (e.g., silicone, PTFE, ePTFE, polyurethane, polyolefins, hydrogels, biological materials [e.g., pericardium or biological polymers such as collagen, gelatin, or hyaluronic acid derivatives], etc.). The coating can be used to partially or fully encapsulate one or more of the vertically extending vertically extending axial longitudinal members 112 and/or non-vertically angular articulating members 114 on frame 110 and/or to partially or fully fill-in one or more of the openings between the angular articulating members 114 and/or vertically extending vertically extending axial longitudinal members 112.

Frame 110 can be optionally coated with a biological agent. As can be appreciated, other or additional components of the prosthetic heart valve 100 (e.g., outer skirt, inner skirt, leaflets, etc.) can optionally include the biological agent.

Frame 110 can be optionally coated with an enhancement coating (e.g., chromium nitride (CrN), diamond-like carbon (DLC), titanium nitride (TiN), titanium oxynitride or titanium nitride oxide (TiNOx), zirconium nitride (ZrN), zirconium oxide (ZrO₂), zirconium oxynitride (ZrNxOy) [e.g., cubic ZrN:O, cubic ZrO₂:N, tetragonal ZrO₂:N, and monoclinic ZrO₂:N phase coatings], oxyzirconium-nitrogen-carbon (ZrNC), zirconium OxyCarbide (ZrOC), etc.). As can be appreciated, other or additional components of the prosthetic heart valve 100 (e.g., outer skirt, inner skirt, leaflets, etc.) can optionally include the enhancement coating.

The outer skirt 300 can be formed of a variety of flexible materials (e.g., polymer (e.g., polyethylene terephthalate (PET), polyester, nylon, Kevlar, silicon, etc.), composite material, metal, fabric material, etc. In one non-limiting embodiment, the material used to partially or fully form outer skirt 300 can be substantially non-elastic (i.e., substantially non-stretchable and non-compressible). In another non-limiting embodiment, the material used to partially or fully form outer skirt 300 can be a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). Outer skirt 300 can optionally be formed from a combination of a cloth or fabric material that is coated with a flexible material or with a stretchable and/or compressible material so as to provide additional structural integrity to outer skirt 300. The size, configuration, and thickness of outer skirt 300 is non-limiting (e.g., thickness of 0.1-20 mils and all values and ranges therebetween). The outer skirt 300 can be secured to the frame 110, an optional inner skirt, and/or one or more leaflets of the leaflet structure 200 using various means (e.g., sutures, clips, clamp arrangement, stitching, etc.). At least a portion of the outer skirt 300 can optionally be located on the interior surface of frame 110; however, this is not required. In one non-limiting configuration, the outer skirt 300 is not located on the interior surface of frame 110.

Outer skirt 300 can be used to 1) at least partially seal and/or prevent perivalvular leakage, 2) at least partially secure leaflet structure 200 to frame 110, 3) at least partially protect one or more of the leaflets of leaflet structure 200 from damage during the crimping process of prosthetic heart valve 100, 4) at least partially protect one or more of the leaflets of leaflet structure 200 form damage during the operation of prosthetic heart valve 100 in heart H. In one non-limiting configuration, the outer skirt 300 is only connected to the inner skirt and frame 110, and wherein the outer skirt 300 is optionally only connected to the vertically extending vertically extending axial longitudinal members 112 of the frame 110, but not the angular articulating members 114 of the frame 110. In one non-limiting configuration, when the frame of the prosthetic heart valve is expanded in a treatment site, the perivalvular leak area about the expanded prosthetic heart valve is less than 20 mm² (0-20 mm² and all values and ranges therebetween). Prosthetic heart valve 100 can optionally include an inner skirt (not shown) that is positioned at least partially about the interior surface of the frame 110. The inner skirt (when used) generally is positioned completely around a portion of the inner surface of frame 110. Generally, the inner skirt is positioned about the inner surface of the lower portion of frame 110 and does not fully cover the upper portion of the inner surface of frame 110; however, this is not required. The inner skirt can be connected to frame 110, the outer skirt 300, and/or the leaflet structure 200 by a variety of arrangements (e.g., sutures, adhesive, melted connection, stitches, clamping arrangement, etc.). In one non-limiting configuration, the inner skirt is be connected to frame 110, the outer skirt 300, and the leaflets of the leaflet structure 200, and wherein the inner skirt is optionally only connected to the lower portion of the leaflets of the leaflet structure 200, and wherein the inner skirt is optionally connected to both the vertically extending vertically extending axial longitudinal members 112 of the frame 110 and the angular articulating members 114 of the frame 110, and wherein the inner skirt is optionally connected to only on row of angular articulating members 114 of the frame 110. In another non-limiting configuration, the bottom portion of the inner skirt is optionally connected to the bottom portion of the outer skirt 300, and wherein the bottom portion of the inner skirt and the bottom portion of the outer skirt 300 are optionally not connected to the frame 110, and the leaflets of the leaflet structure 200 are connected to the inner skirt at a location that is optionally above the connection location of the bottom portion of the inner skirt and the bottom portion of the outer skirt 300, and wherein a top portion of the inner skirt is connected to one row of angular articulating members 114 of the frame 110, and wherein the row of angular articulating members 114 of the frame 110 is that the inner skirt is connected thereto is optionally not the top row of angular articulating members 114 of the frame 110, and wherein a top portion of the outer skirt 300 is optionally connected to the inner skirt at a location that is below the angular articulating members 114 of the frame 110 that the inner skirt is connected thereto, and wherein the top profile of the inner skirt optionally has the same or similar shape as the profile of the angular articulating members 114 of the frame 110 to which the inner skirt is connected thereto, and wherein the top profile of the outer skirt 300 has the same or similar profile to the top profile of the inner skirt to which the outer skirt 300 is connected thereto. At least a portion of the inner skirt can optionally be located on the exterior surface of frame 110; however, this is not required.

Generally, the inner skirt is optionally formed of a less flexible and/or less compressible material than outer skirt 300; however, this is not required. The thickness of the inner skirt optionally has a thickness that is less than outer skirt 300; however, this is not required. The type of weave of the inner skirt can optionally be different from the outer skirt 300; however, this is not required.

The inner skirt can be formed of a variety of a stretchable and/or compressible material (e.g., silicone, PTFE, ePTFE, polyurethane, polyolefins, hydrogels, biological materials [e.g., pericardium or biological polymers such as collagen, gelatin, or hyaluronic acid derivatives], etc.). The inner skirt can optionally be formed from a combination of a cloth or fabric material that is coated with the stretchable and/or compressible material so as to provide additional structural integrity to the outer skirt. The size, configuration, and thickness of the outer skirt is non-limiting. The thickness of the inner skirt is generally 0.1-20 mils (and all values and ranges therebetween).

Leaflet structure 200 can be attached to frame 110 and/or inner skirt 300. The connection arrangement used to secure leaflet structure 200 to frame 110 and/or inner skirt 300 is non-limiting (e.g., sutures, melted bold, adhesive, stitching, clamp arrangement, etc.). The material used to form the one or more leaflets of leaflet structure 200 include, but are not limited to, polymers, bovine pericardial tissue, bovine tissue, porcine tissue, biocompatible synthetic materials, or various other suitable natural or synthetic materials.

Leaflet structure 200 can be comprised of two or more leaflets (e.g., 2, 3, 4, 5, 6, etc.). In one non-limiting arrangement, leaflet structure 200 includes three leaflets that are arranged to collapse in a tricuspid arrangement. The size, shape and configuration of the one or more leaflets of leaflet structure 200 are non-limiting. In one non-limiting arrangement, the leaflets generally have the same shape, size, configuration and thickness.

Two of more of the leaflets of leaflet structure 200 can optionally be secured to one another at their adjacent sides to form commissures 117 of leaflet structure 200 (the edges where the leaflets come together). Leaflet structure 200 can be secured to frame 110 and/or inner skirt 300 by a variety of connection arrangement (e.g., sutures, adhesive, melted bond, clamping arrangement, etc.). In one non-limiting configuration, the one or more leaflets of the leaflet structure 200 is connected to frame opening arrangement or commissural opening in the frame 110. In one non-limiting configuration, the commissures 117 of leaflet structure 200 is positioned at least partially through the frame opening arrangement or commissural opening and then secured to the frame 110 by one or more connection arrangements (e.g., sutures, adhesive, melted bond, clamping arrangement, etc.).

One or more leaflets of the leaflet structure 200 can optionally include reinforcing structures or strips to 1) facilitate in securing the leaflets together, 2) facilitate in securing the leaflets to the inner skirt, and/or frame 110, and/or 3) inhibit or prevent tearing or other types of damage to the leaflets.

Prosthetic heart valve 100 is configured to be radially collapsible to a collapsed or crimped state for introduction into the body on a delivery catheter (FIG. 1B) and radially expandable to an expanded state for implanting prosthetic heart valve 100 at a desired location in heart H (e.g., aortic valve A, etc.) (FIG. 1E).

The frame of prosthetic heart valve 100 is made of a plastically-expandable material that permits crimping of the frame to a smaller profile for delivery and expansion of prosthetic heart valve 100 using an expansion device. FIG. 1B illustrates a generic frame F of a prosthetic heart valve that is crimped on a generic balloon catheter C. The balloon B on the balloon catheter C can be used to expand the frame F from a crimped state to an expanded state. Various type of crimping apparatus and techniques can be used to crimp the prosthetic heart valve on the balloon delivery catheter. The process of crimping a prosthetic heart valve using a crimping device is known in the art and will not be described herein. During a crimping procedure, damage to leaflets of leaflet structure should be avoided.

As illustrated in FIGS. 1C-1E, once prosthetic heart valve 100 is crimped on balloon B of a catheter C, the distal portion of the catheter C is inserted through a blood vessel and to the location in heart H wherein prosthetic heart valve 100 is to be deployed (See FIG. 1C). At the treatment location, the balloon B on catheter C is expanded to thereby cause prosthetic heart valve 100 to be expanded and secured in a valve region A of heart H (See FIG. 1D). Thereafter, balloon B is deflated and the distal region of the catheter C is removed from the patient (See FIG. 1E).

Referring now to FIGS. 3-6M, several non-limiting embodiments of the frame 400 for prosthetic heart valve 100 is illustrated. Frame 400 configured to be crimped onto a delivery catheter C so that the crimped prosthetic heart valve 100 can be inserted in a heart valve. Frame 400 can optionally be configured to enable prosthetic heart valve 100 to be crimped to a diameter that is less than 22 Fr; however, this is not required. As such, prosthetic heart valve 100 that includes frame 400 in accordance with the present disclosure can optionally be configured to enable a prosthetic heart valve 100 to be inserted into smaller sized heart valves that could not previously be treated with prior art prosthetic heart valves. As can be appreciated, prosthetic heart valve 100 in accordance with the present disclosure can be sized and configured to be inserted in heart valves that are larger than 22 Fr.

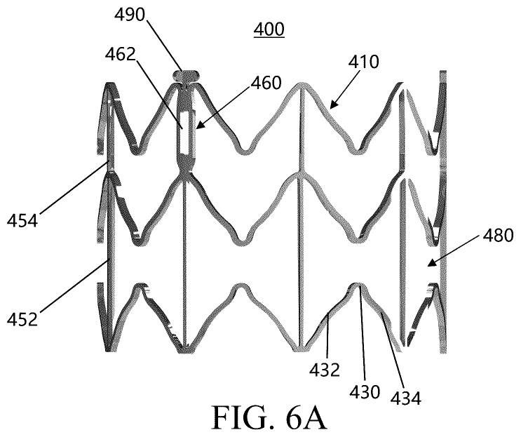

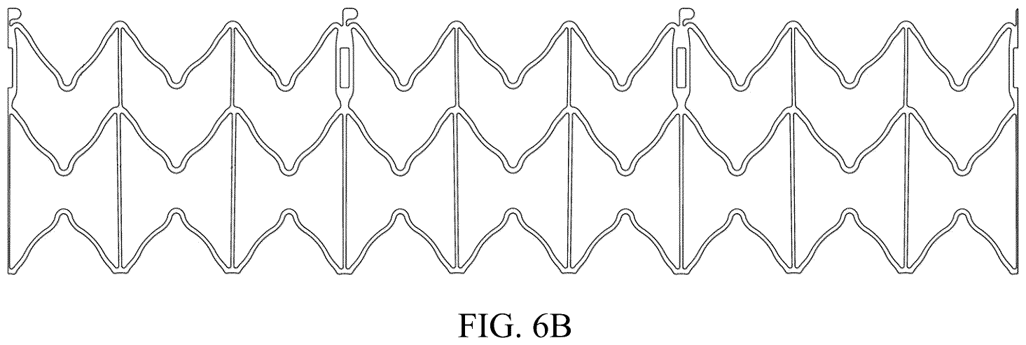

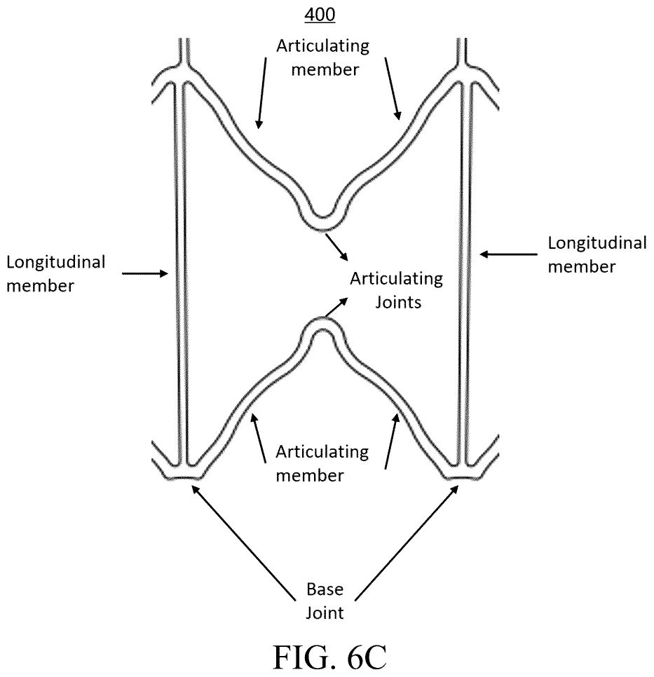

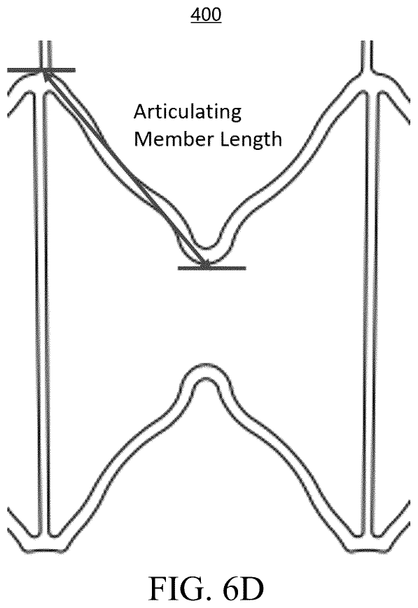

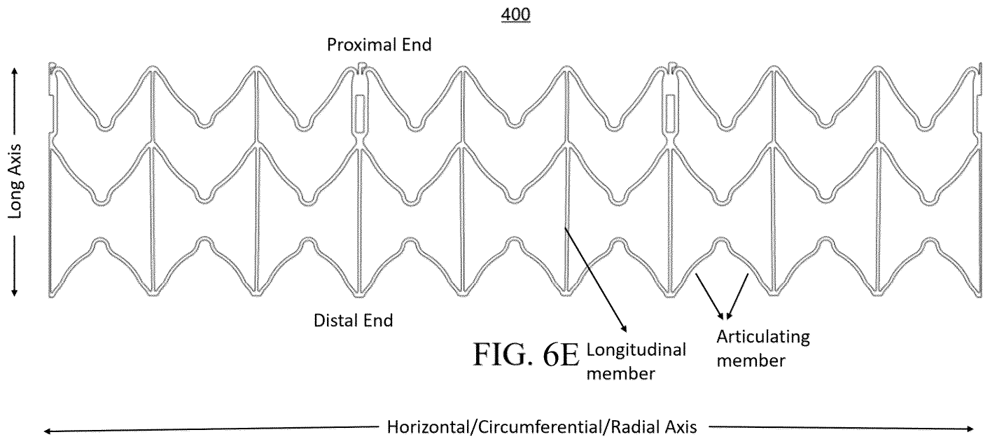

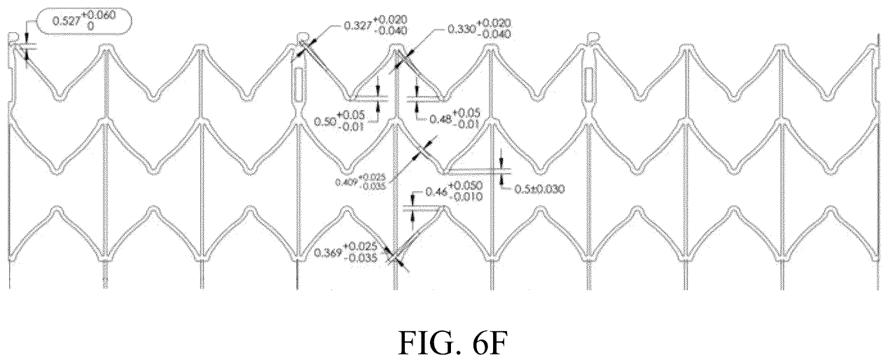

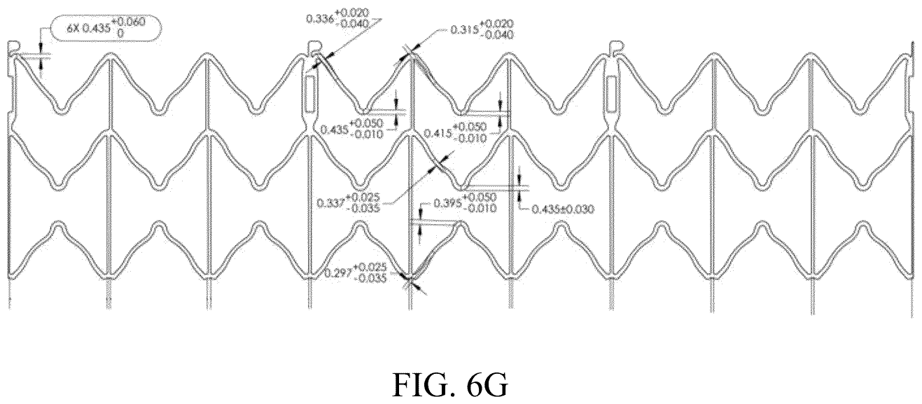

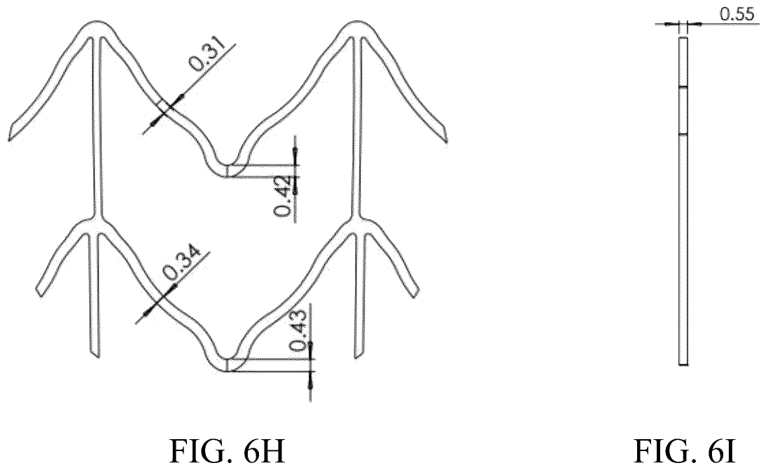

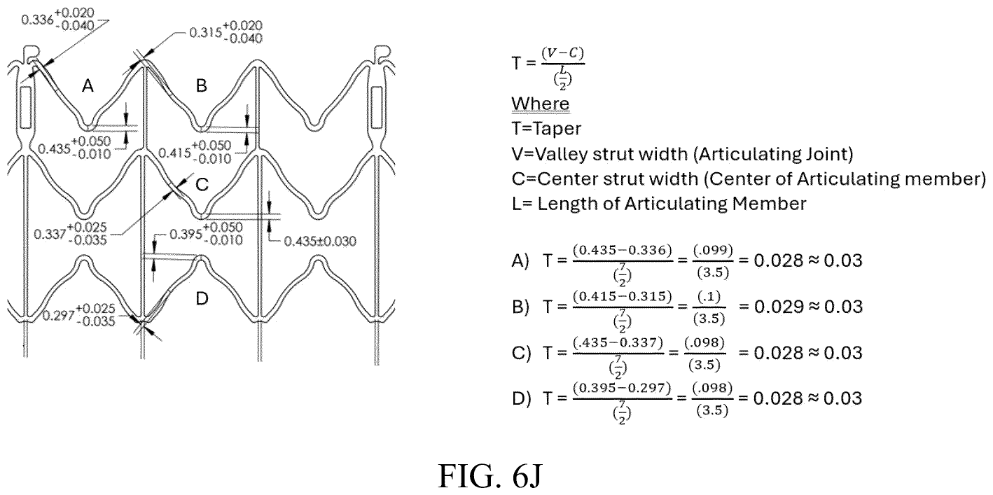

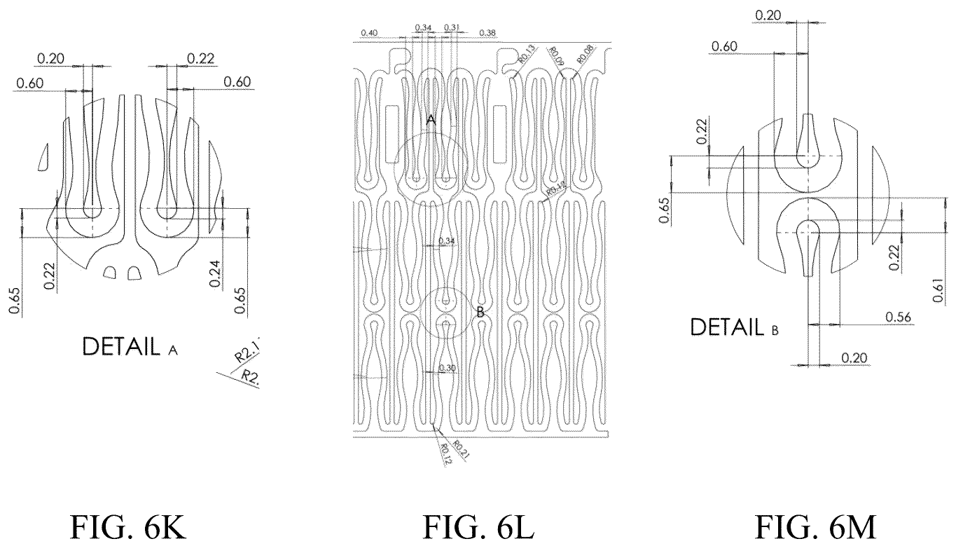

Referring now to FIGS. 3-5, one non-limiting embodiment of a frame 400 in accordance with the present disclosure is illustrated. FIGS. 6A-6M illustrates another non-limiting embodiment of a frame 400 in accordance with the present disclosure. As will be discussed in more detail below, frame 400 illustrated in FIGS. 3-5 includes four rows of angular articulating members 410 and three rows of cells wherein each row of cells included nine cells 480, and frame 400 illustrated in FIGS. 6A-6M includes three rows of angular articulating members 410 and two rolls of cells wherein each row of cells includes six cells 480. Each of the cells 480 illustrated in FIGS. 3-6M are open cells. As open cell is a cell 480 wherein at least one wall of the cell 480 is formed by an angular articulating member 410 and at least one wall of the cell 480 is formed by a vertically extending vertically extending vertically extending axial longitudinal member 450.

Referring again to FIGS. 3-5, the radially collapsible and expandable frame 400 includes plurality of angular articulating members 410, a plurality of vertically extending vertically extending vertically extending axial longitudinal member 450. A plurality of the vertically extending vertically extending vertically extending axial longitudinal member 450 include a frame opening arrangements 460 on a top region of the vertically extending vertically extending vertically extending axial longitudinal member 450. The plurality of angular articulating members 410 and the vertically extending vertically extending vertically extending axial longitudinal member 450 are connected together to form a plurality of cells 480 in frame 400. The frame opening arrangements 460 are also referred to as the commissural attachment area of the frame 400 wherein the leaflet arrangement 200 connects to the frame 400. Connected to the top region of the commissural attachment area can optionally include a top marker or orientation structure or commissural alignment marker 468.

The angular articulating members 410 have first and second ends 412, 414 that are connected to vertically extending vertically extending axial longitudinal members 450. The angular articulating members 410 that are located on the proximal end of the frame (i.e., inflow end) are oriented and configured on the frame such that 80-100% (and all values and ranges therebetween) of such angular articulating members 410 are positioned above the bottom end of the vertically extending vertically extending axial longitudinal members 450 when the frame is in the expanded and unexpanded configurations. The angular articulating members 410 that are located on the distal end of the frame (i.e., outflow end) are oriented and configured on the frame such that 80-100% (and all values and ranges therebetween) of such angular articulating members 410 are positioned below the top end of the vertically extending vertically extending axial longitudinal members 450 when the frame is in the expanded and unexpanded configurations.

Frame opening arrangements 460 that are located on the top portion of one or more of the vertically extending vertically extending vertically extending axial longitudinal member 450 can include a lower frame opening 462 and an optional an upper frame opening 464, 466. As can be appreciated, each of the frame opening arrangements 460 can include a single opening or more than two openings.

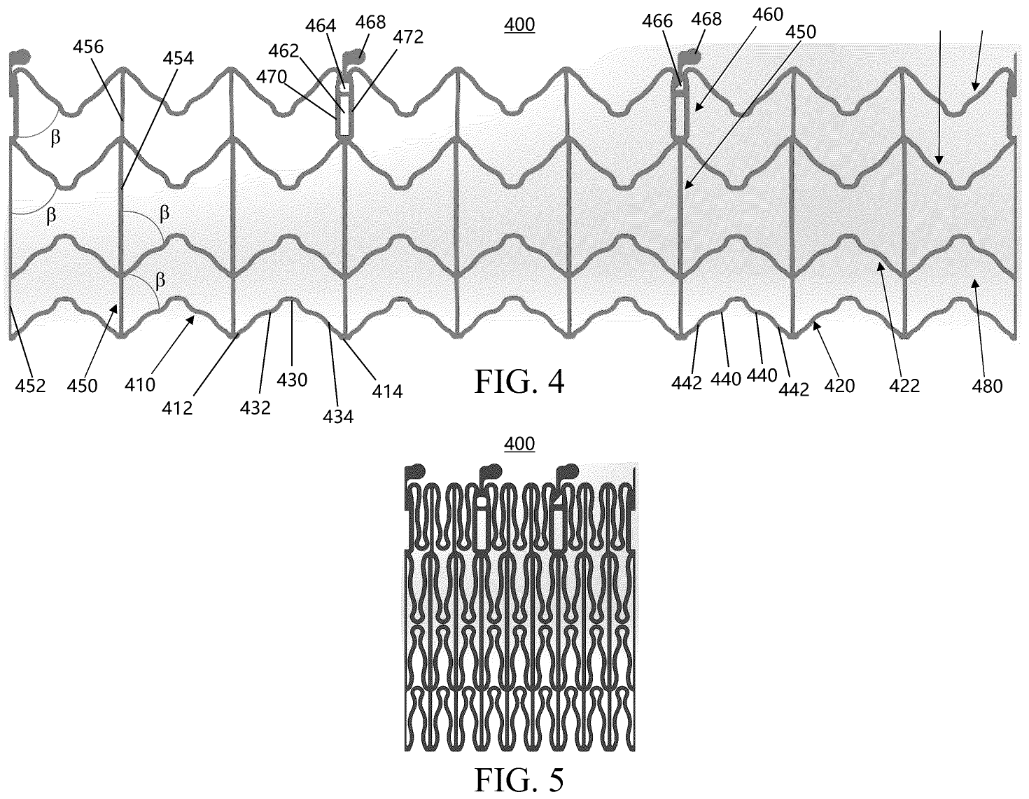

As illustrated in FIG. 4, frame 400 is formed of three rows of cells, wherein each row of cells includes nine cells 480. As illustrated in FIGS. 6A and 6B, frame includes two rows of cells, and wherein each row of cells includes nine cells. As illustrated in FIG. 4, the shape and size of cells 480 in each row cells have the same shape and size. As also illustrated in FIG. 4, the cells in each of the cells rows have a different shape, different size, and/or different orientation in frame 400 from the cells in adjacently positioned rows of cells.

Referring again to FIGS. 3-5, a plurality of vertically extending vertically extending vertically extending axial longitudinal member 450 are formed of a three vertically extending axial longitudinal member segments 452, 454, 456, and a plurality of the vertically extending vertically extending vertically extending axial longitudinal member 450 are formed of two vertically extending axial longitudinal member segment 452, 454 and frame opening arrangements 460. In one non-limiting embodiment, the frame 400 is formed from a single piece of material, and thus the three vertically extending axial longitudinal member segments 452, 454, 456 are formed of a single piece of material, and the vertically extending axial longitudinal member segment 452, 454 and frame opening arrangement 460 are also formed of a single piece of material. Likewise, the top marker or orientation structure or commissural alignment marker 468 and the frame opening arrangement 460 upon which the top marker or orientation structure or commissural alignment marker 468 is positioned are also formed of a single piece of material. As can be appreciated, one or more of the components of frame 400 can optionally be formed of a different piece of material that is connected to another component of the frame 400.

As illustrated in FIG. 3, each of the angular articulating members 410 have first and second articulating segments 432, 434 that are connected together by an articulating joint 430. The articulating joint 430 optionally has a C-shape or U-shape or curvilinear shape when the frame 400 is in both an expanded and unexpanded configuration. Referring now to FIGS. 6H-6M, in one non-limiting configuration, each of the angular articulating joints 430 optionally have an inner radius at least 0.12 mm (e.g., 0.12-0.3 mm and all values and ranges therebetween) when the frame 400 is a crimped orientation; and wherein a maximum width of each of the angular articulating joints 430 is optionally at least 0.2 mm (e.g., 0.2-0.8 mm and all values and ranges therebetween); and wherein a maximum cross-sectional area of each of the angular articulating joints 430 is optionally at least 0.03 mm². (e.g., 0.03-0.5 mm² and all values and ranges therebetween). One or more of the angular articulating members 410 can optionally be configured to have a variable cross-sectional area along a longitudinal length of the angular articulating member 410. In one non-limiting configuration, one or both articulating segments 432, 434 of the angular articulating members 410 has a first cross-sectional area at a proximal region of the articular segment, a second cross-sectional area at a central region of the articular segment, and a third cross-sectional area at a distal region at the articulating segment, and wherein a) the minimum second cross-sectional area at the central region is optionally less than a maximum first cross-sectional area at the proximal region, b) the minimum second cross-sectional area at the central region is optionally less than a maximum third cross-sectional area at the distal region, and/or c) the maximum first cross-sectional area at the proximal region is optionally less than a maximum third cross-sectional area at the distal region; and wherein the proximal end of the angular articulating member 410 connects to a vertically extending vertically extending vertically extending axial longitudinal member 450 and the distal end of the angular articulating member 410 connects to articulating joint 430. In another non-limiting configuration, a plurality of cells include one or both of the first and second articulating segments 432, 434 of the one or more of the angular articulating members 410 optionally have a taper T that is the same or is the same within 3% (e.g., 0-3% and all values and ranges therebetween) of one another based the taper formula of T=(V−C)/(L/2), wherein T is taper, V is the valley strut width (i.e., articulating joint 430 between two articulating segments 432, 434), C is the center strut width (i.e., width of the center of an articulating segment), and L is the length of an articulating segment. In one non-limiting configuration, the taper T is 0.01-0.06 (and all values and ranges therebetween).

Frame 400 illustrated in FIGS. 6A-6M includes a frame 400 that has a similar configuration as the frame illustrated in FIGS. 3-5 except that the frame 400 illustrated in FIGS. 6A-6M is formed of two rows of cell whereas FIGS. 3-5 illustrate a frame that has three rows of cells. The features of the frame illustrated in FIGS. 3-5 are equally applicable to frame 400 illustrated in FIGS. 6A-6M.

Referring against to FIGS. 3-6M, the vertically extending axial longitudinal member segments 452, 454, 456, or frame opening arrangement 460 that form each of vertically extending axial longitudinal members 450 are generally aligned along the longitudinal axis of vertically extending vertically extending axial longitudinal member 450. The thickness or cross-sectional area of each of vertically extending axial longitudinal members 450 along the longitudinal axis of the vertically extending axial longitudinal member can be constant or vary. The lower vertically extending axial longitudinal member segments 452 can a greater thickness or cross-sectional area than the upper vertically extending axial longitudinal member segments 456. The middle vertically extending axial longitudinal member segments 454 can have a greater thickness or cross-sectional area than upper vertically extending axial longitudinal member segments 456. The lower vertically extending axial longitudinal member segments 452 can have generally the same thickness or cross-sectional area as middle vertically extending axial longitudinal member segments 454. As can be appreciated, lower vertically extending axial longitudinal member segments 452 can have a different thickness or cross-sectional area as middle vertically extending axial longitudinal member segments 454. The cross-sectional shape of each the vertically extending axial longitudinal members 450 along the longitudinal length of vertically extending vertically extending axial longitudinal member 450 can be constant or vary. The longitudinal length of the vertically extending axial longitudinal member segments can be the same or different. The lower vertically extending axial longitudinal member segments 452 can have a longitudinal length that is less than a longitudinal length of either or both of middle vertically extending axial longitudinal member segments 454 and upper vertically extending axial longitudinal member segments 456, and the middle vertically extending axial longitudinal member segments 454 can have a longitudinal length that is greater than either or both lower vertically extending axial longitudinal member segments 452 and upper vertically extending axial longitudinal member segments 456. As illustrated in FIG. 4, lower vertically extending axial longitudinal member segments 452 has the shortest longitudinal length, and the middle vertically extending axial longitudinal member segments 454 has the longest longitudinal length.

As illustrated in FIGS. 3 and 4, frame 400 includes a first row 420 of angular articulating members 410, a second row 422 of angular articulating members 410, a third row 424 of angular articulating members 410, and a fourth row 426 of angular articulating members 410. First row 420 of angular articulating members 410 is the bottom row and fourth row 426 of angular articulating members 410 is the top row. The shape, size, and/or configuration of angular articulating members 410 of first row 420 are the same. The shape, size, and/or configuration of angular articulating members 410 on second row 422 are the same. The shape, size, and configuration of angular articulating members 410 of third row 424 are the same. The shape, size, and/or configuration of a plurality of angular articulating members 410 on fourth row 426 are the same and a plurality of angular articulating members 410 on fourth row 436 are different. Referring again to FIG. 4, angular articulating members 410 on fourth row 426, wherein either first end 412 or second end 414 the angular articulating members 410 is connected to frame opening arrangements 460, have a different shape, size, and/or configuration from angular articulating members 410 on fourth row 426 wherein both first end 412 and second end 414 of angular articulating members 410 are connected to vertically extending axial longitudinal members 450.

Referring again to FIGS. 3-6M, each of the angular articulating members 410 are formed of a centrally located arcuate portion or semi-circular portion or articulating joint 430, and first and second segments 432, 434 that extend from each side of semi-circular portion 430. First arm 432 terminates at first end 412 and second segment 434 terminates at second end 414. Each of first and second segment 432, 434 include one or more undulations 440, 442. As illustrated in FIG. 4, first articulating segment 432 includes first and second undulations 440, 442, wherein the first undulation 440 is located closer to semicircular portion or articulating joint 430 than the second undulation 442. Also, second articulating segment 434 includes first and second undulations 440, 442, wherein first undulation 440 is located closer to semicircular portion or articulating joint 430 than second undulation 442.

As best illustrated in FIG. 4, each of first and second articulating segments 432, 434 of all of angular articulating members 410 include two undulations; however, the shape and size of the undulations for two or more of the rows of angular articulating members 410 is different; however, this is not required. As also illustrated in FIG. 4, the shape and size of the undulations and the location of the undulations on angular articulating members 410 on each row of angular articulating members 410 are generally the same. As illustrated in FIG. 4, the shape and size of the undulations and the location of the undulations the angular articulating members 410 on first and second rows 420, 422 are the same or very similar (e.g., dimensions are less than 5% different). As also illustrated in FIG. 4, the shape and size of the undulations on angular articulating members 410 on the third row are different from first, second and fourth rows 420, 422, 426. Further, the shape and size of the undulations on angular articulating members 410 on the fourth row are different from first, second and third rows 420, 422, 424. In another non-limiting embodiment, for a plurality of angular articulating members 410, the length, shape and/or size of first and second articulating segments 432, 434 are the same or very similar (e.g., dimensions are less than 5% different). In one non-limiting configuration, angular articulating members 410 that form first row 420 of angular articulating members 410 have first and second articulating segments 432, 434 wherein the length, shape, and size of first and second articulating segments 432, 434 are the same. In another non-limiting configuration, angular articulating members 410 that form second row 422 of angular articulating members 410 have first and second articulating segments 432, 434 wherein the length, shape, and size the first and second articulating segments 432, 434 are the same. In another non-limiting configuration, the angular articulating members 410 that form third row 424 of angular articulating members 410 have first and second articulating segments 432, 434 wherein the length, shape, and size of first and second articulating segments 432, 434 are the same. In another non-limiting configuration, angular articulating members 410 that form fourth row 424 of angular articulating members 410 have first and second articulating segments 432, 434 wherein the length and shape of first and second articulating segments 432, 434 are not all the same. In another non-limiting configuration, angular articulating members 410 for first and second rows 420, 422 have first and second articulating segments 432, 434 wherein the length, shape, and size of first and second articulating segments 432, 434 are 410 of first and second articulating segments 432, 434 are the same or very similar (e.g., dimensions are less than 5% different) for angular articulating members 410 for first and second rows 420, 422. In another non-limiting configuration, angular articulating members 410 on each of first, second, third and fourth rows 420, 422, 424 and 426 a) have the same width, and/or b) the center point of semi-circular portion 430 is located with±5% (and all values and ranges therebetween) the midpoint between adjacently positioned vertically extending axial longitudinal members 450.

Referring again to FIGS. 3-6M, the spacing of angular articulating members 410 between adjacently positioned rows 420, 422, 424, 426 of angular articulating members 410 can be the same or different. In one non-limiting embodiment, the spacing of angular articulating members 410 between adjacent positioned rows (e.g., the first and second rows, the second and third rows, the third and fourth rows, etc.) is different. As illustrated in FIG. 4, the spacing between semi-circular portion 430 of first and second rows 420, 422 of angular articulating members 410 is greater than the spacing between semi-circular portion 430 of second and third rows 422, 424 of angular articulating members 410, and the spacing between first ends 412 of first and second rows 420, 422 of angular articulating members 410 is less than the spacing between first ends 412 of second and third rows 422, 424 of angular articulating members 410, and the spacing between second ends 414 of first and second rows 420, 422 of angular articulating members 410 is less than the spacing between second ends 414 of second and third rows 422, 424 of angular articulating members 410. As also illustrated in FIG. 4, semi-circular portion 430 of first and second rows 420, 422 of angular articulating members 410 are oriented toward the top of the frame, and semi-circular portion 430 of third and fourth rows 424, 425 of angular articulating members 410 are oriented toward the bottom of the frame. As such, the semi-circular portion 430 of second and third rows 422, 424 of angular articulating members 410 face one another. As also illustrated in FIG. 4, the spacing between semi-circular portion 430 of third and fourth rows 424, 426 of angular articulating members 410 is greater than the spacing between semi-circular portion 430 of first and second rows 420, 422 of angular articulating members 410, and the spacing between first ends 412 of third and fourth rows 424, 426 of angular articulating members 410 is greater than the spacing between first ends 412 of first and second rows 420, 422 of angular articulating members 410, and the spacing between second ends 414 of third and fourth rows 424, 426 of angular articulating members 410 is greater than the spacing between second ends 414 of first and second 420, 422 of angular articulating members 410. As also illustrated in FIG. 4, the spacing between semi-circular portion 430 of third and fourth rows 424, 426 of angular articulating members 410 is greater than the spacing between semi-circular portion 430 of second and third rows 422, 424 of angular articulating members 410, and the spacing between first ends 412 of third and fourth rows 424, 426 of angular articulating members 410 is less than the spacing between first ends 412 of second and third rows 422, 424 of angular articulating members 410, and the spacing between second ends 414 of third and fourth rows 424, 426 of angular articulating members 410 is less than the spacing between second ends 414 of second and third rows 422, 424 of angular articulating members 410.

As illustrated in FIGS. 6A-6M, the spacing of the angular articulating members in the adjacently positioned rows can be different.

Referring again to FIGS. 3-6M, frame opening arrangements 460 of the vertically extending vertically extending axial longitudinal member 450 are located between third and fourth rows 424, 426 of angular articulating members 410. As can be appreciated, one or more frame opening arrangements 460 can be located on other regions of frame 400. Frame opening arrangements 460 can optionally be used as securing locations for one of more leaflet structures 200; however, it can be appreciated that one or more of frame opening arrangements 460 can optionally be used as securing locations for other structures (e.g., leaflet, inner skirt, outer skirt, etc.), and/or be used as an indicator of the orientation and/or location of frame 400 in a body passageway or heart valve. Alternatively, an orientation structure 490 can be included in the frame 400. As illustrated in FIGS. 3-6M, each of frame opening arrangements 460 includes first and second frame opening struts 470, 472 that form a lower frame opening 462 and an optional an upper frame opening 464, 466 therebetween. The size and shape of lower frame opening 462 and optional an upper frame opening 464, 466 are non-limiting. As illustrated in FIGS. 3 and 4, lower frame opening 462 has a generally rectangular shape and extends only partially along the longitudinal length of frame opening arrangement 460. As can be appreciated, lower frame opening 462 can have other shapes and sizes. In one non-limiting configuration, each of frame opening arrangements 460 includes a lower frame opening 462 and lower frame openings 462 all have the same or very similar (e.g., dimensions are less than 5% different) shape and size. In one non-limiting embodiment, one or both of first and second frame opening struts 470, 472 a) has a longitudinal axis that is parallel to the longitudinal axis of vertically extending vertically extending axial longitudinal member 450, and/or b) has a longitudinal axis that is offset from the longitudinal axis of vertically extending vertically extending axial longitudinal member 450. As illustrated in FIGS. 3 and 4, both of first and second frame opening struts 470, 472 has a longitudinal axis that is parallel to the longitudinal axis of vertically extending vertically extending axial longitudinal member 450. The longitudinal length of one or both of first and second frame opening struts 470, 472 can be the same or less than the longitudinal length of length for a vertically extending axial longitudinal member segment that is located adjacent to first and second frame opening struts 470, 472. As illustrated in FIG. 4, the longitudinal length of first and second frame opening struts 470, 472 is about the same as the longitudinal length of length of vertically extending axial longitudinal member segment 456.

As illustrated in FIG. 4, the end of first or second articulating segments 432, 434 of angular articulating members 410 of fourth row 426 that is connected to frame opening arrangements 460 can optionally be configured to angle downwardly, and the other end of first or second articulating segments 432, 434 of angular articulating members 410 that is connected to a vertically extending axial longitudinal member segment is configured to angle upwardly. As illustrated in FIG. 4, the ends of first and second articulating segments 432, 434 of angular articulating members 410 of first, second and third rows 440, 422 and 424 that is connected to a vertically extending axial longitudinal member segment are both angled in the same direction. As illustrated in FIG. 4, the angle β of angular articulating members 410 relative to vertically extending axial longitudinal members 450 when the frame is in the expanded orientation is generally 25-60° (and all values and ranges therebetween). A similar arrangement regarding the connection of the angular articulating members to the vertically extending axial longitudinal member or frame opening arrangements is illustrated in FIGS. 6A-6M.

Referring now to FIGS. 3 and 4, frame opening arrangements 460 can optionally include one or more optional upper frame openings 464, 466. One or more optional upper frame openings 464, 466 are generally positioned above lower frame opening 462. Generally, one or more optional upper frame openings 464, 466 have a cross-sectional area or size that is less than lower frame opening 462; however, this is not required. As illustrated in FIGS. 3 and 4, the shape of two or more of optional upper frame openings 464, 466 are different. The different shapes of one or more optional upper frame openings 464, 466 can be used as a marker to facilitate in the proper positioning of frame 400 and prosthetic heart valve 100 in the heart. In one specific non-limiting configuration, each of one more optional upper frame openings 464, 466 has a different shape. As illustrated in FIG. 3, two of frame opening arrangements 460 include two different shaped upper frame openings 464, 466 and other frame opening arrangements 460 is absent an upper frame opening. As illustrated in FIG. 6, frame 400 is absent upper frame openings.

The top portion of each of frame opening arrangements 460 can optionally include a top marker 468. The shape and size of top marker 468 (when used) is non-limiting. As illustrated in FIGS. 3 and 4 and 6 the shape and size of markers 468 are the same or very similar (e.g., dimensions are less than 5% different). Top markers 468 can be used as a marker to facilitate in the proper positioning of frame 400 and prosthetic heart valve 100 in the heart. The one or more top markers 468 (when used) can also or alternatively be used to enable one or more components of prosthetic heart valve 100 (e.g., leaflet, inner skirt, outer skirt, etc.) to be connected to frame 400. The top markers 468 can be formed of the same or different material from other portions of frame 400.

Non-limiting dimensions of a frame 400 that is formed of the metal alloy in accordance with the present disclosure and which can be expanded to 26 mm can include a) vertically extending axial longitudinal members 450 having a length of 18-28 mm (and all values and ranges therebetween), b) a length of frame 400 in a flat state that is generally 70-95 mm (and all values and ranges therebetween), c) vertically extending axial longitudinal members 450 having a width that generally ranges between 0.2-0.7 mm (and all values and ranges therebetween), d) vertically extending axial longitudinal members 450 having a depth that is generally ranges between 0.2-0.7 mm (and all values and ranges therebetween), e) angular articulating members 410 having a width that generally ranges between 0.2-0.7 mm (and all values and ranges therebetween), f) angular articulating members 410 having a depth that generally ranges between 0.2-0.7 mm (and all values and ranges therebetween), g) a spacing of adjacently positioned vertically extending axial longitudinal members 450 that is generally 6-12 mm (and all values and ranges therebetween), h) the number of cells 480 in each of the sets of cells can be 2-20 (and all values and ranges therebetween). The width and/or depth of the lower vertically extending axial longitudinal members can optionally be greater than the one or more of the upper vertically extending axial longitudinal members. Likewise, the width and/or depth of the angular articulating members can optionally be greater than the one or more of the upper angular articulating members.

FIGS. 3 and 4 illustrate frame 400 in an expanded position and FIG. 5 illustrates frame 400 in the unexpanded or crimped position.

The frame 400 can be formed of a variety of materials. In one non-limiting configuration, frame 400 is partially or fully formed of a refractory metal alloy or a metal alloy that includes at least 15 awt. % rhenium or at least 15 awt. % hafnium.

Frame 110 of prosthetic heart valve 100, when formed of a refractory metal alloy or a metal alloy that includes at least 15 awt. % rhenium or at least 15 awt. % hafnium, can be crimped to have a crimped outer diameter that is a) at least 5% and up to a 33% smaller (e.g., 5-33% smaller and all value and ranges therebetween) than a crimped outer diameter of a frame of the same size, configuration, and shape that is formed of Co—Cr alloy; b) at least 5% and up to a 40% smaller (e.g., 5-40% smaller and all value and ranges therebetween) than a crimped outer diameter of a frame of the same size, configuration, and shape that is formed of Nitinol, and/or c) at least 5% and up to a 40% smaller (e.g., 5-40% smaller and all value and ranges therebetween) than a crimped outer diameter of a frame of the same size, configuration, and shape that is formed of TiAlV alloys.

A frame 400 for a prosthetic heart device (e.g., TAVR, etc.) that is formed of a refractory metal alloy or a metal alloy that includes at least 15 awt. % rhenium or at least 15 awt. % hafnium has one or more improved properties or advantages as compared to frames for prosthetic heart valves that are formed of Co—Cr alloy, TiAlV alloy, or NiTi alloy, namely 1) the outer diameter (OD) of the crimped prosthetic valve having a frame formed of a refractory metal alloy or a metal alloy that includes at least 15 awt. % rhenium or at least 15 awt. % hafnium is smaller than the OD crimped diameter of the crimped prosthetic valve having the same frame dimensions but formed of Co—Cr alloy, TiAlV alloy, or NiTi alloy, 2) the strut joint width on the frame (e.g., the location that the end of an angular articulating member and/or vertically extending axial longitudinal member is connected to another portion of the frame) that is formed a refractory metal alloy or a metal alloy that includes at least 15 awt. % rhenium or at least 15 awt. % hafnium can be less than the strut joint width on the frame formed of Co—Cr alloy, TiAIV alloy, or NiTi alloy while still forming a frame that is as strong as a frame formed by Co—Cr alloy, TiAlV alloy, or NiTi alloy, 3) the width of the angular articulating member and/or vertically extending axial longitudinal member on the frame that is formed a refractory metal alloy or a metal alloy that includes at least 15 awt. % rhenium or at least 15 awt. % hafnium can be less than the angular articulating member and/or vertically extending axial longitudinal member on the frame formed of Co—Cr alloy, TiAlV alloy, or NiTi alloy while still forming a frame that is as strong as a frame formed by Co—Cr alloy, TiAlV alloy, or NiTi alloy, 4) the amount of recoil of a frame that is formed of a refractory metal alloy or a metal alloy that includes at least 15 awt. % rhenium or at least 15 awt. % hafnium after the frame has been crimped or after the frame has been expanded is less than the amount of recoil of a frame having the same frame dimensions but formed of Co—Cr alloy, TiAlV alloy, or NiTi alloy, and/or 5) the amount of longitudinal foreshortening of a frame that is formed of a refractory metal alloy or a metal alloy that includes at least 15 awt. % rhenium or at least 15 awt. % hafnium after the frame has been expanded is less than the amount of longitudinal foreshortening of a frame having the same frame dimensions but formed of Co—Cr alloy, TiAlV alloy, or NiTi alloy.

The configuration of the frame as illustrated in FIGS. 3-6M results in little or no longitudinal foreshortening of a frame when the frame is expanded from a crimped state. Generally, the amount of longitudinal foreshortening of a frame from a crimped state to an expanded state is 0-20% (and all values and ranges therebetween), typically 0-15%, more typically 0-10%, and still more typically 0-5%. The orientation and configuration of the vertically extending axial longitudinal member segments (e.g., 452, 454, 456) and frame opening arrangements 460 facilitates in the reduction of longitudinal foreshortening of a frame when the frame is expanded from a crimped state. Likewise, the orientation and configuration of the vertically extending axial longitudinal member segments (e.g., 452, 454, 456) and frame opening arrangements 460 facilitates in the reduction of longitudinal foreshortening of a frame when the frame is crimped. A reduced amount of longitudinal foreshortening facilitates in ensuring the prosthetic heart implant when the frame is expanded from the crimped state maintains its proper position in the treatment area. Frames of prosthetic heart implant that foreshorten result in a reduction in longitudinal length when the frame is expanded. Such reduction in longitudinal length during expansion of the frame can result in the improper location of the expanded prosthetic heart implant in a treatment area, which improper location can result in a) improper operation of the implanted prosthetic heart implant, b) damage to the implanted prosthetic heart implant, c) potential damage to the tissue about the implanted prosthetic heart implant, d) reduced life of the prosthetic heart implant, and/or e) causing plaque and/or calcium deposits to form about the prosthetic heart implant.

The strength of a refractory metal alloy or a metal alloy that includes at least 15 awt. % rhenium or at least 15 awt. % hafnium used to partially or fully form frame 400 can optionally be greater than a cobalt-chromium alloy, nickel-titanium alloy, or a TiAlV alloy, thus the width of the angular articulating member and/or vertically extending axial longitudinal member and/or strut joints of frame 400 can be made smaller than frames formed of cobalt-chromium alloy, nickel-titanium alloy, or a TiAlV alloy, thereby enabling the frame to be made smaller without sacrificing the strength of the frame.

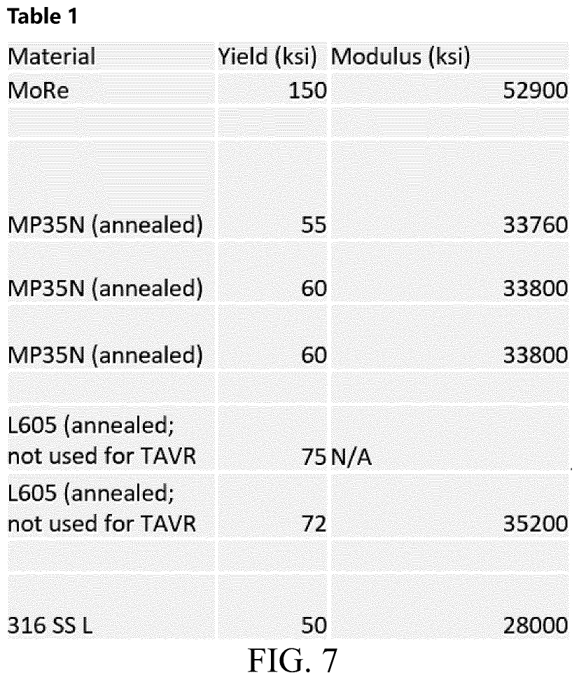

As illustrated in the Table 1 illustrated in FIG. 7, the Yield Strength and Young's Modulus (or Modulus of Elasticity) of a MoRe alloy (e.g., 45-55 wt. % Re & 45-55 wt. % Mo) is compared to two CoCr alloys (e.g., MP35N and L605) and a stainless-steel alloy (316L). As indicated in Table 1, the Yield Strength of the MoRe alloy is at least 2 times the Yield Strength of CoCr alloys such as MP35N and L605 and a stainless-steel alloy such as 316L. Also, the Young's Modulus of the MoRe alloy is at least 1.5 times the Young's Modulus of CoCr alloys such as MP35N and L605 and a stainless-steel alloy such as 316L. In one non-limiting embodiment, the metal alloy that is used to form 75%-100% (and all values and ranges therebetween) of the frame has a Yield Strength that is at least 1.1 times (e.g., 1.1-4 times and all values and ranges therebetween) of CoCr alloys, MP35N alloys, L605 alloys, SS alloys, stainless steel 316L alloy. In another non-limiting embodiment, the metal alloy (e.g., refractory metal alloy, refractory metal alloy that includes at least 25 wt. % rhenium, metal alloy that includes at least 15 awt. % rhenium) that is used to form 75%-100% (and all values and ranges therebetween) of the frame has a Young's Modulus that is at least 1.1 times (e.g., 1.1-2.5 times and all values and ranges therebetween) of CoCr alloys, MP35N alloys, L605 alloys, SS alloys, stainless steel 316L alloy.

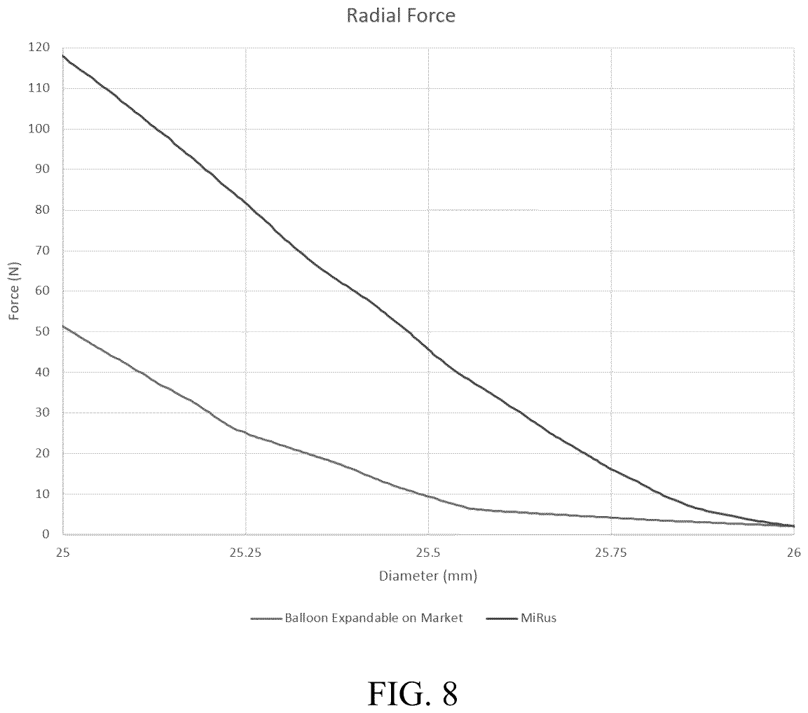

Referring now to FIG. 8, a graph provides a comparison of the radial force of two frames for a prosthetic heart valve that have the same size and configuration and wherein on of the frames is a MiRus™ frame that is formed of a MoRe alloy (e.g., 45-55 wt. % Re & 45-55 wt. % Mo) and the other frame is formed of a MP35N alloy. As illustrate din FIG. 8, for frames that are expanded up to 25.75 mm in diameter, the frame formed of a MoRe alloy has a larger radial strength that a frame formed of MP35N alloy. As such, the angular articulating member and/or vertically extending axial longitudinal member of the frame that is formed of MoRe alloy can be made thinner than a frame formed of MP35N alloy and still have the same or greater radial strength as a frame formed of MP35N alloy. Also, the greater radial force provided by the metal alloy allows for a larger open cell size in the frame as compared to prior art frames and smaller crimped profiles as compared to prior art frames.

The amount of recoil of a material used in the frame formed of a refractory metal alloy or a metal alloy that includes at least 15 awt. % rhenium or at least 15 awt. % hafnium when the frame is plastically deformed (e.g., crimped, expanded from the crimped state, etc.) can be less than the amount of recoil of a same sized and configured frame formed of cobalt-chromium alloy, nickel-titanium alloy, or a TiAlV alloy. The amount of recoil of a refractory metal alloy or a metal alloy that includes at least 15 awt. % rhenium or at least 15 awt. % hafnium when the frame is crimped or when the frame is expanded from a crimped stated is generally no more than 8% (e.g., 0-8% and all values and ranges therebetween), typically no more than 5%, more typically no more than 3%, still more typically no more than 2%, and even more typically less than 2%. Due to the low amount of recoil, the frame only needs to be subjected to a single crimping cycle to obtain the smallest crimping outer diameter of the crimped frame. Frames formed of metal alloys having a larger recoil typically need to be subjected to multiple crimping processes to obtain the designed side of the crimped frame.

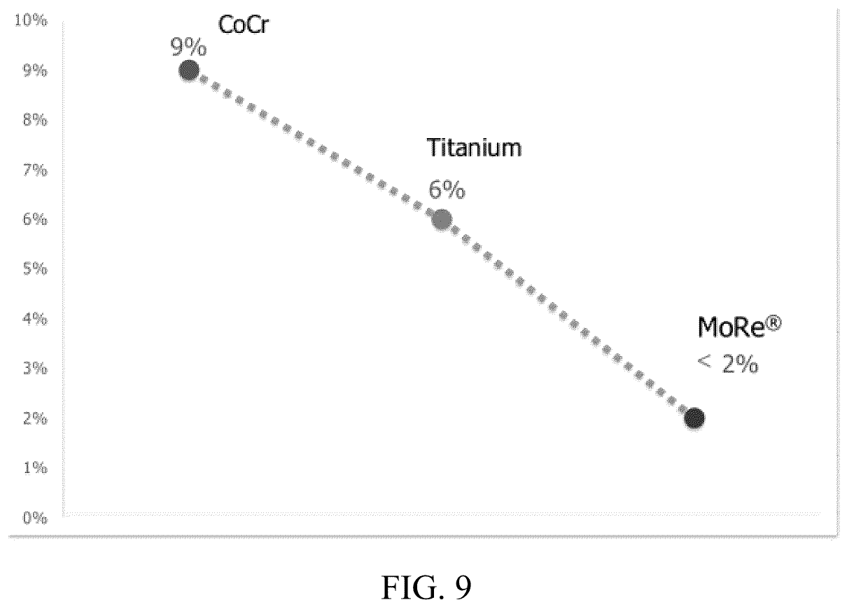

As illustrated in FIG. 9, the crimping of a frame that is formed of a) Co—Cr alloy (e.g., 35Co-35Ni-20Cr-10Mo) will recoil by 9% or more (e.g., 9-15% and all values and ranges therebetween) after the radial crimping forces are removed from the frame, or b) titanium alloy (e.g., e.g., Ti-6Al-4V) will recoil by 6% or more (e.g., 6-10% and all values and ranges therebetween) after the radial crimping forces are removed from the frame. FIG. 9 illustrates that a frame form of MoRe alloy (e.g., 45-55 wt. % Re & 45-55 wt. % Mo) will recoil less than 2% (e.g. 0.1-1.99% and all values and ranges therebetween) after being crimped or expanded. As such, when the frame is formed of a metal alloy that has reduced recoil, the need to subject the frame to multiple crimping cycles or procedures can eliminated, thereby a) reducing the incidence of damage to the frame, b) reducing the incidence of damage the leaflets of the prosthetic heart valve, c) reducing the incidence of damage the inner and/or outer skirt on the prosthetic heart valve, and/or d) reducing the incidence of damage to other components of the prosthetic heart valve (e.g., damage to balloon on the catheter, damage to one or more components on the catheter, etc.). The reduction in recoil after the expansion of the frame formed of a refractory metal alloy or a metal alloy that includes at least 15 awt. % rhenium or at least 15 awt. % hafnium results in the frame better conforming to the size of the orifice in the heart. As such, the increased EOA (effective orifice area) results in a reduction of perivalvular leak (i.e., a leak caused by a space between the patient's natural heart tissue and the valve replacement). The larger recoil of the frame formed of Co—Cr alloy or Ti alloy results in reduced EOA and increase amount of perivalvular leak about the prosthetic heart valve.

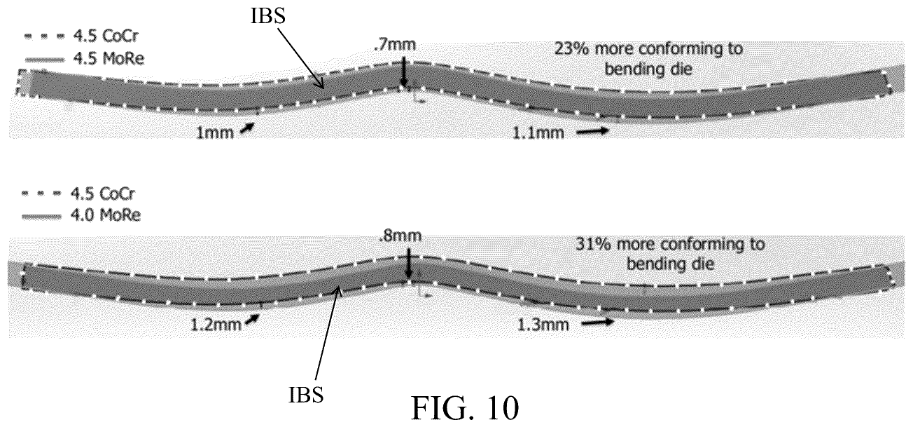

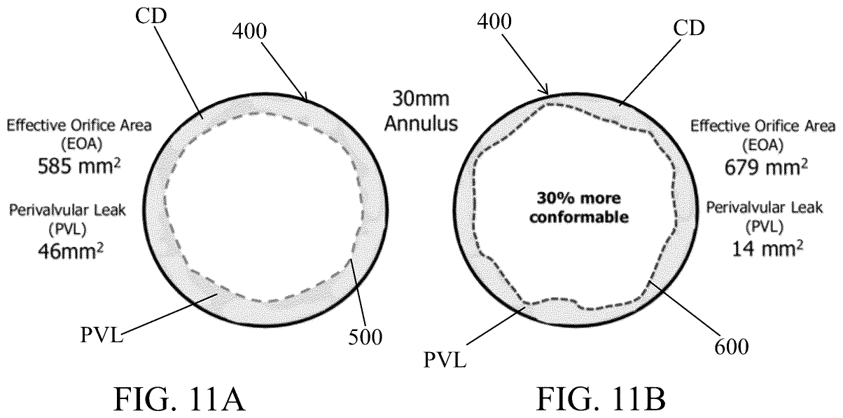

FIG. 10 illustrates two different wires formed of CoCr and a refractory metal such as MoRe to illustrate the conformability to bending of the two types of wires. When the frame of the prosthetic heart implant is expanded, the angular articulating member and/or vertically extending axial longitudinal member of the frame plastically deform (e.g., generally deform outwardly) due to the expansion of the inflatable balloon or from some other expansion device. Generally, the treatment location where the prosthetic heart implant is expanded is not perfectly cylindrical nor has a perfectly shaped circular cross-sectional shape. Generally, the treatment area is damaged and/or includes plaque, calcium deposits, and/or other materials (e.g., prior implanted medical devices, etc.) that cause the shape of the treatment area to be non-cylindrical-shaped or have a non-circular cross-sectional shape. As such, frames of prosthetic heart implants that can better conform to the irregular shapes in a treatment location result in a prosthetic heart implant that better fits the treatment area and can result in a reduction of perivalvular leak or other types of leakage about the outer perimeter of the expanded prosthetic heart implant. FIG. 10 illustrates that when same sized and configured angular articulating members and/or vertically extending axial longitudinal members of the frame that are formed of MoRe or Co—Cr alloy are subjected to the same bending force, the MoRe angular articulating member and/or vertically extending axial longitudinal member better conforms to the ideal bending shape IBS than the Co—Cr alloy angular articulating member and/or vertically extending axial longitudinal member. The two bending tests illustrate that the angular articulating member and/or vertically extending axial longitudinal member formed of refractory metal alloy such a MoRe had 23% and 31% better conformity to the ideal bending shape than the angular articulating member and/or vertically extending axial longitudinal member formed of CoCr. The ability to conform to a specific shape is largely dependent upon the recoil of the alloy used to form the angular articulating member and/or vertically extending axial longitudinal member. It has been found that angular articulating members and/or vertically extending axial longitudinal members formed of a refractory metal alloy or a metal alloy that includes at least 15 awt. % rhenium or at least 15 awt. % hafnium can have about 15-45% (and all values and ranges therebetween) better conformity to bending to an idea bending shape formed by a die than the same sized angular articulating members and/or vertically extending axial longitudinal members formed of Co—Cr alloy, TiAlV alloy, or Ni—Ti alloy. Such improved shape conformity results in improved conformity of an expanded prosthetic heart valve frame to a treatment area shape as illustrated in FIGS. 11A and 11B.

FIG. 11A illustrates the conformability of an expanded frame 500 formed of Co—Cr alloy in an irregularly shaped annulus 400 of a heart wherein the treatment area includes calcium deposits CD and leakage regions PVL about the outer perimeter of the expanded frame. The expanded frame forms an EOA of about 585 mm². Due to the inability of the CoCr alloy to readily conform to irregular shapes in the annulus, an open area of about 46 mm² is located about the outer perimeter of the expanded frame to allow for PVL about the expanded TAV. FIG. 11B illustrates the conformability of an expanded frame 600 formed of a refractory metal alloy such as MoRe in an irregularly shaped annulus 400 of a heart. The expanded frame forms an EOA of about 679 mm² due to the improved ability of a refractory metal alloy or a metal alloy that includes at least 15 awt. % rhenium or at least 15 awt. % hafnium to conform to irregular shapes in the annulus. Generally, the EOA of the expanded frame in accordance with the present disclosure is at least 600 mm². As such, only an area of about 14 mm² is located about the outer perimeter of the expanded frame. The expanded frame formed of refractory metal alloy is illustrated as being more than 30% (e.g., 30-40% and all values and ranges therebetween) more conformable to irregularly shaped annulus 400 of a heart as compared to the same shaped and sized frame Co—Cr alloy. In general, expandable frames formed of a refractory metal alloy or a metal alloy that includes at least 15 awt. % rhenium or at least 15 awt. % hafnium are about 10-50% (and all values and ranges therebetween) more conformable to irregularly shaped body passageways as compared to a same sized and configured frame formed of Co—Cr alloy, TiAIV alloy, or Ni—Ti alloy.

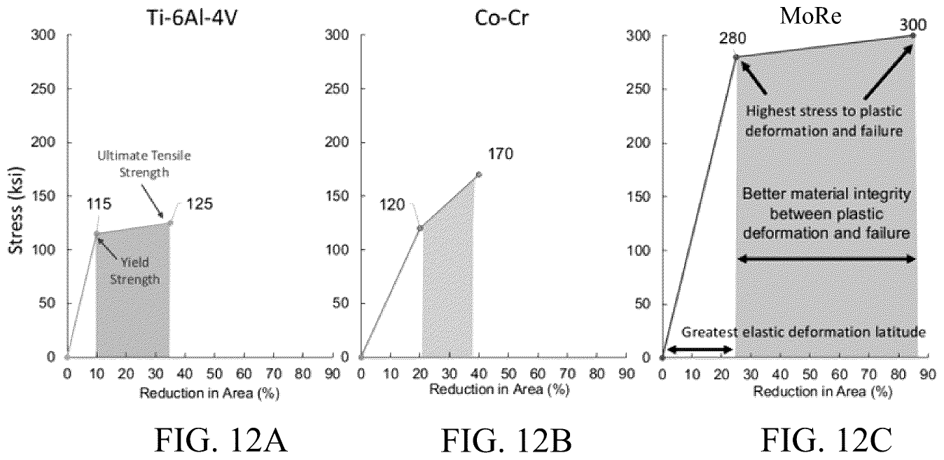

FIGS. 12A-12C illustrate stress vs. reduction in percent area graphs of angular articulating members and/or vertically extending axial longitudinal members formed of TiAlV alloy, CoCr alloy, and MoRe alloy. These graphs illustrate that angular articulating members and/or vertically extending axial longitudinal members in the frame formed of a refractory metal alloy or a metal alloy that includes at least 15 awt. % rhenium or at least 15 awt. % hafnium have improved properties such as strength, yield strength, ultimate tensile strength, fatigue ductility, greater deformation latitude, material integrity between plastic deformation and failure, and durability as compared to same sized and configured angular articulating members and/or vertically extending axial longitudinal members formed of CoCr alloy or TiAlV alloy. A refractory metal alloy or a metal alloy that includes at least 15 awt. % rhenium or at least 15 awt. % hafnium can have a strength of 1.5-5 times (and all values and ranges therebetween) greater than that of Co—Cr alloy, TiAIV alloy, or Ni—Ti alloy.

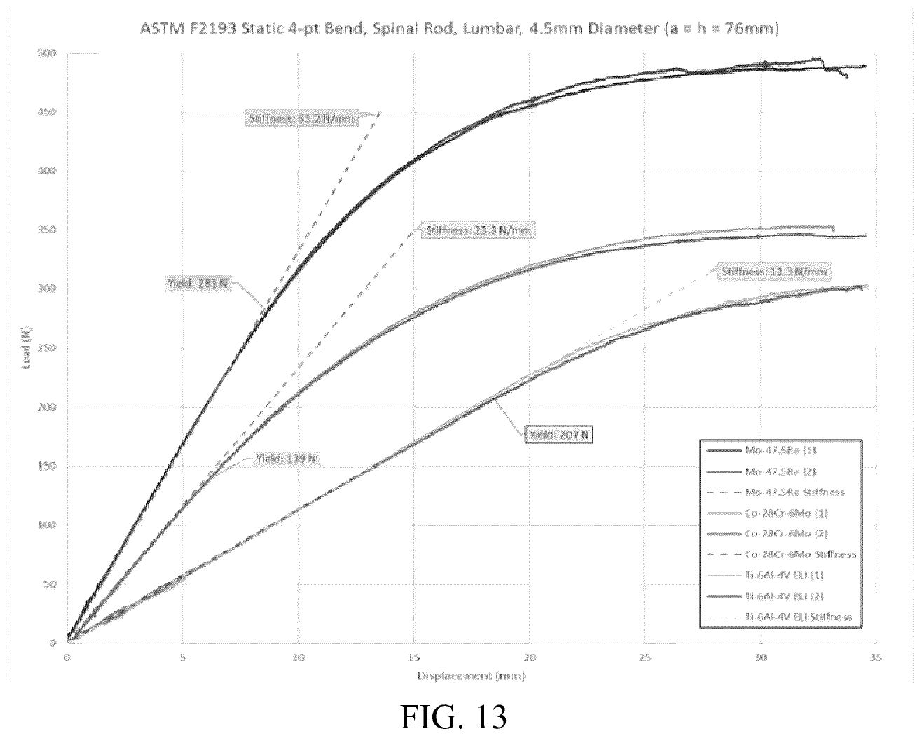

As illustrated in FIG. 13, an angular articulating member and/or vertically extending axial longitudinal member formed of a refractory metal alloy or a metal alloy that includes at least 15 awt. % rhenium or at least 15 awt. % hafnium has a greater stiffness and yield strength as compared to the same sized and configured angular articulating member and/or vertically extending axial longitudinal member formed of Co—Cr alloy, TiAIV alloy, or Ni—Ti alloy.

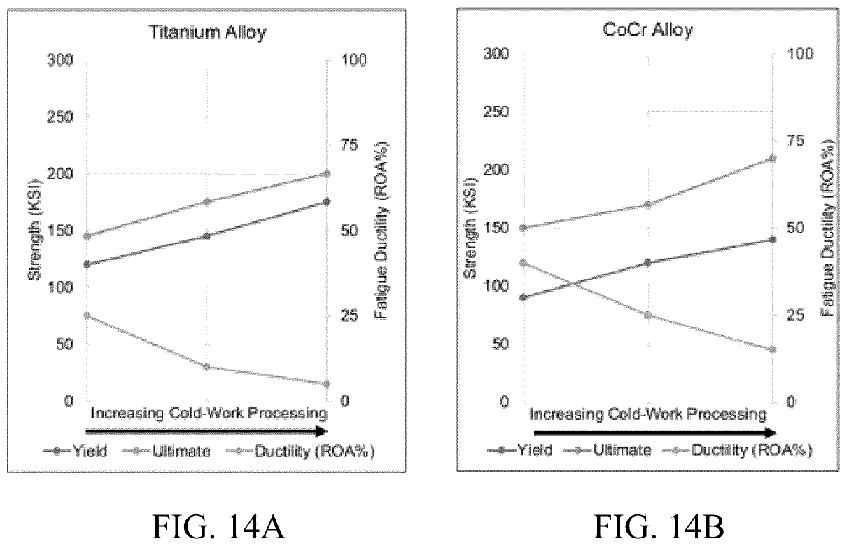

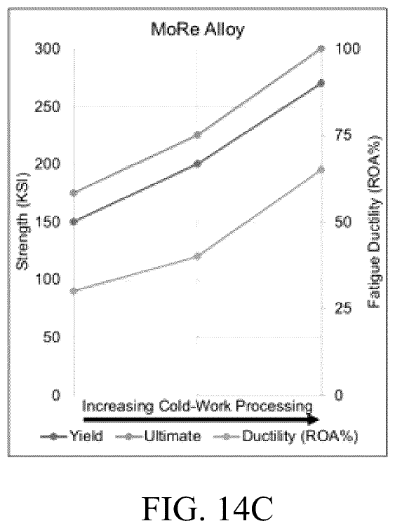

FIGS. 14A-14C are graphs that illustrate the yield strength, ultimate strength, and fatigue ductility of angular articulating members and/or vertically extending axial longitudinal members formed of TiAlV alloy, CoCr alloy, and MoRe alloy after such alloys are cold worked to reduce the cross-sectional area of the alloy. After being cold worked, a refractory metal alloy such as MoRe alloy has greater fatigue ductility, yield strength, and ultimate strength than Co—Cr alloys and TiAlV alloys. Also, the cold working of the MoRe alloy results in the increased ductility of the alloy, wherein CoCr alloys and TiAlV alloys have a reduction in ductility as additional cold working is applied to the alloy.

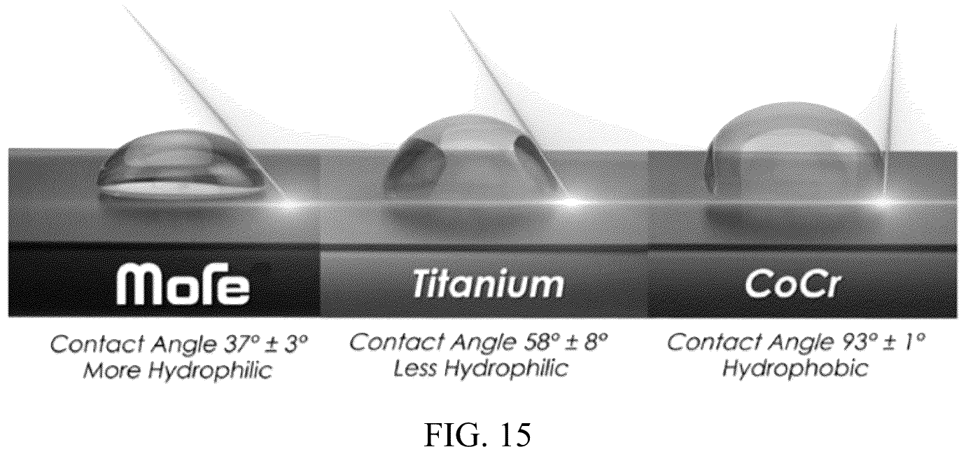

FIG. 15 illustrates the hydrophilicity of a refractory metal alloy such as a MoRe alloy compared to a Co—Cr alloy or TiAlV alloy. Hydrophilicity of a material implanted in a patient is an important property of the material with regard to the cell adhesion, cell migration, and cell multiplication of tissue on the material. As illustrated in FIG. 15, Co—Cr alloys are hydrophobic materials resulting in a large contact angle (93°±) 1° of a water droplet (e.g., distilled water) positioned on the surface of the Co—Cr alloy. TiAlV alloys are a little more hydrophilic than Co—Cr alloys and exhibit a contact angle of 58°±8° when a water droplet is positioned on the surface of the Ti alloy. Refractory metal alloys such as a MoRe alloy have a much greater hydrophilicity than Co—Cr alloys and TiAlV alloys. The MoRe alloy has a contact angle of 37°±3° when a water droplet is positioned on the surface of the MoRe alloy. A refractory metal alloy or a metal alloy that includes at least 15 awt. % rhenium or at least 15 awt. % hafnium generally have a hydrophilicity wherein the contact angle of a water droplet on the surface of the refractory metal alloy is 25°-45° (and all values and ranges therebetween), and typically 30-42°.

The reduced amount of recoil, improved bending conformity, and greater radial strength of expanded frames of prosthetic heart valves that are at least partially formed of a refractory metal alloy or a metal alloy that includes at least 15 awt. % rhenium or at least 15 awt. % hafnium as compared to same sized and configured expanded frames of prosthetic heart valves formed of Co—Cr alloy, TiAlV alloy, or Ni—Ti alloy results in the following non-limiting advantages: 1) formation of a frame for a prosthetic heart valve having thinner angular articulating members, vertically extending axial longitudinal members, and/or strut joints which results in i) safer vascular access when inserting the prosthetic heart valve through a body passageway and to the treatment area, and/or ii) decreased the risk of bleeding and/or damage to the body passageway and/or the treatment area when the prosthetic heart valve is delivered to the treatment area and/or expanded at the treatment area; 2) easier deliverability of the prosthetic heart valve to the treatment area which can result in i) decreased trauma to the body passageway (e.g., blood vessel, aortic arch trauma, etc.) during the insertion and/or expansion of the prosthetic heart valve at the treatment area, and/or ii) decreased risk of neuro complications-stroke; 3) less recoil which results in i) reduced crimping profile size, ii) increased conformability of the expanded prosthetic heart valve at the treatment area after expansion in the treatment area, iii) increased radial strength of the frame of the prosthetic heart valve after expansion at the treatment area, iv) only require a single crimping cycle to crimp the prosthetic heart valve on a balloon catheter or other type of delivery device, v) reduced incidence of damage to components of the prosthetic heart valve (e.g., angular articulating members, vertically extending axial longitudinal members, strut joints, and/or other components of the expandable frame, leaflets, skirts, coatings, etc.) during the crimping, expansion, and operation of the medical device, vi) greater effective orifice area (EOA) of the prosthetic heart valve after expansion of the medical device, vi) decreased pulmonary valve regurgitation (PVR) after expansion of the prosthetic heart valve in the treatment area, and/or vii) require only a single expansion cycle of the balloon on the balloon catheter or other expansion mechanism to fully expand the prosthetic heart valve; and/or 4) creating a prosthetic heart valve having superior material biologic properties to I) improve tissue adhesion and/or growth on or about prosthetic heart valve, II) reduce adverse tissue reactions with the prosthetic heart valve, III) reduced toxicity of prosthetic heart valve, IV) potentially decrease in-valve thrombosis during the life of the prosthetic heart valve, and/or V) reduce incidence of infection during the life of the prosthetic heart valve.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

What is claimed:

1. The expandable metal frame for a medical device includes one or more rows of cell wherein each cell is formed of vertically extending vertically extending axial longitudinal members and angular articulating members; said angular articulating members are formed of first and second articulating segments that are connected together by an angular articulating joint; a base joint that form a connection between said angular articulating member and said vertically extending axial longitudinal member; said each of the angular articulating joints have an inner radius at least 0.12 mm when the expandable frame is a crimped orientation; and wherein a maximum width of each of said angular articulating joints is at least 0.2 mm; and wherein a maximum cross-sectional area of each of said angular articulating joints is at least 0.03 mm².

2. The medical device as defined in claim 1, wherein said expandable metal frame is at least partially formed of an alloy that contains least 15 atw. % of rhenium.

3. The medical device as defined in claim 1, wherein said expandable metal frame is at least partially formed of an alloy that contains least 15 atw. % nickel and/or at least 15 awt. % titanium.

4. The medical device as defined in claim 1, wherein one or more of said angular articulating members has a taper defined as a change in the width along a longitudinal length of said angular articulating member; and wherein said taper is defined as a width of said angular articulating joint less the a width of a meeting point said angular articulating member to said base joint and then divided by a longitudinal length of said angular articulating joint divided by two; and wherein said taper is at least 0.015.

5. The medical device as defined in claim 1, wherein said expandable metal frame has longitudinal foreshortening of no more than 15%; and wherein said longitudinal foreshortening is a change in longitudinal length of said expandable metal frame from a collapsed orientation to an expanded orientation.

6. The medical device as defined in claim 1, wherein each of said cells of said frame is defined by an open area contained within one or more of said vertically extending axial longitudinal member and one or more angular articulating members; and wherein said expandable medical device has no more than 27 cells.

7. The medical device as defined in claim 1, where said medical device is a prosthetic valve that includes an inner skirt that is connected to said expandable metal frame, an outer skirt attached to said inner skirt, and one or more leaflets attached to said expandable metal frame.

8. The medical device as defined in claim 7, wherein said one or more leaflets are formed of porcine or bovine.

9. The medical device as defined in claim 8, wherein said leaflets are dry prior to use.

10. The medical device as defined in claim 1, wherein an effective orifice area of said expandable metal frame in an expanded orientation is greater than 600 mm².

11. The medical device as defined in claim 1, wherein said perivalvular leak of an expanded prosthetic heart valve in a treatment site is less than 20 mm².

12. The medical device as defined in claim 1, wherein a portion and all of said medical device includes an enhancement coating; and wherein said enhancement layer includes one or more of chromium nitride (CrN), diamond-like carbon (DLC), titanium nitride (TiN), titanium nitride oxide (TiNOx), zirconium nitride (ZrN), zirconium oxide (ZrO₂), zirconium-nitrogen-carbon (ZrNC), zirconium OxyCarbide (ZrOC), and zirconium oxynitride (ZrNxOy).

13. An expandable prosthetic heart valve comprising an expandable metal frame and at least one leaflet that is connected to said expandable metal frame; said expandable metal frame is configured to be crimped and further expand from a crimped orientation to an expanded orientation opening in a body passageway; said expandable metal frame has distal and proximal ends; said expandable metal frame includes a) an open cell configuration that includes a plurality of frame cells and at least two rows of frame cells, and/or b) is formed of a material that has a recoil of less than 10% thus resulting in reduced recoil of said expandable metal frame when expanded from said crimped orientation to said expanded orientation; said expandable metal frame has three or more of the following properties: i) at least 70-100% of said expandable metal frame is formed of a metal alloy that has a yield strength of at least 110 Ksi, ii) at least 70-100% of said expandable metal frame is formed of a metal alloy that has a modulus of elasticity of at least 35000 Ksi, iii) said expandable frame has a frame geometry that has a maximum of nine frame cells per horizontal row, iv) said expandable metal frame has longitudinal foreshortening along a longitudinal axis of said expandable frame of no more than 20% when said expandable metal frame is plastically deformed, v) each of said cells of said expandable frame includes at least one vertically extending axial longitudinal member and at least two angular articulating members, and wherein each of said angular articulating members includes a plurality of arcuate portions along a longitudinal length of said angular articulating member, vi) each of said cells of said expandable frame includes at least one vertically extending axial longitudinal member and at least two angular articulating members, and wherein each of said angular articulating members includes a plurality of arcuate portions along a longitudinal length of said angular articulating member, and wherein one or more of said vertically extending axial longitudinal members has a continuous linear shape of at least 80% of a longitudinal length of said vertically extending axial longitudinal member, and/or viii) the outer surface of said expandable frame includes an enhancement layer.

14. An expandable prosthetic heart valve comprising an expandable frame configured to expand from a crimped orientation to an expanded orientation when the prosthetic heart valve is positioned in a treatment site in a heart, wherein the expandable frame comprises:
a plurality of axial longitudinal members that are substantially linear and positioned radially about a longitudinal axis, wherein a longitudinal length of the expandable frame is not greater than a longitudinal length of the plurality of axial longitudinal members; and
a plurality of angular articulating members connected to the plurality of axial longitudinal members, wherein each angular articulating member is connected to a pair of adjacent axial longitudinal members.

15. The expandable prosthetic heart valve of claim 14, wherein when the expandable prosthetic heart valve is in a crimped orientation, the angular articulating members do not extend beyond the longitudinal members.

16. The expandable prosthetic heart valve of claim 14, wherein the expandable frame has no more than 20% foreshortening when expanded from a crimped orientation to an expanded orientation.

17. The expandable prosthetic heart valve of claim 14, wherein the plurality of axial longitudinal members and the plurality of angular articulating members form a plurality of cells.

18. The expandable prosthetic heart valve of claim 17, wherein the expandable frame includes two or more horizontal rows of cells.

19. The expandable prosthetic heart valve of claim 18, wherein at least one horizontal row of cells comprises no more than nine cells.

20. The expandable prosthetic heart valve of claim 14, wherein the angular articulating members of a first horizontal row and the angular articulating members of a second horizontal row extend toward either a top end or a bottom end of the expandable frame.

21. The expandable prosthetic heart valve of claim 20, wherein the angular articulating members of a first horizontal row and the angular articulating members of a second horizontal row extend toward one another.

22. The expandable prosthetic heart valve of claim 14, wherein the cells have an open cell geometry.

23. The expandable prosthetic heart valve of claim 14, wherein the expandable frame comprises a metal alloy that comprises at least 15 awt. % rhenium or at least 15 awt. % hafnium or a refractory metal alloy.

24. The expandable prosthetic heart valve of claim 14, wherein each angular articulating member comprises:
an intermediate arcuate portion, and
a pair of arms forming arcuate portions connected together at the intermediate arcuate portion, wherein each arm of the pair of arms is further connected to one axial longitudinal member at a connection point.

25. The expandable prosthetic heart valve of claim 24, wherein each of the angular articulating members in a first outer row and a second outer row extend longitudinally toward a center of the expandable frame.

26. An expandable prosthetic heart valve comprising an expandable frame configured to expand from a crimped orientation to an expanded orientation when the prosthetic heart valve is positioned in a treatment site in a heart, wherein the expandable frame comprises:
a plurality of axial longitudinal members that are substantially linear and positioned radially about a longitudinal axis; and
a plurality of angular articulating members connected to the plurality of axial longitudinal members, wherein each angular articulating member is connected to a pair of adjacent axial longitudinal members;
wherein when the expandable prosthetic heart valve is in a crimped orientation, the angular articulating members do not extend beyond the longitudinal members.
